SEQUENCE LISTING (152 sequences)

SEQ ID NO: 1 - protein, 6 aa, Artificial Sequence
  Synthetic construct
NIGTKN

SEQ ID NO: 2 - protein, 3 aa, Artificial Sequence
  Synthetic construct
DDS

SEQ ID NO: 3 - protein, 11 aa, Artificial Sequence
  Synthetic construct
QVWDSDNDRWL

SEQ ID NO: 4 - protein, 6 aa, Artificial Sequence
  Synthetic construct
GFTFSS

SEQ ID NO: 5 - protein, 8 aa, Artificial Sequence
  Synthetic construct
INTISTFI

SEQ ID NO: 6 - protein, 21 aa, Artificial Sequence
  Synthetic construct
ARERGNRFCDGDCYSGTTFDY

SEQ ID NO: 7 - protein, 114 aa, Artificial Sequence
  Synthetic construct
QPVLTQPPSVSVAPGRTSRITCEENNIGTKNVHWYQQKPGQAPLLVVHDDSDRPSGIPERFSGSNSGNTATLTISRVEAGDEADYYCQVWDSDNDRWLFGGGTRLTVLGQPKAA

SEQ ID NO: 8 - protein, 129 aa, Artificial Sequence
  Synthetic construct
QVQLVESGGGLVKPGGSLRLSCAASGFTFSSHSMNWVRQTPGKGLEWVSSINTISTFIYYADSVKGRFTVSRDNARNSLFLQMNTLTADDTGVYYCARERGNRFCDGDCYSGTTFDYWGPGTLVTVSSA

SEQ ID NO: 9 - protein, 6 aa, Artificial Sequence
  Synthetic construct
QDISNY

SEQ ID NO: 10 - protein, 3 aa, Artificial Sequence
  Synthetic construct
KAS

SEQ ID NO: 11 - protein, 10 aa, Artificial Sequence
  Synthetic construct
QQYEEYLRYS

SEQ ID NO: 12 - protein, 8 aa, Artificial Sequence
  Synthetic construct
GGTLSNYA

SEQ ID NO: 13 - protein, 8 aa, Artificial Sequence
  Synthetic construct
IVPMFSTT

SEQ ID NO: 14 - protein, 23 aa, Artificial Sequence
  Synthetic construct
ARDLPAPYCSADLCYKVLDAFDV

SEQ ID NO: 15 - protein, 108 aa, Artificial Sequence
  Synthetic construct
EIVLTQSPSSLSASVGDRVTITCQASQDISNYLNWYQQKPGKAPKLLIYKASSLESGVPSRFSGSGSGTEFTLTITRLQPDDFATYYCQQYEEYLRYSFGQGTKLEIK

SEQ ID NO: 16 - protein, 130 aa, Artificial Sequence
  Synthetic construct
QVQLVQSGAEVKKPGSSVKVSCRPPGGTLSNYAINWVRQAPGQGLEWMGGIVPMFSTTKYAQKFQGRLTLTADLSTITAHMELNRLTSEDTAVYYCARDLPAPYCSADLCYKVLDAFDVWGQGTLVTVSS

SEQ ID NO: 17 - protein, 6 aa, Artificial Sequence
  Synthetic construct
NIGRKH

SEQ ID NO: 18 - protein, 11 aa, Artificial Sequence
  Synthetic construct
QVWDNDAEQWV

SEQ ID NO: 19 - protein, 8 aa, Artificial Sequence
  Synthetic construct
GFTFSNFP

SEQ ID NO: 20 - protein, 8 aa, Artificial Sequence
  Synthetic construct
ISHDETMI

SEQ ID NO: 21 - protein, 20 aa, Artificial Sequence
  Synthetic construct
ARDTGVCSGGYCYTPRYFDQ

SEQ ID NO: 22 - protein, 107 aa, Artificial Sequence
  Synthetic construct
QPVLTQPPSVSVAPGKTADITCGGDNIGRKHVHWYRQRPSQAPLLVIYDDSDRPSGIPERLSGINSGNTATLTISGVEVGDEADYYCQVWDNDAEQWVFGGGTKLTV

SEQ ID NO: 23 - protein, 127 aa, Artificial Sequence
  Synthetic construct
EVQLVQSGGGAVQPGRSLRLSCAVSGFTFSNFPMHWVRQAPGRGLEWVAVISHDETMIYYLDSVKGRFTISRDNSRNIVYLQMHSLRVEDTAVYYCARDTGVCSGGYCYTPRYFDQWGQGTLVTVSS

SEQ ID NO: 24 - protein, 16 aa, Artificial Sequence
  Synthetic construct
ENFVPKEGVKKADKFI

SEQ ID NO: 25 - DNA, 19 bp, Artificial Sequence
  Synthetic construct
catcagcaaagacatgaca

SEQ ID NO: 26 - DNA, 1796 bp, Artificial Sequence
  Synthetic construct
accatggggggttctcatcatcatcatcatcatggtatggctagcatgactggtggacagcaaatgggtcgggatctgtacgacgatgacgataaggatcgatggggatccggcctgaacgatatttttgaagcgcagaaaattgaatggcatgaaggtggtggcggttccgaatcgaacaaacaaaacactgctagtacagaaaccacaacgacaaatgagcaaccaaaaccagaaagtagtgagctaactatcgaaaaagcaggtcagaaaatggatgatatgcttaactctaacgatatgattaagcttgctcccaaagaaatgccactagaatctgcagaaaaagaagaaaaaaagtcagaagacaaaaaaaagagcgaagaagatcacactgaagaaatcaatgacaagatttattcactaaattataatgagcttgaagtacttgctaaaaatggtgaaaccattgaaaattttgttcctaaagaaggcgttaagaaagctgataaatttattgtcattgaaagaaagaaaaaaaatatcaacactacaccagtcgatatttccattattgactctgtcactgataggacctatccagcagcccttcagctggctaataaaggttttaccgaaaacaaaccagacgcggtagtcaccaagcgaaacccacaaaaaatccatattgatttaccaggtatgggagacaaagcaacggttgaggtcaatgaccctacctatgccaatgtttcaacagctattgataatcttgttaaccaatggcatgataattattctggtggtaatacgcttcctgccagaacacaatatactgaatcaatggtatattctaagtcacagattgaagcagctctaaatgttaatagcaaaatcttagatggtactttaggcattgatttcaagtcgatttcaaaaggtgaaaagaaggtgatgattgcagcatacaagcaaattttttacaccgtatcagcaaaccttcctaataatcctgcggatgtgtttgataaatcagtgacctttaaagatttgcaacgaaaaggtgtcagcaatgaagctccgccactctttgtgagtaacgtagcctatggtcgaactgtttttgtcaaactagaaacaagttctaaaagtaatgatgttgaagcggcctttagtgcagctctaaaaggaacagatgttaaaacgaatggaaaatactctgatatcttagaaaatagctcatttacagctgtcgttttaggaggagatgctgcagagcacaataaggtggtcacaaaagactttgatgttattagaaacgttatcaaagacaatgctaccttcagtagaaaaaacccagcttatcctatttcatacaccagtgttttccttaaaaataataaaattgcgggtgtcaataacagaactgaatacgttgaaacaacatctaccgagtacactagtggaaaaattaacctgtctcatcaaggcgcgtatgttgctcaatatgaaatcctttgggatgaaatcaattatgatgacaaaggaaaagaagtgattacaaaacgacgttgggacaacaactggtatagtaagacatcaccatttagcacagttatcccactaggagctaattcacgaaatatccgtatcatggctagagaggccaccggcttagcttgggaatggtggcgaaaagtgatcgacgaaagagatgtgaaactatctaaagaaatcaatgtcaacatctcaggatcaaccttgagcccatatggttcgattacttataagtagga

SEQ ID NO: 27 - protein, 596 aa, Artificial Sequence
  Synthetic construct
MGGSHHHHHHGMASMTGGQQMGRDLYDDDDKDRWGSGLNDIFEAQKIEWHEGGGGSESNKQNTASTETTTTNEQPKPESSELTIEKAGQKMDDMLNSNDMIKLAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKDLQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMAREATGLAWEWWRKVIDERDVKLSKEINVNISGSGGSPYGSITYK

SEQ ID NO: 28 - DNA, 26 bp, Artificial Sequence
  Synthetic construct
caggtgcagctggtgcagtctggggc

SEQ ID NO: 29 - DNA, 26 bp, Artificial Sequence
  Synthetic construct
gccccagactgcaccagctgcacctg

SEQ ID NO: 30 - DNA, 26 bp, Artificial Sequence
  Synthetic construct
caggtccagctggtgcagtctggggc

SEQ ID NO: 31 - DNA, 26 bp, Artificial Sequence
  Synthetic construct
gccccagactgcaccagctggacctg

SEQ ID NO: 32 - DNA, 26 bp, Artificial Sequence
  Synthetic construct
caggtcaccttgaaggagtctggtcc

SEQ ID NO: 33 - DNA, 26 bp, Artificial Sequence
  Synthetic construct
ggaccagactccttcaaggtgacctg

SEQ ID NO: 34 - DNA, 26 bp, Artificial Sequence
  Synthetic construct
gaggtgcagctggtggagtctggggg

SEQ ID NO: 35 - DNA, 26 bp, Artificial Sequence
  Synthetic construct
cccccagactccaccagctgcacctc

SEQ ID NO: 36 - DNA, 26 bp, Artificial Sequence
  Synthetic construct
caggtgcagctgcaggagtcgggccc

SEQ ID NO: 37 - DNA, 26 bp, Artificial Sequence
  Synthetic construct
gggcccgactcctgcagctgcacctg

SEQ ID NO: 38 - DNA, 23 bp, Artificial Sequence
  Synthetic construct
gaggtgcagctggtgcagtctgg

SEQ ID NO: 39 - DNA, 23 bp, Artificial Sequence
  Synthetic construct
ccagactgcaccagctgcacctc

SEQ ID NO: 40 - DNA, 26 bp, Artificial Sequence
  Synthetic construct
caggtacagctgcagcagtcaggtcc

SEQ ID NO: 41 - DNA, 26 bp, Artificial Sequence
  Synthetic construct
ggacctgactgctgcagctgtacctg

SEQ ID NO: 42 - DNA, 23 bp, Artificial Sequence
  Synthetic construct
caggtgcagctggtgcaatctgg

SEQ ID NO: 43 - DNA, 23 bp, Artificial Sequence
  Synthetic construct
ccagattgcaccagctgcacctg

SEQ ID NO: 44 - DNA, 20 bp, Artificial Sequence
  Synthetic construct
gcagcccagggcsgctgtgc

SEQ ID NO: 45 - DNA, 20 bp, Artificial Sequence
  Synthetic construct
gcacagcsgccctgggctgc

SEQ ID NO: 46 - DNA, 22 bp, Artificial Sequence
  Synthetic construct
ghcatccrgwtgacccagtctc

SEQ ID NO: 47 - DNA, 22 bp, Artificial Sequence
  Synthetic construct
gagactgggtcawcyggatgdc

SEQ ID NO: 48 - DNA, 22 bp, Artificial Sequence
  Synthetic construct
gatrttgtgatgacycagwctc

SEQ ID NO: 49 - DNA, 22 bp, Artificial Sequence
  Synthetic construct
gagwctgrgtcatcacaayatc

SEQ ID NO: 50 - DNA, 22 bp, Artificial Sequence
  Synthetic construct
gaaatwgtrwtgacrcagtctc

SEQ ID NO: 51 - DNA, 22 bp, Artificial Sequence
  Synthetic construct
gagactgygtcawyacwatttc

SEQ ID NO: 52 - DNA, 23 bp, Artificial Sequence
  Synthetic construct
gacatcgtgatgacccagtctcc

SEQ ID NO: 53 - DNA, 23 bp, Artificial Sequence
  Synthetic construct
ggagactgggtcatcacgatgtc

SEQ ID NO: 54 - DNA, 22 bp, Artificial Sequence
  Synthetic construct
gaaacgacactcacgcagtctc

SEQ ID NO: 55 - DNA, 22 bp, Artificial Sequence
  Synthetic construct
gagactgcgtgagtgtcgtttc

SEQ ID NO: 56 - DNA, 22 bp, Artificial Sequence
  Synthetic construct
gawrttgtgmtgacwcagtctc

SEQ ID NO: 57 - DNA, 22 bp, Artificial Sequence
  Synthetic construct
gagactgwgtcakcacaaywtc

SEQ ID NO: 58 - DNA, 23 bp, Artificial Sequence
  Synthetic construct
gcacacaacagaggcagttccag

SEQ ID NO: 59 - DNA, 23 bp, Artificial Sequence
  Synthetic construct
ctggaactgcctctgttgtgtgc

SEQ ID NO: 60 - DNA, 26 bp, Artificial Sequence
  Synthetic construct
cagtctgtgytgackcagccrccctc

SEQ ID NO: 61 - DNA, 26 bp, Artificial Sequence
  Synthetic construct
gagggyggctgmgtcarcacagactg

SEQ ID NO: 62 - DNA, 21 bp, Artificial Sequence
  Synthetic construct
cagtctgccctgactcagcct

SEQ ID NO: 63 - DNA, 21 bp, Artificial Sequence
  Synthetic construct
aggctgagtcagggcagactg

SEQ ID NO: 64 - DNA, 26 bp, Artificial Sequence
  Synthetic construct
tcctatgagctgacwcagshvccckc

SEQ ID NO: 65 - DNA, 26 bp, Artificial Sequence
  Synthetic construct
gmgggbdsctgwgtcagctcatagga

SEQ ID NO: 66 - DNA, 26 bp, Artificial Sequence
  Synthetic construct
cagcctgtgctgactcartcvccctc

SEQ ID NO: 67 - DNA, 26 bp, Artificial Sequence
  Synthetic construct
gagggbgaytgagtcagcacaggctg

SEQ ID NO: 68 - DNA, 26 bp, Artificial Sequence
  Synthetic construct
cagcctgtgctgactcagccaacttc

SEQ ID NO: 69 - DNA, 26 bp, Artificial Sequence
  Synthetic construct
gaagttggctgagtcagcacaggctg

SEQ ID NO: 70 - DNA, 24 bp, Artificial Sequence
  Synthetic construct
aattttatgctgactcagccccac

SEQ ID NO: 71 - DNA, 24 bp, Artificial Sequence
  Synthetic construct
gtggggctgagtcagcataaaatt

SEQ ID NO: 72 - DNA, 24 bp, Artificial Sequence
  Synthetic construct
caggctgtggtgactcaggagccc

SEQ ID NO: 73 - DNA, 24 bp, Artificial Sequence
  Synthetic construct
gggctcctgagtcaccacagcctg

SEQ ID NO: 74 - DNA, 23 bp, Artificial Sequence
  Synthetic construct
cagactgtggtgacccaggagcc

SEQ ID NO: 75 - DNA, 23 bp, Artificial Sequence
  Synthetic construct
ggctcctgggtcaccacagtctg

SEQ ID NO: 76 - DNA, 23 bp, Artificial Sequence
  Synthetic construct
cagcctgtgctgactcagccacc

SEQ ID NO: 77 - DNA, 23 bp, Artificial Sequence
  Synthetic construct
ggtggctgagtcagcacaggctg

SEQ ID NO: 78 - DNA, 20 bp, Artificial Sequence
  Synthetic construct
cttgragctcctcagaggag

SEQ ID NO: 79 - DNA, 20 bp, Artificial Sequence
  Synthetic construct
ctcctctgaggagctycaag

SEQ ID NO: 80 - DNA, 38 bp, Artificial Sequence
  Synthetic construct
ccgccacaggcgcgcactcccaggtgcagctggtgcag

SEQ ID NO: 81 - DNA, 18 bp, Artificial Sequence
  Synthetic construct
ctgcaccagctgcacctg

SEQ ID NO: 82 - DNA, 42 bp, Artificial Sequence
  Synthetic construct
ccgccacaggcgcgcactcccagatcaccttgaaggagtctg

SEQ ID NO: 83 - DNA, 22 bp, Artificial Sequence
  Synthetic construct
cagactccttcaaggtgatctg

SEQ ID NO: 84 - DNA, 38 bp, Artificial Sequence
  Synthetic construct
ccgccacaggcgcgcactcccaggtgcagctggtggag

SEQ ID NO: 85 - DNA, 18 bp, Artificial Sequence
  Synthetic construct
ctccaccagctgcacctg

SEQ ID NO: 86 - DNA, 38 bp, Artificial Sequence
  Synthetic construct
ccgccacaggcgcgcactccgaggtgcagctggtggag

SEQ ID NO: 87 - DNA, 18 bp, Artificial Sequence
  Synthetic construct
ctccaccagctgcacctc

SEQ ID NO: 88 - DNA, 38 bp, Artificial Sequence
  Synthetic construct
ccgccacaggcgcgcactcccaggtgcagctgcaggag

SEQ ID NO: 89 - DNA, 18 bp, Artificial Sequence
  Synthetic construct
ctcctgcagctgcacctg

SEQ ID NO: 90 - DNA, 38 bp, Artificial Sequence
  Synthetic construct
ccgccacaggcgcgcactccgaggtgcagctggtgcag

SEQ ID NO: 91 - DNA, 18 bp, Artificial Sequence
  Synthetic construct
ctgcaccagctgcacctc

SEQ ID NO: 92 - DNA, 38 bp, Artificial Sequence
  Synthetic construct
ccgccacaggcgcgcactcccaggtacagctgcagcag

SEQ ID NO: 93 - DNA, 18 bp, Artificial Sequence
  Synthetic construct
ctgctgcagctgtacctg

SEQ ID NO: 94 - DNA, 40 bp, Artificial Sequence
  Synthetic construct
ccgccacaggcgcgcactcccaggtgcagctggtgcaatc

SEQ ID NO: 95 - DNA, 20 bp, Artificial Sequence
  Synthetic construct
gattgcaccagctgcacctg

SEQ ID NO: 96 - DNA, 60 bp, Artificial Sequence
  Synthetic construct
gggaagtagtccttgaccaggcagcccagggccgctgtgcccccagaggtgctcttggag

SEQ ID NO: 97 - DNA, 60 bp, Artificial Sequence
  Synthetic construct
ctccaagagcacctctgggggcacagcggccctgggctgcctggtcaaggactacttccc

SEQ ID NO: 98 - DNA, 38 bp, Artificial Sequence
  Synthetic construct
gggttccagctagccgcggtgacatccagatgacccag

SEQ ID NO: 99 - DNA, 18 bp, Artificial Sequence
  Synthetic construct
ctgggtcatctggatgtc

SEQ ID NO: 100 - DNA, 40 bp, Artificial Sequence
  Synthetic construct
gggttccagctagccgcggtgatattgtgatgactcagtc

SEQ ID NO: 101 - DNA, 20 bp, Artificial Sequence
  Synthetic construct
gactgagtcatcacaatatc

SEQ ID NO: 102 - DNA, 38 bp, Artificial Sequence
  Synthetic construct
gggttccagctagccgcggtgaaattgtgttgacacag

SEQ ID NO: 103 - DNA, 18 bp, Artificial Sequence
  Synthetic construct
ctgtgtcaacacaatttc

SEQ ID NO: 104 - DNA, 38 bp, Artificial Sequence
  Synthetic construct
gggttccagctagccgcggtgacatcgtgatgacccag

SEQ ID NO: 105 - DNA, 18 bp, Artificial Sequence
  Synthetic construct
ctgggtcatcacgatgtc

SEQ ID NO: 106 - DNA, 38 bp, Artificial Sequence
  Synthetic construct
gggttccagctagccgcggtgaaacgacactcacgcag

SEQ ID NO: 107 - DNA, 18 bp, Artificial Sequence
  Synthetic construct
ctgcgtgagtgtcgtttc

SEQ ID NO: 108 - DNA, 38 bp, Artificial Sequence
  Synthetic construct
gggttccagctagccgcggtgaaattgtgctgactcag

SEQ ID NO: 109 - DNA, 18 bp, Artificial Sequence
  Synthetic construct
ctgagtcagcacaatttc

SEQ ID NO: 110 - DNA, 59 bp, Artificial Sequence
  Synthetic construct
ctgggatagaagttattcagcaggcacacaacagaggcagttccagatttcaactgctc

SEQ ID NO: 111 - DNA, 36 bp, Artificial Sequence
  Synthetic construct
gagcagttgaaatctggaactgcctctgttgtgtgc

SEQ ID NO: 112 - DNA, 38 bp, Artificial Sequence
  Synthetic construct
atggatccggagtcgactctcagtctgtgytgackcag

SEQ ID NO: 113 - DNA, 18 bp, Artificial Sequence
  Synthetic construct
ctgmgtcarcacagactg

SEQ ID NO: 114 - DNA, 38 bp, Artificial Sequence
  Synthetic construct
atggatccggagtcgactctcagtctgccctgactcag

SEQ ID NO: 115 - DNA, 18 bp, Artificial Sequence
  Synthetic construct
ctgagtcagggcagactg

SEQ ID NO: 116 - DNA, 38 bp, Artificial Sequence
  Synthetic construct
atggatccggagtcgactcttcctatgagctgacwcag

SEQ ID NO: 117 - DNA, 18 bp, Artificial Sequence
  Synthetic construct
ctgwgtcagctcatagga

SEQ ID NO: 118 - DNA, 40 bp, Artificial Sequence
  Synthetic construct
atggatccggagtcgactctcagcctgtgctgactcartc

SEQ ID NO: 119 - DNA, 20 bp, Artificial Sequence
  Synthetic construct
gaytgagtcagcacaggctg

SEQ ID NO: 120 - DNA, 38 bp, Artificial Sequence
  Synthetic construct
atggatccggagtcgactctcagcctgtgctgactcag

SEQ ID NO: 121 - DNA, 18 bp, Artificial Sequence
  Synthetic construct
ctgagtcagcacaggctg

SEQ ID NO: 122 - DNA, 40 bp, Artificial Sequence
  Synthetic construct
atggatccggagtcgactctaattttatgctgactcagcc

SEQ ID NO: 123 - DNA, 20 bp, Artificial Sequence
  Synthetic construct
ggctgagtcagcataaaatt

SEQ ID NO: 124 - DNA, 38 bp, Artificial Sequence
  Synthetic construct
atggatccggagtcgactctcaggctgtggtgactcag

SEQ ID NO: 125 - DNA, 18 bp, Artificial Sequence
  Synthetic construct
ctgagtcaccacagcctg

SEQ ID NO: 126 - DNA, 38 bp, Artificial Sequence
  Synthetic construct
atggatccggagtcgactctcagactgtggtgacccag

SEQ ID NO: 127 - DNA, 18 bp, Artificial Sequence
  Synthetic construct
ctgggtcaccacagtctg

SEQ ID NO: 128 - DNA, 38 bp, Artificial Sequence; identical to SEQ ID NO: 120
  Synthetic construct

SEQ ID NO: 129 - DNA, 18 bp, Artificial Sequence
  Synthetic construct
ctgagtcagcacaggctg

SEQ ID NO: 130 - DNA, 59 bp, Artificial Sequence
  Synthetic construct
caccagtgtggccttgttggcttgaagctcctcagaggagggcgggaacagagtgaccg

SEQ ID NO: 131 - DNA, 39 bp, Artificial Sequence
  Synthetic construct
cggtcactctgttcccgccctcctctgaggagcttcaag

SEQ ID NO: 132 - DNA, 41 bp, Artificial Sequence
  Synthetic construct
ctgctgaataacttctatcccagagaggccaaagtacagtg

SEQ ID NO: 133 - DNA, 41 bp, Artificial Sequence
  Synthetic construct
cactgtactttggcctctctgggatagaagttattcagcag

SEQ ID NO: 134 - DNA, 40 bp, Artificial Sequence
  Synthetic construct
ggagtgcgcgcctgtggcggccgccaccaagaagaggatc

SEQ ID NO: 135 - DNA, 40 bp, Artificial Sequence
  Synthetic construct
gatcctcttcttggtggcggccgccacaggcgcgcactcc

SEQ ID NO: 136 - DNA, 40 bp, Artificial Sequence
  Synthetic construct
ctggtcaaggactacttccccgaaccggtgacggtgtcgt

SEQ ID NO: 137 - DNA, 40 bp, Artificial Sequence
  Synthetic construct
acgacaccgtcaccggttcggggaagtagtccttgaccag

SEQ ID NO: 138 - DNA, 40 bp, Artificial Sequence
  Synthetic construct
accgcggctagctggaacccagagcagcagaaacccaatg

SEQ ID NO: 139 - DNA, 40 bp, Artificial Sequence
  Synthetic construct
cattgggtttctgctgctctgggttccagctagccgcggt

SEQ ID NO: 140 - DNA, 40 bp, Artificial Sequence
  Synthetic construct
ccaacaaggccacactggtgtgtctcataagtgacttcta

SEQ ID NO: 141 - DNA, 40 bp, Artificial Sequence
  Synthetic construct
tagaagtcacttatgagacacaccagtgtggccttgttgg

SEQ ID NO: 142 - DNA, 40 bp, Artificial Sequence; identical to SEQ ID NO: 134
  Synthetic construct

SEQ ID NO: 143 - DNA, 40 bp, Artificial Sequence; identical to SEQ ID NO: 135
  Synthetic construct

SEQ ID NO: 144 - DNA, 40 bp, Artificial Sequence; identical to SEQ ID NO: 136
  Synthetic construct

SEQ ID NO: 145 - DNA, 40 bp, Artificial Sequence; identical to SEQ ID NO: 137
  Synthetic construct

SEQ ID NO: 146 - DNA, 40 bp, Artificial Sequence
  Synthetic construct
agagtcgactccggatccataagcaaggagtccgaggaga

SEQ ID NO: 147 - DNA, 40 bp, Artificial Sequence
  Synthetic construct
tctcctcggactccttgcttatggatccggagtcgactct

SEQ ID NO: 148 - DNA, 18 bp, Artificial Sequence
  Synthetic construct
catcatcatcatcatcat

SEQ ID NO: 149 - DNA, 1625 bp, Artificial Sequence
  Synthetic construct
gaatcgaacaaacaaaacactgctagtacagaaaccacaacgacaaatgagcaaccaaaaccagaaagtagtgagctaactatcgaaaaagcaggtcagaaaatggatgatatgcttaactctaacgatatgattaagcttgctcccaaagaaatgccactagaatctgcagaaaaagaagaaaaaaagtcagaagacaaaaaaaagagcgaagaagatcacactgaagaaatcaatgacaagatttattcactaaattataatgagcttgaagtacttgctaaaaatggtgaaaccattgaaaattttgttcctaaagaaggcgttaagaaagctgataaatttattgtcattgaaagaaagaaaaaaaatatcaacactacaccagtcgatatttccattattgactctgtcactgataggacctatccagcagcccttcagctggctaataaaggttttaccgaaaacaaaccagacgcggtagtcaccaagcgaaacccacaaaaaatccatattgatttaccaggtatgggagacaaagcaacggttgaggtcaatgaccctacctatgccaatgtttcaacagctattgataatcttgttaaccaatggcatgataattattctggtggtaatacgcttcctgccagaacacaatatactgaatcaatggtatattctaagtcacagattgaagcagctctaaatgttaatagcaaaatcttagatggtactttaggcattgatttcaagtcgatttcaaaaggtgaaaagaaggtgatgattgcagcatacaagcaaattttttacaccgtatcagcaaaccttcctaataatcctgcggatgtgtttgataaatcagtgacctttaaagatttgcaacgaaaaggtgtcagcaatgaagctccgccactctttgtgagtaacgtagcctatggtcgaactgtttttgtcaaactagaaacaagttctaaaagtaatgatgttgaagcggcctttagtgcagctctaaaaggaacagatgttaaaacgaatggaaaatactctgatatcttagaaaatagctcatttacagctgtcgttttaggaggagatgctgcagagcacaataaggtggtcacaaaagactttgatgttattagaaacgttatcaaagacaatgctaccttcagtagaaaaaacccagcttatcctatttcatacaccagtgttttccttaaaaataataaaattgcgggtgtcaataacagaactgaatacgttgaaacaacatctaccgagtacactagtggaaaaattaacctgtctcatcaaggcgcgtatgttgctcaatatgaaatcctttgggatgaaatcaattatgatgacaaaggaaaagaagtgattacaaaacgacgttgggacaacaactggtatagtaagacatcaccatttagcacagttatcccactaggagctaattcacgaaatatccgtatcatggctagagaggccaccggcttagcttgggaatggtggcgaaaagtgatcgacgaaagagatgtgaaactatctaaagaaatcaatgtcaacatctcaggatcaaccttgagcccatatggttcgattacttataagtagga

SEQ ID NO: 150 - DNA, 60 bp, Artificial Sequence
  Synthetic construct
ggcctgaacgatatttttgaagcgcagaaaattgaatggcatgaaggtggtggcggttcc

SEQ ID NO: 151 - DNA, 1625 bp, Artificial Sequence; identical to SEQ ID NO: 149
  Synthetic construct

SEQ ID NO: 152 - protein, 6 aa, Artificial Sequence
  Synthetic construct
HHHHHH

(19) United States
(12) Patent Application Publication
(10) Pub. No.: US 2024/0360204 A1
(43) Pub. Date: Oct. 31, 2024

(54) Human Monoclonal Antibodies Specific to Streptolysin O and Methods of Use

(71) Applicant: The United States Government as represented by the Department of Veterans Affairs, Washington, DC (US)

(72) Inventors: Sarah Hobdey, Boise, ID (US); Dennis L. Stevens, Boise, ID (US); Amy E. Bryant, Boise, ID (US); Emily Price, Boise, ID (US); Cheri L. Lamb-Mcfarlane, Boise, ID (US)

(21) Appl. No.: 18/563,412

(22) PCT Filed: May 27, 2022
(86) PCT No.: PCT/US22/31255

§ 371 Date: Nov. 22, 2023

Related U.S. Application Data
(60) Provisional application No. 63/193,878, filed on May 27, 2021.
(60) Provisional application No. 63/194,264, filed on May 28, 2021.

(51) Int. Cl. C07K 16/12; A61P 31/04

(52) U.S. Cl. CPC C07K 16/1275; A61P 31/04; C07K 2317/21; C07K 2317/76

(57) ABSTRACT

Disclosed herein are fully human anti-streptolysin O antibodies useful for treating necrotizing soft tissue infections in a subject. Also disclosed herein are methods of inhibiting or preventing streptolysin O-induced RBC-hemolysis and preventing or inhibiting cytotoxicity in epithelial cells by administering to a subject an effective amount of the anti-streptolysin O antibodies.

### CROSS REFERENCE TO RELATED APPLICATIONS

This application claims the benefit of U.S. Provisional Applications No. 63/193,878, filed May 27, 2021; and 63/194,264, filed May 28, 2021. The content of these earlier filed applications is hereby incorporated by reference herein in their entirety.

### INCORPORATION OF THE SEQUENCE LISTING

The present application contains a sequence listing that is submitted via EFS-Web concurrent with the filing of this application, containing the file name “37759_0361P1_SL.txt” which is 49,152 bytes in size, created on May 26, 2022, and is herein incorporated by reference in its entirety.

### BACKGROUND

Severe necrotizing soft tissue infections (NSTIs) can cause exceedingly high morbidity and mortality despite modern antibiotic regimens and intensive care measures. In each case, potent, well-characterized extracellular toxins mediate the rapid destruction of skin, subcutaneous tissues, fascia and muscle, often leading to multiple organ failure and toxic shock. Extensive tissue debridement, often including amputation, is important for source control. However, delays in diagnosis or surgical intervention allow circulating toxins that mediate tissue damage and organ failure to proceed unchecked. Thus, new therapeutic strategies are urgently needed for treating NSTIs.

### SUMMARY

Disclosed herein are isolated antibodies or antibody fragments thereof comprising: a) a light chain variable region, wherein the light chain variable region comprises a complementarity determining region light chain 1 (CDRL1) amino acid sequence of SEQ ID NO: 1, or SEQ ID NO: 1 having at least 1 conservative substitution; a determining region light chain 2 (CDRL2) amino acid sequence of SEQ ID NO: 2, or SEQ ID NO: 2 having at least 1 conservative substitution; and a determining region light chain 3 (CDRL3) amino acid sequence of SEQ ID NO: 3, or SEQ ID NO: 3 having at least 1 conservative substitution; and b) a heavy chain variable region, wherein the heavy chain variable region comprises a complementarity determining region heavy chain 1 (CDRH1) amino acid sequence of SEQ ID NO: 4, or SEQ ID NO: 4 having at least 1 conservative substitution; a complementarity determining region heavy chain 2 (CDRH2) amino acid sequence of SEQ ID NO: 5, or SEQ ID NO: 5 having at least 1 conservative substitution; and a complementarity determining region heavy chain 3 (CDRH3) amino acid sequence of SEQ ID NO: 6, or SEQ ID NO: 6 having at least 1 conservative substitution.

Disclosed herein are isolated antibodies or antibody fragments thereof comprising a light chain variable region amino acid sequence of SEQ ID NO: 7 and a heavy chain variable region amino acid sequence of SEQ ID NO: 8.

Disclosed herein are isolated antibodies or antibody fragments thereof comprising: a) a light chain variable region, wherein the light chain variable region comprises a complementarity determining region light chain 1 (CDRL1) amino acid sequence of SEQ ID NO: 9, or SEQ ID NO: 9 having at least 1 conservative substitution; a determining region light chain 2 (CDRL2) amino acid sequence of SEQ ID NO: 10, or SEQ ID NO: 10 having at least 1 conservative substitution; and a determining region light chain 3 (CDRL3) amino acid sequence of SEQ ID NO: 11, or SEQ ID NO: 11 having at least 1 conservative substitution; and b) a heavy chain variable region, wherein the heavy chain variable region comprises a complementarity determining region heavy chain 1 (CDRH1) amino acid sequence of SEQ ID NO: 12, or SEQ ID NO: 12 having at least 1 conservative substitution; a complementarity determining region heavy chain 2 (CDRH2) amino acid sequence of SEQ ID NO: 13, or SEQ ID NO: 13 having at least 1 conservative substitution; and a complementarity determining region heavy chain 3 (CDRH3) amino acid sequence of SEQ ID NO: 14, or SEQ ID NO: 14 having at least 1 conservative substitution.

Disclosed herein are isolated antibodies or antibody fragments thereof comprising a light chain variable region amino acid sequence of SEQ ID NO: 15 and a heavy chain variable region amino acid sequence of SEQ ID NO: 16.

Disclosed herein are isolated antibodies or antibody fragments thereof comprising: a) a light chain variable region, wherein the light chain variable region comprises a complementarity determining region light chain 1 (CDRL1) amino acid sequence of SEQ ID NO: 17, or SEQ ID NO: 17 having at least 1 conservative substitution; a determining region light chain 2 (CDRL2) amino acid sequence of SEQ ID NO: 2, or SEQ ID NO: 2 having at least 1 conservative substitution; and a determining region light chain 3 (CDRL3) amino acid sequence of SEQ ID NO: 18, or SEQ ID NO: 18 having at least 1 conservative substitution; and b) a heavy chain variable region, wherein the heavy chain variable region comprises a complementarity determining region heavy chain 1 (CDRH1) amino acid sequence of SEQ ID NO: 19, or SEQ ID NO: 19 having at least 1 conservative substitution; a complementarity determining region heavy chain 2 (CDRH2) amino acid sequence of SEQ ID NO: 20, or SEQ ID NO: 20 having at least 1 conservative substitution; and a complementarity determining region heavy chain 3 (CDRH3) amino acid sequence of SEQ ID NO: 21, or SEQ ID NO: 21 having at least 1 conservative substitution.

Disclosed herein are isolated antibodies or antibody fragments thereof comprising a light chain variable region amino acid sequence of SEQ ID NO: 22 and a heavy chain variable region amino acid sequence of SEQ ID NO: 23.

Disclosed herein are isolated antibodies or antibody fragments thereof comprising a light chain variable region amino acid sequence of SEQ ID NO: 7 and a heavy chain variable region amino acid sequence of SEQ ID NO: 8, wherein the isolated antibody comprises 1, 2, 3, 4, or 5 conservative amino acid substitutions in the light or heavy chain variable region amino acid sequences.

Disclosed herein are isolated antibodies or antibody fragments thereof comprising a light chain variable region amino acid sequence of SEQ ID NO: 14 and a heavy chain variable region amino acid sequence of SEQ ID NO: 15, wherein the isolated antibody comprises 1, 2, 3, 4, or 5 conservative amino acid substitutions in the light or heavy chain variable region amino acid sequences.

Disclosed herein are isolated antibodies or antibody fragments thereof comprising a light chain variable region amino acid sequence of SEQ ID NO: 22 and a heavy chain variable region amino acid sequence of SEQ ID NO: 23, wherein the isolated antibody comprises 1, 2, 3, 4, or 5 conservative amino acid substitutions in the light or heavy chain variable region amino acid sequences.

Disclosed herein are isolated antibodies or antibody fragments thereof comprising a light chain variable region and a heavy chain variable region, wherein the light chain variable region comprises: a) a variant complementarity determining region light chain 1 (CDRL1) comprising one or two conservative amino acid substitutions at positions 26-31 of SEQ ID NO: 7; b) a variant complementarity determining region light chain 2 (CDRL2) comprising one or two conservative amino acid substitutions at positions 49-51 of SEQ ID NO: 7; and c) a variant complementarity determining region light chain 3 (CDRL3) comprising one or two conservative amino acid substitutions at positions 88-98 of SEQ ID NO: 7; wherein the heavy chain variable region comprises: d) a variant complementarity determining region heavy chain 1 (CDRH1) comprising one or two conservative amino acid substitutions at positions 26-31 of SEQ ID NO: 8; e) a variant complementarity determining region heavy chain 2 (CDRH2) comprising one or two conservative amino acid substitutions at positions 51-58 of SEQ ID NO: 8; and f) a variant complementarity determining region heavy chain 3 (CDRH3) comprising one or two conservative amino acid substitutions at positions 97-117 of SEQ ID NO: 8.

Disclosed herein are isolated antibodies or antibody fragments thereof comprising a light chain variable region and a heavy chain variable region, wherein the light chain variable region comprises: a) a variant complementarity determining region light chain 1 (CDRL1) comprising one or two conservative amino acid substitutions at positions 27-32 of SEQ ID NO: 15; b) a variant complementarity determining region light chain 2 (CDRL2) comprising one or two conservative amino acid substitutions at positions 50-52 of SEQ ID NO: 15; and c) a variant complementarity determining region light chain 3 (CDRL3) comprising one or two conservative amino acid substitutions at positions 89-98 of SEQ ID NO: 15; wherein the heavy chain variable region comprises: d) a variant complementarity determining region heavy chain 1 (CDRH1) comprising one or two conservative amino acid substitutions at positions 26-33 of SEQ ID NO: 16; e) a variant complementarity determining region heavy chain 2 (CDRH2) comprising one or two conservative amino acid substitutions at positions 51-58 of SEQ ID NO: 16; and f) a variant complementarity determining region heavy chain 3 (CDRH3) comprising one or two conservative amino acid substitutions at positions 97-119 of SEQ ID NO: 16.

Disclosed herein are isolated antibodies or antibody fragments thereof comprising a light chain variable region and a heavy chain variable region, wherein the light chain variable region comprises: a) a variant complementarity determining region light chain 1 (CDRL1) comprising one or two conservative amino acid substitutions at positions 26-31 of SEQ ID NO: 22; b) a variant complementarity determining region light chain 2 (CDRL2) comprising one or two conservative amino acid substitutions at positions 49-51 of SEQ ID NO: 22; and c) a variant complementarity determining region light chain 3 (CDRL3) comprising one or two conservative amino acid substitutions at positions 88-98 of SEQ ID NO: 22; wherein the heavy chain variable region comprises: d) a variant complementarity determining region heavy chain 1 (CDRH1) comprising one or two conservative amino acid substitutions at positions 26-33 of SEQ ID NO: 23; e) a variant complementarity determining region heavy chain 2 (CDRH2) comprising one or two conservative amino acid substitutions at positions 51-58 of SEQ ID NO: 23; and f) a variant complementarity determining region heavy chain 3 (CDRH3) comprising one or two conservative amino acid substitutions at positions 97-119 of SEQ ID NO: 23.

### BRIEF DESCRIPTION OF THE DRAWINGS

FIGS. 1A-B show an antigen-specific B-cell isolation method. FIG. 1A shows class-switched B-cells separated from human PBMCs are the “pre-enriched” population. B-cells captured in the solid-phase matrix are “enriched” and those that flow-through are “depleted”. Mono-biotinylated SLOm-monomers are added to pre-enriched B-cells followed by streptavidin coated magnetic microbeads to capture the SLO-monomer-B-cell complex in the solid-phase matrix. FIG. 1B shows antigen specific B-cells from immunized donors are cultured (2-4 cells/well) for ˜12 days. As shown, on day 12 cells are collected by centrifugation and culture supernatants are evaluated for the presence of anti-SLOwt IgG by ELISA.

FIGS. 2A-B shows an antigen-specific B-cell cloning method. FIG. 2A shows variable heavy (VH) and light (VL) chains from B-cells from cultures in which supernatant were positive for anti-SLOwt IgG (FIG. 1) are amplified from cDNA by nested-PCR. VH and VL PCR products were sequenced (sequence shown is SEQ ID NO: 25) from which it can be determined if the transcript will generate a productive product. FIG. 2B shows productive PCR products cloned into the pVITRO vector.

FIG. 3 shows the specificity of the identified human monoclonal antibodies (huMAbs). In each panel: lane 1: molecular weight (MW) marker in kilodaltons; lane 2: biotinylated SLO; lane 3: non-biotinylated SLO; and lane 4: streptavidin. First panel on the left shows denaturing coomassie stained PAGE; and the five remaining panels to the right show western blot with huMAb primary as indicated and goat anti-human IgG-HRP for detection.

FIGS. 4A-D show the characterization of huMAb_{SLO}. Neutralization of SLO-mediated (FIG. 4A) red blood cell (RBC) lysis, (FIG. 4B) epithelial cell (A549) death, (FIG. 4C) RBC binding, and (FIG. 4D) A549 binding was plotted as a function of huMAb concentration in molarity (M). FIG. 4A shows that hemolysis was completed by standard 2% RBC lysis assay. Lysis was measured by supernatant absorbance at 550 nm and data were normalized between 0% and 100%. FIGS. 4B) and 4D) show that SLOwt-cell binding was determined by the ability of fluorescently SLOwt-tetramers to bind cell membranes after incubation with various concentrations of huMAb. Percent binding was determined by number of cells associated with SLO by flow cytometry. FIG. 4C) shows A549 cell death was determined by the percentage of cells stained with Zombie Green dye (fluorescence dead cell stain), by flow cytometry, after incubation with SLOwt and various concentrations on huMAb. In each graph, the 50% inhibition concentration for huMAb was determined by non-linear regression: Y=Bottom+(Top−Bottom)/(1+(X/IC50)), where top and bottom are the plateaus in the same units as Y. The results of these experiments are summarized in Table 7.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list. Further, the term “or” means “and/or” unless explicitly indicated to refer to alternatives only or the alternatives are mutually exclusive, although the disclosure supports a definition that refers to only alternatives and “and/or.”

As used herein, the term “another” means at least a second or more.

As used herein, the term “about” is used to indicate that a value includes the inherent variation of error for the device, the method being employed to determine the value, or the variation that exists among the study subjects.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. The term does not denote a particular age or sex. Thus, adult, child, adolescent and newborn subjects, as well as fetuses, whether male or female, are intended to be covered. As used herein, the term “subject” can refer to either a human or a non-human animal, such as primates, mammals, and vertebrates having a necrotizing soft tissue infection or diagnosed with a necrotizing soft tissue infection. In some aspects, the subject has had an exposure to pathogen associated with a necrotizing soft tissue infection. In some aspects, the subject in need will or is predicted to benefit from antibodies or antibody fragments disclosed herein treatment.

As used herein, the term “patient” refers to a subject afflicted with a disease or disorder. The term “patient” includes human and veterinary subjects. In some aspects of the disclosed methods, the “patient” has been diagnosed with a need for treatment for a necrotizing soft tissue infection, such as, for example, prior to the administering step.

As used herein, the term “treat,” “treatment,” or “treating” refers to administration or application of a therapeutic agent to a subject in need thereof, or performance of a procedure or modality on a subject, for the purpose of obtaining at least one positive therapeutic effect or benefit, such as treating an infection, disease or health-related condition. For example, a treatment can include administration of a pharmaceutically effective amount of an antibody, or a composition or formulation thereof that specifically binds to streptolysin O for the purpose of treating or preventing necrotizing soft tissue infections. The terms “treatment regimen,” “dosing regimen,” or “dosing protocol,” are used interchangeably and refer to the timing and dose of a therapeutic agent, such as an antibodies or antibody fragments disclosed herein.

As used herein, the term “therapeutic benefit” or “therapeutically effective” refers the promotion or enhancement of the well-being of a subject in need (e.g., a subject with or suspected of having a necrotizing soft tissue infection or diagnosed with a necrotizing soft tissue infection) with respect to the medical treatment, therapy, dosage administration, of a condition, particularly as a result of the use of the antibodies or antibody fragments disclosed herein described herein and the performance of the methods described herein. This includes, but is not limited to, a reduction in the frequency or severity of the signs or symptoms of an infection or disease. In some aspects, treatment of a necrotizing soft tissue infection may involve, for instance, the inhibiting or preventing streptolysin O-induced RBC-hemolysis, and preventing or inhibiting cytotoxicity in epithelial cells or preventing, inhibiting or reducing skin, muscle or other soft tissue death or destruction, pain, wound with fever, skin warmth or skin redness, skin blisters, pus-filled bumps, or numbness. Treatment of necrotizing soft tissue infections may also refer to achieving a sustained response in a subject or prolonging the reducing the risk of sepsis, shock, organ failure or death.

As used herein, the term “administer” or “administration” refers to the act of physically delivering, e.g., via injection or an oral route, a substance as it exists outside the body into a patient, such as by oral, subcutaneous, mucosal, intradermal, intravenous, intramuscular delivery and/or any other method of physical delivery described herein or known in the art. When a disease, disorder or condition, or a symptom thereof, is being treated therapeutically, administration of the substance typically occurs after the onset of the disease, disorder or condition or symptoms thereof. Prophylactic treatment involves the administration of the substance at a time prior to the onset of the disease, disorder or condition or symptoms thereof.

As used herein, the term “effective amount” refers to the quantity or amount of a therapeutic (e.g., an antibody or pharmaceutical composition provided herein) which is sufficient to reduce, diminish, alleviate, and/or ameliorate the severity and/or duration of a necrotizing soft tissue infection or a symptom related thereto. This term also encompasses an amount necessary for the reduction or amelioration of the advancement or progression of a necrotizing soft tissue infection; the reduction or amelioration of the recurrence, development of a necrotizing soft tissue infection or onset of necrotizing soft tissue infection; and/or the improvement or enhancement of the prophylactic or therapeutic effect(s) of another therapy. In some aspects, the effective amount of an antibody provided herein is from about or equal to 0.1 mg/kg (mg of antibody per kg weight of the subject) to about or equal to 100 mg/kg. In some aspects, an effective amount of an antibody provided therein is about or equal to 0.1 mg/kg, about or equal to 0.5 mg/kg, about or equal to 1 mg/kg, about or equal to 3 mg/kg, about or equal to 5 mg/kg, about or equal to 10 mg/kg, about or equal to 15 mg/kg, about or equal to 20 mg/kg, about or equal to 25 mg/kg, about or equal to 30 mg/kg, about or equal to 35 mg/kg, about or equal to 40 mg/kg, about or equal to 45 mg/kg, about or equal to 50 mg/kg, about or equal to 60 mg/kg, about or equal to 70 mg/kg, 80 mg/kg, 90 mg/kg, or 100 mg/kg. These amounts are meant to include amounts and ranges therein. In some aspects, “effective amount” also refers to the amount of an antibody provided herein to achieve a specified result (e.g., preventing, blocking, or inhibiting SLO binding to a host cell membrane).

The term “in combination” in the context of the administration of other therapies (e.g., other agents, antibiotic drugs, antifungal therapies) includes the use of more than one therapy (e.g., drug therapy and/or antibiotic therapy and/or antifungal therapy). Administration “in combination with” one or more further therapeutic agents includes simultaneous (e.g., concurrent) and consecutive administration in any order. The use of the term “in combination” does not restrict the order in which therapies are administered to a subject. By way of nonlimiting example, a first therapy (e.g., agent, such as antibodies or antibody fragments disclosed herein) may be administered before (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., agent) to a subject having or diagnosed with a necrotizing soft tissue infection.

In some aspects, the second therapy or agent that can be used in combination with drugs used to treat necrotizing soft tissue infections include but are not limited to imipenem, meropenem, ertapenem, piperacillin (tazobactam), tigecycline, penicillin, clindamycin, a fluoroquinolone or an aminoglycoside, vancomycin, daptomycin, linezolid, azithromycin, or erythromycin.

The combination of therapies (e.g., use of agents, including therapeutic agents) may be more effective than the additive effects of any two or more single therapy (e.g., have a synergistic effect). A synergistic effect is typically unexpected and cannot be predicted. For example, a synergistic effect of a combination of therapeutic agents frequently permits the use of lower dosages of one or more of the agents and/or less frequent administration of the agents to a patient. The ability to utilize lower dosages of therapeutics and antibiotic therapies and/or to administer the therapies less frequently reduces the potential for toxicity associated with the administration of the therapies to a subject without reducing the effectiveness of the therapies. In addition, a synergistic effect may result in improved efficacy of therapies in the treatment or alleviation of a necrotizing soft tissue infection. Also, a synergistic effect demonstrated by a combination of therapies (e.g., therapeutic agents) may avoid or reduce adverse or unwanted side effects associated with the use of any single therapy.

As used herein, the term “comprising” can include the aspects “consisting of” and “consisting essentially of.” “Comprising” can also mean “including but not limited to.”

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

As used herein, the term “determining” can refer to measuring or ascertaining a quantity or an amount or a change in activity. For example, determining the amount of a disclosed polypeptide, protein, gene or antibody in a sample as used herein can refer to the steps that the skilled person would take to measure or ascertain some quantifiable value of the polypeptide protein, gene or antibody in the sample. The art is familiar with the ways to measure an amount of the disclosed polypeptide, proteins, genes or antibodies in a sample.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, disorder, sickness, illness, complaint, affection, infection. In some aspects, the disease, disorder or condition can be a necrotizing soft tissue infection. In some aspects, the necrotizing soft tissue infection can be fasciitis, myositis or cellulitis.

As used herein, the term “streptolysin O” or “SLO” refers to a polypeptide (the terms “polypeptide” and “protein” are used interchangeably herein) or any native SLO that is secreted from, for example, Streptococcus, in any infected vertebrate source, including mammals such as primates (e.g., humans, cynomolgus monkey (cyno)), pets (e.g. dogs and cats), and livestock (e.g., cows and horses) unless otherwise indicated, and, in certain aspects, including various SLO isoforms, related SLO polypeptides, including SNP variants thereof. SLO is a haemolytic exotoxin secreted from various Streptococcus species belonging to a family of cholesterol-dependent cytolysins, and is oxygen labile. Examples of Streptococcus species that carry the genes for SLO include, but are not limited to, S. dysgalactiae and S. canis.

An exemplary amino acid sequence of human SLO is accession number AAT86323. An exemplary nucleotide of human SLO is accession number CP000003. In some aspects, the SLO amino acid sequence is encoded by the nucleotide sequence set forth in accession number CP000003. These sequences are conserved in multiple Streptococcus strains.

Abbreviations for the amino acid residues that comprise polypeptides and peptides described herein, and conservative substitutions for these amino acid residues are shown in Table 1 below. A polypeptide that contains one or more conservative amino acid substitutions or a conservatively modified variant of a polypeptide described herein refers to a polypeptide in which the original or naturally occurring amino acids are substituted with other amino acids having similar characteristics, for example, similar charge, hydrophobicity/hydrophilicity, side-chain size, backbone conformation, structure and rigidity, etc. Thus, these amino acid changes can typically be made without altering the biological activity, function, or other desired property of the polypeptide, such as its affinity or its specificity for antigen. In general, single amino acid substitutions in nonessential regions of a polypeptide do not substantially alter biological activity. Furthermore, substitutions of amino acids that are similar in structure or function are less likely to disrupt the polypeptides' biological activity.

TABLE 1
Amino Acid Residues and Examples of
Conservative Amino Acid Substitutions

| Original residue | |
|---|---|
| Three letter code and | Conservative |
| Single letter code | substitution(s) |
| Alanine (Ala) (A) | Gly; Ser |
| Arginine (Arg) (R) | Lys; His |
| Asparagine (Asn) (N) | Gln; His |
| Aspartic Acid (Asp) (D) | Glu; Asn |
| Cysteine (Cys) (C) | Ser; Ala |
| Glutamine (Gln) (Q) | Asn |
| Glutamic Acid (Glu) (E) | Asp; Gln |
| Glycine (Gly) (G) | Ala |
| Histidine (His) (H) | Asn; Gln |
| Isoleucine (Ile) (I) | Leu; Val |
| Leucine (Leu) (L) | Ile; Val |
| Lysine (Lys) (K) | Arg; His |
| Methionine (Met) (M) | Leu; Ile; Tyr |
| Phenylalanine (Phe) (F) | Tyr; Met; Leu |
| Proline (Pro) (P) | Ala |
| Serine (Ser) (S) | Thr |
| Threonine (Thr) (T) | Ser |
| Tryptophan (Trp) (W) | Tyr; Phe |
| Tyrosine (Tyr) (Y) | Trp; Phe |
| Valine (Val) (V) | Ile; Leu |

As used herein, the term “polypeptide” or “peptide” refers to a polymer of amino acids of three or more amino acids in a serial array, linked through peptide bonds. As used herein, the term “amino acid sequence” refers to a list of abbreviations, letters, characters or words representing amino acid residues. “Polypeptides” can be proteins, protein fragments, protein analogs, oligopeptides and the like. The amino acids that comprise the polypeptide may be naturally derived or synthetic. The polypeptide may be purified from a biological sample. For example, a SLO polypeptide or peptide may be composed of at least 3, 4, 5, 6, 7, 8, 9, 10, 11, 12, 13, 14, 15, 16, 17, 18, 19, 20, 21, 22, 23, 24, or 25 contiguous amino acids of SLO. In some aspects, the polypeptide has at least 25, 30, 35, 40, 45, 50, 55, 60, 65, 70, 75, 80, 85, 90, 95, 100, 105, 110, 115, 120, 125, 130, or 135, contiguous amino acids of SLO. In some aspects, the SLO polypeptide comprises at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 200 contiguous amino acid residues, at least 250 contiguous amino acid residues, at least 300 contiguous amino acid residues, at least 350 contiguous amino acid residues, at least 400 contiguous amino acid residues, at least 450 contiguous amino acid residues, at least 500 contiguous amino acid residues, at least 550 contiguous amino acid residues of the amino acid sequence of the SLO polypeptide. In some aspects, the SLO polypeptide or peptide may be composed of at least 574 contiguous amino acids. In some aspects, the SLO polypeptide or peptide may be composed of at least 596 contiguous amino acids. In some aspects, the SLO polypeptide or peptide will not include the signal sequence. In some aspects, the SLO polypeptide or peptide can include additional amino acid residues for purification and/or B-cell isolation.

In some aspects, the mutant SLO polypeptide sequence can comprise at least 596 contiguous amino acids. In some aspects, the signal sequence can be removed from the SLO mutant sequence. In some aspects, the SLO mutant sequence can comprise additional sequences for purification and/or B-cell isolation. In some aspects, the SLO mutant sequence can comprise at least 5, 10, 15, 20, 25, 30, 35, 40, 45, 50, 55, 56, or more contiguous amino acids for purification and/or B-cell isolation.

The wild type nucleotide SLO sequence is:

(SEQ ID NO: 149)
GAATCGAACAAACAAAACACTGCTAGTACAGAAACCACAACGACAAATG
AGCAACCAAAACCAGAAAGTAGTGAGCTAACTATCGAAAAAGCAGGTCA
GAAAATGGATGATATGCTTAACTCTAACGATATGATTAAGCTTGCTCCC
AAAGAAATGCCACTAGAATCTGCAGAAAAAGAAGAAAAAAAGTCAGAAG
ACAAAAAAAAGAGCGAAGAAGATCACACTGAAGAAATCAATGACAAGAT
TTATTCACTAAATTATAATGAGCTTGAAGTACTTGCTAAAAATGGTGAA
ACCATTGAAAATTTTGTTCCTAAAGAAGGCGTTAAGAAAGCTGATAAAT
TTATTGTCATTGAAAGAAAGAAAAAAAATATCAACACTACACCAGT
CGATATTTCC ATTATTGACT CTGTCACTGA
TAGGACCTATCCAGCAGCCC
TTCAGCTGGCTAATAAAGGTTTTACCGAAAACAAACCAGACGCGGTAGT
CACCAAGCGAAACCCACAAAAAATCCATATTGATTTACCAGGTATGGGA
GACAAAGCAACGGTTGAGGTCAATGACCCTACCTATGCCAATGTTTCAA
CAGCTATTGATAATCTTGTTAACCAATGGCATGATAATTATTCTGGTGG
TAATACGCTTCCTGCCAGAACACAATATACTGAATCAATGGTATATTCT
AAGTCACAGATTGAAGCAGCTCTAAATGTTAATAGCAAAATCTTAGATG
GTACTTTAGGCATTGATTTCAAGTCGATTTCAAAAGGTGAAAAGAAGGT
GATGATTGCAGCATACAAGCAAATTTTTTACACCGTATCAGCAAACCTT
CCTAATAATCCTGCGGATGTGTTTGATAAATCAGTGA
CCTTTAAAGATTTGCAACGAAAAGGTGTCAG
CAATGAAGCTCCGCCACTCTTTGTGAGTAACGTAGCCTAT
GGTCGAACTGTTTTTGTCAAACTAGAAACAAGTTCTAAAAGTAATGATG
TTGAAGCGGCCTTTAGTGCAGCTCTAAAAGGAACAGATGTTAAAACGAA
TGGAAAATACTC
TGATATCTTAGAAAATAGCTCATTTACAGCTGTCGTTTTAGGAGGAGAT
GCTGCAGAGCA CAATAAGGTG
GTCACAAAAGACTTTGATGTTATTAGAAACGTTATCAAAG
ACAATGCTACCTTCAGTAGAAAAAACCCAGCTTATCCTATTTCATACAC
CAGTGTTTTCCTTAAAAATAATAAAATTGCGGGTGTCAATAACAGAACT
GAATACGTTGAAACAACATCTACCGAGTACACTAGTGGAAAAATTAACC
TGTCTCATCAAGGCGCGTATGTTGCTCAATATGAAATCCTTTGGGATGA
AATCAATTATGATGACAAAGGAAAAGAAGTGATTACAAAACGACGTTGG
GACAACAACTGGTATAGTAAGACATCACCATTTAGCACAGTTATCCCAC
TAGGAGCTAATTCACGAAATATCCGTATCATGGCTAGAGAGGCCACCGG
CTTAGCTTGGGAATGGTGGCGAAAAGTGATCGACGAAAGAGATGTGAAA
CTATCTAAAGAAATCAATGTCAACATCTCAGGATC
AACCTTGAGCCCATATGGTTCGATTACTTATAAGTAGGA.

TABLE 2
Streptolysin O Mutant Sequences

| SEQ ID | | |
|---|---|---|
| NO: | Sequence | Description |
| 26 | accatggggggttctcatcatcatcatcatcatggtatggctagcatgactggtggac | Streptolysin O |
| | agcaaatgggtcgggatctgtacgacgatgacgataaggatcgatggggatcc | (SLO) from |
| | ggcctgaacgatatttttgaagcgcagaaaattgaatggcatgaaggtggtggcggt | Streptococcus |
| | tccgaatcgaacaaacaaaacactgctagtacagaaaccacaacgacaaatgagc | pyogenes; |
| | aaccaaaaccagaaagtagtgagctaactatcgaaaaagcaggtcagaaaatggat | italics |
| | g atatgcttaa ctctaacgatatgattaagc ttgctcccaa agaaatgcca | indicates start |
| | ctagaatctg cagaaaaaga agaaaaaaagtcagaagaca aaaaaaagag | codon; |
| | cgaagaagat cacactgaag aaatcaatga caagatttattcactaaatt | underline |
| | ataatgagct tgaagtactt gctaaaaatg gtgaaaccat | indicates part |
| | tgaaaattttgttcctaaag aaggcgttaa gaaagctgat aaatttattg | of the plasmid |
| | tcattgaaag aaagaaaaaaaatatcaaca ctacaccagt cgatatttcc | SLO is in; |
| | attattgact ctgtcactga taggacctatccagcagccc ttcagctggc | bold indicates |
| | taataaaggt tttaccgaaa acaaaccaga cgcggtagtcaccaagcgaa | mutation to |
| | acccacaaaa aatccatatt gatttaccag gtatgggaga | make SLO |
| | caaagcaacggttgaggtca atgaccctac ctatgccaat gtttcaacag | oxygen stable |
| | ctattgataa tcttgttaaccaatggcatg ataattattc tggtggtaat | (gc; C to A); |
| | acgcttcctg ccagaacaca atatactgaatcaatggtat attctaagtc | mutation made |
| | acagattgaa gcagctctaa atgttaatag caaaatcttagatggtactt | to generate |
| | taggcattga tttcaagtcg atttcaaaag gtgaaaagaa | SLO that is |
| | ggtgatgattgcagcataca agcaaatttt ttacaccgta | unable to bind |
| | tcagcaaaccttcctaataa tcctgcggatgtgtttgata aatcagtgac | to membrane |
| | ctttaaagat ttgcaacgaa aaggtgtcag caatgaagctccgccactct | cholesterol, |
| | ttgtgagtaacgtagcctat ggtcgaactg tttttgtcaa | T564G, L565G |
| | actagaaacaagttctaaaa gtaatgatgt tgaagcggcc tttagtgcag | (AACCTTG to |
| | ctctaaaaggaacagatgttaaaacgaatg gaaaatactc tgatatctta | GGTGGC); |
| | gaaaatagct catttacagc tgtcgttttaggaggagatg ctgcagagca | underline and |
| | caataaggtg gtcacaaaag actttgatgt tattagaaacgttatcaaag | italics indicate |
| | acaatgctac cttcagtaga aaaaacccag cttatcctat | stop codon. |
| | ttcatacaccagtgttttcc ttaaaaataa taaaattgcg ggtgtcaata | |
| | acagaactga atacgttgaaacaacatcta ccgagtacac tagtggaaaa | |
| | attaacctgt ctcatcaagg cgcgtatgttgctcaatatgaaatcctttg | |
| | ggatgaaatc aattatgatgacaaaggaaaagaagtgatt | |
| | acaaaacgacgttgggacaacaactggtatagtaagacatcaccatttagcacagtt | |
| | atcccactaggagctaattcacgaaatatccgtatcatggctagagaggccaccggc | |
| | ttagcttgggaatggtggcgaaaagtgatcgacgaaagagatgtgaaactatctaaa | |
| | gaaatcaatgtcaacatctcaggatcaaccttgagcccatatggttcgattacttataa | |
| | gtagga | |
| 27 | MGGSHHHHHHGMASMTGGQQMGRDL | Amino acid |
| | YDDDDKDRWGSGLNDIFEAQKIEWHE | sequence SLO |
| | GGGGSESNKQNTASTETTTTNEQPKPE | used for antigen |
| | SSELTIEKAGQKMDDMLNSNDMIKLA | baiting; underline |
| | PKEMPLESAEKEEKKSEDKKKSEEDHT | indicates plasmid |
| | EEINDKIYSLNYNELEVLAKNGETIEN | sequence; |
| | FVPKEGVKKADKFIVIERKKKNINTTP | italics indicate |
| | VDISIIDSVTDRTYPAALQLANKGFTE | the AviTag + GGG |
| | NKPDAVVTKRNPQKIHIDLPGMGDKA | S sequence that |
| | TVEVNDPTYANVSTAIDNLVNQWHDN | was added for |
| | YSGGNTLPARTQYTESMVYSKSQIEAA | antigen |
| | LNVNSKILDGTLGIDFKSISKGEKKVM | baiting; bold |
| | IAAYKQIFYTVSANLPNNPADVFDKSV | indicates |
| | TFKDLQRKGVSNEAPPLFVSNVAYGR | mutation made |
| | TVFVKLETSSKSNDVEAAFSAALKGTD | to generate |
| | VKTNGKYSDILENSSFTAVVLGGDAA | SLO that is |
| | EHNKVVTKDFDVIRNVIKDNATFSRK | unable to bind |
| | NPAYPISYTSVFLKNNKIAGVNNRTEY | membrane |
| | VETTSTEYTSGKINLSHQGAYVAQYEI | cholesterol; |
| | LWDEINYDDKGKEVITKRRWDNNWYS | HHHHHH |
| | KTSPFSTVIPLGANSRNIRIMAREATGL | (SEQ ID NO: |
| | AWEWWRKVIDERDVKLSKEINVNISG | 152) is the His |
| | SGGSPYGSITYK | tag sequence; |
| | | bold A is the |
| | | mutation to |
| | | make SLO |
| | | oxygen stable |
| | | (C to A); bold |
| | | GG is the |
| | | mutation made |
| | | to generate |
| | | SLO that is |
| | | unable to bind |
| | | membrane |
| | | cholesterol, |
| | | T564G, L565G |
| | | (AACCTTG to |
| | | GGTGGC) |

SEQ ID NO: 26 is an example of a mutant SLO nucleotide sequence. SEQ ID NO: 26 includes mutations, for example, a mutation was made to make SLO oxygen stable (e.g, TGC to GCC. Also, SEQ ID NO: 26 includes a mutation to generate SLO that is unable to bind membrane cholesterol (e.g., AACCTTG to GGTGGC). SEQ ID NO: 148 is CATCATCATCATCATCAT, and can encode a His tag sequence. SEQ ID NO: 150 is GGCCTGAACGATATTTTTGAAGCGCAGAAAATTGAATGGCATGAAGGTGGTGGC GGTTCC, and can encode the AgiTag+GGGGS. SEQ ID NO: 151 is

GAATCGAACAAACAAAACACTGCTAGTACAGAAACCACAACGACAAATG
AGCAACCAAAACCAGAAAGTAGTGAGCTAACTATCGAAAAAGCAGGTCA
GAAAATGGATGATATGCTTAACTCTAACGATATGATTAAGCTTGCTCCC
AAAGAAATGCCACTAGAATCTGCAGAAAAAGAAGAAAAAAAGTCAGAAG
ACAAAAAAAAGAGCGAAGAAGATCACACTGAAGAAATCAATGACAAGAT
TTATTCACTAAATTATAATGAGCTTGAAGTACTTGCTAAAAATGGTGAA
ACCATTGAAAATTTTGTTCCTAAAGAAGGCGTTAAGAAAGCTGATAAAT
TTATTGTCATTGAAAGAAAGAAAAAAAATATCAACACTACACCAGTCGA
TATTTCCATTATTGACTCTGTCACTGATAGGACCTATCCAGCAGCCCTT
CAGCTGGCTAATAAAGGTTTTACCGAAAACAAACCAGACGCGGTAGTCA
CCAAGCGAAACCCACAAAAAATCCATATTGATTTACCAGGTATGGGAGA
CAAAGCAACGGTTGAGGTCAATGACCCTACCTATGCCAATGTTTCAACA
GCTATTGATAATCTTGTTAACCAATGGCATGATAATTATTCTGGTGGTA
ATACGCTTCCTGCCAGAACACAATATACTGAATCAATGGTATATTCTAA
GTCACAGATTGAAGCAGCTCTAAATGTTAATAGCAAAATCTTAGATGGT
ACTTTAGGCATTGATTTCAAGTCGATTTCAAAAGGTGAAAAGAAGGTGA
TGATTGCAGCATACAAGCAAATTTTTTACACCGTATCAGCAAACCTTCC
TAATAATCCTGCGGATGTGTTTGATAAATCAGTGACCTTTAAAGATTTG
CAACGAAAAGGTGTCAGCAATGAAGCTCCGCCACTCTTTGTGAGTAACG
TAGCCTATGGTCGAACTGTTTTTGTCAAACTAGAAACAAGTTCTAAAAG
TAATGATGTTGAAGCGGCCTTTAGTGCAGCTCTAAAAGGAACAGATGTT
AAAACGAATGGAAAATACTCTGATATCTTAGAAAATAGCTCATTTACAG
CTGTCGTTTTAGGAGGAGATGCTGCAGAGCACAATAAGGTGGTCACAAA
AGACTTTGATGTTATTAGAAACGTTATCAAAGACAATGCTACCTTCAGT
AGAAAAAACCCAGCTTATCCTATTTCATACACCAGTGTTTTCCTTAAAA
ATAATAAAATTGCGGGTGTCAATAACAGAACTGAATACGTTGAAACAAC
ATCTACCGAGTACACTAGTGGAAAAATTAACCTGTCTCATCAAGGCGCG
TATGTTGCTCAATATGAAATCCTTTGGGATGAAATCAATTATGATGACA
AAGGAAAAGAAGTGATTACAAAACGACGTTGGGACAACAACTGGTATAG
TAAGACATCACCATTTAGCACAGTTATCCCACTAGGAGCTAATTCACGA
AATATCCGTATCATGGCTAGAGAGGCCACCGGCTTAGCTTGGGAATGGT
GGCGAAAAGTGATCGACGAAAGAGATGTGAAACTATCTAAAGAAATCAA
TGTCAACATCTCAGGATCAACCTTGAGCCCATATGGTTCGATTACTTAT
AAGTAGGA

corresponds to the SLO nucleotide sequence.

SEQ ID NO: 27 is an example of a mutant SLO polypeptide sequence. In some aspects, the T564G, L565G (Farrand et al., Proc Natl Acad Sci USA, 2010, Mar. 2: 107(9):4341) and C499A (Pinkney et al., Infect Immun, 1995 July; 63(7):2776-9) mutations of are incorporated into the sequence of SEQ ID NO: 27 at the corresponding positions of the SLO sequence of SEQ ID NO: 27. SEQ ID NO: 27 includes mutations, for example, a T to G mutation at position 586, a L to G mutation at position 587, and a C to A mutation at position 555. A T to G mutation at position 586 and a L to G mutation at position 587 mutations were incorporated to generate SLO that is unable to bind membrane cholesterol. The C to A mutation at position 555 was incorporated to generate SLO to make the protein oxygen stable.

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

As used herein, the term “analog” refers to a polypeptide that possesses a similar or identical function as a reference polypeptide but does not necessarily comprise a similar or identical amino acid sequence of the reference polypeptide, or possess a similar or identical structure of the reference polypeptide. The reference polypeptide may be a native SLO polypeptide, a fragment of a native SLO polypeptide, or an anti-SLO antibody that is capable of binding to SLO. A polypeptide that has a similar amino acid sequence with a reference polypeptide refers to a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of the reference polypeptide, which can be a SLO polypeptide or an antibody or antibody fragment disclosed herein. A polypeptide with similar structure to a reference polypeptide refers to a polypeptide that has a secondary, tertiary, or quaternary structure similar to that of the reference polypeptide, which can be a SLO polypeptide or an antibody or antibody fragment disclosed herein. The structure of a polypeptide can determined by methods known to those skilled in the art, including, but not limited to, X-ray crystallography, nuclear magnetic resonance (NMR), and crystallographic electron microscopy.

The term “fragment” can refer to a portion (e.g., at least 5, 10, 25, 50, 100, 125, 150, 200, 250, 300, 350, 400 or 500, etc. amino acids or nucleic acids) of a protein or nucleic acid molecule that is substantially identical to a reference protein or nucleic acid and retains the biological activity of the reference. In some aspects, the fragment or portion retains at least 50%, 75%, 80%, 85%, 90%, 95% or 99% of the biological activity of the reference protein or nucleic acid described herein. Further, a fragment of a referenced peptide can be a continuous or contiguous portion of the referenced polypeptide (e.g., a fragment of a peptide that is ten amino acids long can be any 2-9 contiguous residues within that peptide).

As used herein, the term “variant” when used in relation to a native SLO polypeptide or to an anti-SLO antibody refers to a polypeptide or an anti-SLO antibody having one or more amino acid sequence substitutions, deletions, and/or additions as compared to a native or unmodified SLO sequence or anti-SLO antibody sequence. For example, a SLO polypeptide or to an anti-SLO antibody refers to a polypeptide or an anti-SLO antibody having one or more amino acid sequence substitutions, deletions, and/or additions as compared to a native or unmodified SLO sequence or anti-SLO antibody sequence can have about 1 to about 25, about 1 to about 20, about 1 to about 15, about 1 to about 10, or about 1 to about 5 amino acid sequence substitutions, deletions, and/or additions as compared to a native or unmodified SLO sequence or anti-SLO antibody sequence. For example, SEQ ID NO: 27 is an example of a SLO variant. A SLO variant can result from one or more (such as, for example, about 1 to about 60, about 1 to about 55, about 1 to about 50, about 1 to about 45, about 1 to about 40, about 1 to about 35, about 1 to about 30, about 1 to about 25, about 1 to about 20, about 1 to about 15, about 1 to about 10, or about 1 to about 5) changes to an amino acid sequence of a native SLO. Also by way of example, a variant of an anti-SLO antibody can result from one or more (such as, for example, about 1 to about 25, about 1 to about 20, about 1 to about 15, about 1 to about 10, or about 1 to about 5 changes to an amino acid sequence of a native or previously unmodified anti-SLO antibody. Variants can be naturally occurring, such as allelic or splice variants, or can be artificially constructed. Polypeptide variants can be prepared from the corresponding nucleic acid molecules encoding the variants. In some aspects, a variant can be any of the SLO polypeptides described herein.

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions can include those within the following groups: Ser, Thr, and Cys; Leu, Ile, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, a variant may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂)ₙCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among residues with neutral hydrophilic side chains (e.g., Cys, Ser, Thr, Asn and Gln), among residues with acidic side chains (e.g., Asp, Glu), among amino acids with basic side chains (e.g., His, Lys, and Arg), or among residues with aromatic side chains (e.g., Trp, Tyr, and Phe). As known in the art, conservative substitution usually does not cause significant change in the protein conformational structure, and therefore could retain the biological activity of a protein.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (e.g., an “algorithm”). Methods that may be used to calculate the identity of the aligned nucleic acids or polypeptides include those described in Lesk, A. M., ed., 1988, Computational Molecular Biology, New York: Oxford University Press; Smith, D. W., ed., 1993, Biocomputing Informatics and Genome Projects, New York: Academic Press; Griffin, A. M., et al., 1994, Computer Analysis of Sequence Data, Part I, New Jersey: Humana Press; von Heinje, G., 1987, Sequence Analysis in Molecular Biology, New York: Academic Press; Gribskov, M. et al., 1991, Sequence Analysis Primer, New York: M. Stockton Press; and Carillo et al., 1988, Applied Math., 48:1073.

In calculating percent identity, the sequences being compared can be aligned in a way that gives the largest match between the sequences. An example of a computer program that can be used to determine percent identity is the GCG program package, which includes GAP (Devereux et al., 1984, Nucl. Acid Res., 12:387; Genetics Computer Group, University of Wisconsin, Madison, WI), which is a computer algorithm used to align the two polypeptides or polynucleotides to determine their percent sequence identity. The sequences can be aligned for optimal matching of their respective amino acid or nucleotide sequences (the “matched span” as determined by the algorithm). A gap opening penalty (which is calculated as 3 times the average diagonal, wherein the “average diagonal” is the average of the diagonal of the comparison matrix being used, and the “diagonal” is the score or number assigned to each perfect amino acid match by the particular comparison matrix; and a gap extension penalty (which is usually 1/10 times the gap opening penalty), as well as a comparison matrix such as PAM 250 or BLOSUM 62, are used in conjunction with the algorithm. In some aspects, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. USA 89:10915-10919 for the BLOSUM 62 comparison matrix) is also used by the algorithm. Exemplary parameters for determining percent identity for polypeptides or nucleotide sequences using the GAP program include the following: (i) Algorithm: Needleman et al., 1970, J. Mol. Biol., 48:443-453; (ii) Comparison matrix: BLOSUM 62 from Henikoff et al., Id.; (iii) Gap Penalty: 12 (but with no penalty for end gaps); (iv) Gap Length Penalty: 4; and (v) Threshold of Similarity: 0.

Certain alignment schemes for aligning two amino acid sequences can result in matching only a short region of the two sequences, and this small aligned region can have very high sequence identity even though there is no significant relationship between the two full-length sequences. Accordingly, the selected alignment method (e.g., the GAP program) can be adjusted if so desired to result in an alignment that spans a representative number of amino acids, for example, at least 50 contiguous amino acids, of the target polypeptide.

Percent (%) amino acid sequence identity with respect to a reference polypeptide sequence is defined as the percentage of amino acid residues in a candidate sequence that is identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill of the practitioner in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, the term “derivative” refers to a polypeptide that comprises an amino acid sequence of a reference polypeptide that has been altered by the introduction of amino acid residue substitutions, deletions or additions. The reference polypeptide can be a native SLO polypeptide or an anti-SLO antibody. The term “derivative” as used herein also refers to a SLO polypeptide or an anti-SLO antibody that has been chemically modified, e.g., by the covalent attachment of any type of molecule to the polypeptide. For example, a SLO polypeptide or an anti-SLO antibody can be chemically modified, e.g., by glycosylation, acetylation, pegylation, phosphorylation, amidation, derivatization by known protecting/blocking groups, proteolytic cleavage, linkage to a cellular ligand, linkage to a peptide or protein tag molecule, or other protein, etc. The derivatives are modified in a manner that is different from the naturally occurring or starting peptide or polypeptides, either in the type or location of the molecules attached. Derivatives may further include deletion of one or more chemical groups which are naturally present on the peptide or polypeptide. A derivative of a SLO polypeptide or an anti-SLO antibody may be chemically modified by chemical modifications using techniques known to those of skill in the art, including, but not limited to, specific chemical cleavage, acetylation, formulation, metabolic synthesis by tunicamycin, etc. Further, a derivative of a SLO polypeptide or an anti-SLO antibody can contain one or more non-classical amino acids. A polypeptide derivative possesses a similar or identical function as the reference polypeptide, which can be a SLO polypeptide or an anti-SLO antibody described herein, especially an anti-SLO monoclonal antibody.

The term “fusion protein” as used herein refers to a polypeptide that includes amino acid sequences of at least two heterologous polypeptides. The term “fusion” when used in relation to a SLO polypeptide or to an anti-SLO antibody refers to the joining, fusing, or coupling of a SLO polypeptide or an anti-SLO antibody, variant and/or derivative thereof, with a heterologous peptide or polypeptide. In some aspects, the fusion protein retains the biological activity of the SLO polypeptide or the anti-SLO antibody. In some aspects, the fusion protein includes a SLO antibody VH region, VL region, VH CDR (one, two or three VH CDRs), and/or VL CDR (one, two or three VL CDRs) coupled, fused, or joined to a heterologous peptide or polypeptide, wherein the fusion protein binds to an epitope on a SLO protein or peptide. Fusion proteins may be prepared via chemical coupling reactions as practiced in the art, or via molecular recombinant technology.

As used herein, the term “composition” refers to a product containing specified component ingredients (e.g., a polypeptide or an antibody provided herein) in, optionally, specified or effective amounts, as well as any desired product which results, directly or indirectly, from the combination or interaction of the specific component ingredients in, optionally, the specified or effective amounts.

As used herein, the term “carrier” includes pharmaceutically acceptable carriers, excipients, diluents, vehicles, or stabilizers that are nontoxic to the cell or mammal being exposed thereto at the dosages and concentrations employed. Often, the physiologically acceptable carrier is an aqueous pH buffered solution. Examples of physiologically acceptable carriers include buffers such as phosphate, citrate, succinate, and other organic acids; antioxidants including ascorbic acid; low molecular weight (e.g., less than about 10 amino acid residues) polypeptides; proteins, such as serum albumin, gelatin, or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose, sucrose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. The term “carrier” can also refer to a diluent, adjuvant (e.g., Freund's adjuvant, complete or incomplete), excipient, or vehicle with which the therapeutic is administered. Such carriers, including pharmaceutical carriers, can be sterile liquids, such as water and oils, including those of petroleum, animal, vegetable or synthetic origin, such as peanut oil, soybean oil, mineral oil, sesame oil and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include, without limitation, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Compositions can take the form of solutions, suspensions, emulsion, tablets, pills, capsules, powders, sustained-release formulations and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, (1990) Mack Publishing Co., Easton, PA. Compositions, including pharmaceutical compounds, can contain a therapeutically effective amount of an anti-SLO antibody in isolated or purified form, together with a suitable amount of carrier so as to provide the form for proper administration to the subject (e.g., patient). The composition or formulation should suit the mode of administration.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, PA, which is hereby incorporated by reference in its entirety.

As used herein, the term “pharmaceutically acceptable” or “pharmacologically acceptable” refers to molecular entities, formulations and compositions that do not produce an adverse, allergic, or other untoward or unwanted reaction when administered, as appropriate, to an animal, such as a human. The preparation of a pharmaceutical composition comprising an antibody or additional active ingredient are known to those of skill in the art in light of the present disclosure, as exemplified by Remington's Pharmaceutical Sciences, Id. Moreover, for animal (e.g., human) administration, it will be understood that preparations should meet sterility, pyrogenicity, general safety, and purity standards as required by a regulatory agency of the Federal or a state government, such as the FDA Office of Biological Standards or as listed in the U.S. Pharmacopeia, European Pharmacopeia, or other generally recognized Pharmacopeia for use in animals, and more particularly, in humans.

The term “pharmaceutical formulation” refers to a preparation which is in such form as to permit the biological activity of the active ingredient (e.g., antibodies or antibody fragments disclosed herein) to be effective, and which contains no additional components that would be are unacceptably toxic to a subject to whom the formulation would be administered. Such a formulation can be sterile, i.e., aseptic or free from all living microorganisms and their spores, etc.

The term “package insert” is used to refer to instructions customarily included in commercial packages of therapeutic products, that contain information about the indications, usage, dosage, administration, contraindications and/or warnings concerning the use of such therapeutic products.

The terms “antibody,” “immunoglobulin,” and “Ig” are used interchangeably herein in a broad sense and specifically cover, for example, individual antibodies or antibody fragments disclosed herein, such as the monoclonal antibodies described herein, (including agonist, antagonist, neutralizing antibodies, full length or intact monoclonal antibodies, peptide fragments of antibodies that maintain antigen binding activity); anti-SLO antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain anti-SLO antibodies, and fragments of anti-SLO, as described herein. An antibody can be human, humanized, chimeric and/or affinity matured. An antibody may be from other species, for example, mouse, rat, rabbit, etc. The term “antibody” is intended to include a polypeptide product of B cells within the immunoglobulin class of polypeptides that is able to bind to a specific molecular antigen. An antibody is typically composed of two identical pairs of polypeptide chains, wherein each pair has one heavy chain (about 50-70 kDa) and one light chain (about 25 kDa); and wherein the amino-terminal portion of the heavy and light chains includes a variable region of about 100 to about 130 or more amino acids and the carboxy-terminal portion of each chain includes a constant region (See, Borrebaeck (ed.), 1995, Antibody Engineering, Second Ed., Oxford University Press.; Kuby, 1997 Immunology, Third Ed., W.H. Freeman and Company, New York). In some aspects, the specific molecular antigen bound by an antibody provided herein includes a SLO polypeptide, a SLO peptide fragment, or a SLO epitope. An antibody or a peptide fragment thereof that binds to a SLO antigen can be identified, for example, by immunoassays, BIAcore, or other techniques known to those of skill in the art. An antibody or a fragment thereof binds specifically to a SLO antigen when it binds to a SLO antigen with higher affinity than to any cross-reactive antigen as determined using experimental techniques, such as radioimmunoassays (RIA) and enzyme linked immunosorbent assays (ELISAs). Typically, a specific or selective binding reaction will be at least twice background signal or noise, and more typically more than 5-10 times background signal or noise. See, e.g., Paul, ed., 1989, Fundamental Immunology Second Edition, Raven Press, New York at pages 332-336 for a discussion regarding antibody specificity.

Antibodies provided herein include, but are not limited to, synthetic antibodies, monoclonal antibodies, recombinantly produced antibodies, multispecific antibodies (including bi-specific antibodies), human antibodies, humanized antibodies, camelized antibodies, chimeric antibodies, intrabodies, anti-idiotypic (anti-Id) antibodies, and functional fragments (e.g., antigen-binding fragments such as SLO binding fragments) of any of the above. A binding fragment refers to a portion of an antibody heavy or light chain polypeptide, such as a peptide portion, that retains some or all of the binding activity of the antibody from which the fragment is derived. Non-limiting examples of functional fragments (e.g., antigen-binding fragments such as SLO binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (sdFv), Fd fragments, Fv fragments, diabodies, triabodies, tetrabodies and minibodies. In particular, antibodies provided herein include immunoglobulin molecules and immunologically active portions of immunoglobulin molecules, for example, antigen binding domains or molecules that contain an antigen-binding site that binds to a SLO antigen, (e.g., one or more complementarity determining regions (CDRs) of an anti-SLO antibody). Description of such antibody fragments can be found in, for example, Harlow and Lane, 1989, Antibodies: A Laboratory Manual, Cold Spring Harbor Laboratory, New York; Myers (ed.), Molec. Biology and Biotechnology: A Comprehensive Desk Reference, New York: VCH Publisher, Inc.; Huston et al., 1993, Cell Biophysics, 22:189-224; Pluckthun and Skerra, 1989, Meth. Enzymol., 178:497-515 and in Day, E. D., 1990, Advanced Immunochemistry, Second Ed., Wiley-Liss, Inc., New York, NY. The antibodies provided herein can be of any type (e.g., IgG, IgE, IgM, IgD, IgA and IgY), any class (e.g., IgG1, IgG2, IgG3, IgG4, IgA1 and IgA2), or any subclass (e.g., IgG2a and IgG2b) of immunoglobulin molecule. Anti-SLO antibodies can be agonistic antibodies or antagonistic antibodies. In some aspects, the anti-SLO antibodies can be fully human, such as fully human monoclonal anti-SLO antibodies. In some aspects, the anti-SLO antibodies can be humanized, such as humanized monoclonal anti-SLO antibodies. In some aspects, the antibodies provided herein can be IgG antibodies, or a class (e.g., human IgG1 or IgG4) or subclass thereof, in particular, IgG1 subclass antibodies.

A four-chain antibody unit is a heterotetrameric glycoprotein composed of two identical light (L) chains and two identical heavy (H) chains. In the case of IgGs, the molecular weight of the four-chain (unreduced) antibody unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. At the N-terminus, each H chain has a variable domain (V_{H}) followed by three constant domains (C_{H}) for each of the α and γ chains and four C_{H} domains for p and E isotypes. Each L chain has at the N-terminus, a variable domain (V_{L}) followed by a constant domain (C_{L}) at its carboxy terminus. The V_{L} domain is aligned with the V_{H} domain, and the C_{L} domain is aligned with the first constant domain of the heavy chain (C_{H1}). Particular amino acid residues are believed to form an interface between the light chain and heavy chain variable domains. The pairing of a V_{H} and V_{L} together forms a single antigen-binding site, although certain V_{H} and V_{L} domains can bind antigen without pairing with a V_{L} or V_{H} domain, respectively. The basic structure of immunoglobulin molecules is understood by those having skill in the art. For example, the structure and properties of the different classes of antibodies may be found in Terr, Abba I. et al., 1994, Basic and Clinical Immunology, 8th edition, Appleton & Lange, Norwalk, CT, page 71 and Chapter 6.

A “single-chain variable fragment (scFv)” means a protein comprising the variable regions of the heavy and light chains of an antibody. A scFv can be a fusion protein comprising a variable heavy chain, a linker, and a variable light chain. In some aspects, the linker can be a short, flexible fragment that can be about 8 to 20 amino acids in length. For example, (G4S)n can be used (n=1, 2, 3 or 4).

A “fragment antigen-binding fragment (Fab)” is a region of an antibody that binds to antigen. An Fab comprises constant and variable regions from both heavy and light chains.

A “CDR” or complementarity determining region is a region of hypervariability interspersed within regions that are more conserved, termed “framework regions” (FR).

As used herein, the term “antigen” or “target antigen” is a predetermined molecule to which an antibody can selectively bind. A target antigen can be a polypeptide, peptide, carbohydrate, nucleic acid, lipid, hapten, or other naturally occurring or synthetic compound. In some aspects, a target antigen can be a small molecule. In some aspects, the target antigen can a polypeptide or peptide, e.g., SLO.

As used herein, the term “antigen binding fragment,” “antigen binding domain,” “antigen binding region,” and similar terms refer to that portion of an antibody which includes the amino acid residues that interact with an antigen and confer on the antibody as binding agent its specificity and affinity for the antigen (e.g., the CDRs of an antibody are antigen binding regions). The antigen binding region can be derived from any animal species, such as rodents (e.g., rabbit, rat, or hamster) and humans. In some aspects, the antigen binding region can be of human origin.

An “isolated” antibody is substantially free of cellular material or other contaminating proteins from the cell or tissue source and/or other contaminant components from which the antibody is derived, or is substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of an antibody that have less than about 30%, 25%, 20%, 15%, 10%, 5%, or 1% (by dry weight) of heterologous protein (also referred to herein as a “contaminating protein”). In some aspects, when the antibody is recombinantly produced, it is substantially free of culture medium, e.g., culture medium represents less than about 20%, 15%, 10%, 5%, or 1% of the volume of the protein preparation. In some aspects, when the antibody is produced by chemical synthesis, it is substantially free of chemical precursors or other chemicals, for example, it is separated from chemical precursors or other chemicals which are involved in the synthesis of the protein. Accordingly such preparations of the antibody have less than about 30%, 25%, 20%, 15%, 10%, 5%, or 1% (by dry weight) of chemical precursors or compounds other than the antibody of interest. Contaminant components can also include, but are not limited to, materials that would interfere with therapeutic uses for the antibody, and can include enzymes, hormones, and other proteinaceous or nonproteinaceous solutes. In some aspects, the antibody is purified (1) to greater than or equal to 95% by weight of the antibody, as determined by the Lowry method (Lowry et al., 1951, J. Bio. Chem., 193: 265-275), such as 95%, 96%, 97%, 98%, or 99%, by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. Isolated antibody also includes the antibody in situ within recombinant cells since at least one component of the antibody's natural environment will not be present. An isolated antibody is typically prepared by at least one purification step. In some aspects, the antibodies provided herein are isolated.

The term “monoclonal antibody” (monoclonal antibody) refers to an antibody, or population of like antibodies, obtained from a population of substantially homogeneous antibodies, and is not to be construed as requiring production of the antibody by any particular method, including but not limited to, monoclonal antibodies can be made by the hybridoma method first described by Kohler and Milstein (Nature, 256: 495-497, 1975), or by recombinant DNA methods.

As used herein, the term “binds” or “binding” refers to an interaction between molecules including, for example, to form a complex. Illustratively, such interactions embrace non-covalent interactions, including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site of an antibody and its epitope on a target (antigen) molecule, such as SLO, is the affinity of the antibody or functional fragment for that epitope. The ratio of association (kₒₙ) to dissociation (k_{off}) of an antibody to a monovalent antigen (kₒₙ/k_{off}) is the association constant Kₐ, which is a measure of affinity. The value of K varies for different complexes of antibody and antigen and depends on both kₒₙ and k_{off}. The association constant Kₐ for an antibody provided herein may be determined using any method provided herein or any other method known to those skilled in the art. The affinity at one binding site does not always reflect the true strength of the interaction between an antibody and an antigen. When complex antigens containing multiple, repeating antigenic determinants come into contact with antibodies containing multiple binding sites, the interaction of antibody with antigen at one site will increase the probability of an interaction at a second binding site. The strength of such multiple interactions between a multivalent antibody and antigen is called the avidity. The avidity of an antibody can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively.

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{d}). Affinity can be measured by common methods known in the art, including those described herein. Low-affinity antibodies generally bind antigen slowly and tend to dissociate readily, while high-affinity antibodies generally bind antigen faster and tend to remain bound longer to antigen. A variety of methods for measuring binding affinity are known in the art, any of which may be used for purposes of the present disclosure. Specific illustrative aspects include the following: In some aspects, the “K_{d}” or “K_{d} value” is measured by assays known in the art, for example, by a binding assay. The K_{d} can be measured in a radiolabeled antigen binding assay (RIA), for example, performed with the Fab portion of an antibody of interest and its antigen (Chen, et al., 1999, J. Mol. Biol., 293:865-881) or in an enzyme-linked immunosorbent assay (ELISA). The K_{d} or K_{d} value may also be measured by using surface plasmon resonance (SPR) assays (by BIAcore) using, for example, a BIAcore™-2000 or a BIAcore™-3000 (BIAcore, Inc., Piscataway, NJ), or by biolayer interferometry (BLI) using, for example, the OctetQK384 system (ForteBio, Menlo Park, CA), or by quartz crystal microbalance (QCM) technology. An “on-rate” or “rate of association” or “association rate” or “kₒₙ” can also be determined with the same surface plasmon resonance or biolayer interferometry techniques described above, using, for example, a BIAcore™-2000 or a BIAcore™-3000 (BIAcore, Inc., Piscataway, NJ), or the OctetQK384 system (ForteBio, Menlo Park, CA).

Disclosed herein are anti-SLO antibodies, antibodies that specifically bind to SLO, antibodies that are specific for SLO, antibodies that specifically bind to a SLO epitope, antibodies that selectively bind to a SLO epitope, and antibodies that preferentially binds to SLO. The terms “anti-SLO antibody,” “an antibody that specifically binds to SLO,” or “antibody that is specific for SLO,” “antibodies that specifically bind to a SLO epitope,” “an antibody that selectively binds to SLO,” “antibodies that selectively bind to a SLO epitope,” “an antibody that preferentially binds to SLO”, and analogous terms refer to antibodies capable of binding SLO, i.e., WT SLO, with sufficient affinity and specificity, particularly compared with mutants of SLO.

By “specifically binds” is meant that an antibody recognizes and physically interacts with its cognate antigen (for example, SLO) and does not significantly recognize and interact with other antigens; such an antibody may be a polyclonal antibody or a monoclonal antibody, which are generated by techniques that are well known in the art.

“Preferential binding” of the antibodies and antibody fragments as provided herein may be determined or defined based on the quantification of fluorescence intensity of the antibodies' binding to SLO, i.e., SLO polypeptide versus an appropriate control, such as binding to variant SLO, for example, molecularly engineered cells, cell lines or tumor cell isolates. Preferential binding of an anti-SLO antibody (e.g., any of the antibodies and antibody fragments as disclosed herein) as described to a SLO can be indicated by a measured fluorescent binding intensity (MFI) value, as assessed by cell flow cytometry, of at least 2-fold, at least 3-fold, at least 4-fold, at least 5-fold, at least 6-fold, at least 7-fold, at least 8-fold, at least 10-fold, at least 15-fold, at least 20-fold or greater, as compared with binding of the antibody to a mutant SLO polypeptide, wherein the antibody to be assayed is directly or indirectly detectable by a fluorescent label or marker, such as FITC. In some aspects, the antibody to be assayed is directly labeled with a fluorescent marker, such as FITC. In some aspects, an anti-SLO antibody that preferentially or selectively binds SLO exhibits an MFI value of from 1.5-fold to 25-fold, or from 2-fold to 20-fold, or from 3-fold to 15-fold, or from 4-fold to 8-fold, or from 2-fold to 10-fold, or from 2-fold to 5-fold or more greater than the MFI value of the same antibody for binding a SLO or a SLO variant. Fold-fluorescence intensity values between and equal to all of the foregoing are intended to be included. In some aspects, the anti-SLO antibodies specifically and preferentially bind to a SLO polypeptide, such as a SLO antigen, peptide fragment, or epitope. An antibody that specifically binds to SLO, (e.g., wild type or a variant SLO) can bind to the extracellular domain (ECD) or a peptide derived from the ECD of SLO. An antibody that specifically binds to an SLO antigen (e.g., SLO) can be cross-reactive with related antigens (e.g., cholesterol-dependent cytolysins). In some aspects, an antibody that specifically binds to a SLO antigen does not cross-react with other antigens. An antibody that specifically binds to a SLO antigen can be identified, for example, by immunofluorescence binding assays, immunohistochemistry assay methods, immunoassay methods, Biacore, or other techniques known to those of skill in the art.

In some aspects, an antibody that binds to SLO, as described herein, has a dissociation constant (K_{d}) of less than or equal to 80 pM, 75 pM, 70 pM, 65 pM, 60 pM, 55 pM, 50 pM, 45 pM, 40 pM, 35 pM, 30 pM, 25 pM, 20 pM, 15 pM, 10 pM, 9 pM, 8 pM, 7 pM, 6 pM, 5 pM, 4 pM, 3 pM, 2 pM, 1 pM, or 0.5 pM, and/or is greater than or equal to 0.1 pM. In some aspects, an antibody that binds to SLO, as described herein, has a dissociation constant (K_{d}) of less than or equal to 80 pM, 75 pM, 70 pM, 65 pM, 60 pM, 55 pM, 50 pM, 49 pM, 48 pM, 47 pM, 46 pM, 45 pM, 44 pM, 43 pM, 42 pM, 41 pM, 40 pM and/or is greater than or equal to 40 nM. In some aspects, an antibody that binds to SLO, as described herein, has a K_{d} of less than or equal to 100 fM, 75 fM, 50 fM, 25 fM, 10 fM, 9 pM, 8 pM, 7 pM, 6 pM, 5 pM, 4 pM, 3 pM, 2 pM, 1 pM, or 0.5 pM, and/or is greater than or equal to 0.1 pM. In some aspects, an anti-SLO antibody binds to an epitope of SLO that is conserved among SLO proteins from different species (e.g., S. pyogenes and S. dysgalactiae SLO). An antibody binds specifically to a SLO antigen when it binds to a SLO antigen with higher affinity than to any cross-reactive antigen as determined using experimental techniques, such as radioimmunoassays (RIA) and enzyme linked immunosorbent assays (ELISAs). Typically a specific or selective reaction will be at least twice background signal or noise and can be more than 10 times background. See, e.g., Paul, ed., 1989, Fundamental Immunology Second Edition, Raven Press, New York at pages 332-336 for a discussion regarding antibody specificity. In some aspects, the extent of binding of the antibody to a “non-target” protein will be less than about 10% of the binding of the antibody to its particular target protein, for example, as determined by fluorescence activated cell sorting (FACS) analysis or radioimmunoprecipitation (RIA).

As used herein, in reference to an antibody, the term “heavy (H) chain” refers to a polypeptide chain of about 50-70 kDa, wherein the amino-terminal portion includes a variable (V) region (also called V domain) of about 115 to 130 or more amino acids and a carboxy-terminal portion that includes a constant (C) region. The constant region (or constant domain) can be one of five distinct types, (e.g., isotypes) referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while p and E contain approximately 550 amino acids. When combined with a light chain, these distinct types of heavy chains give rise to five well known classes (e.g., isotypes) of antibodies, namely, IgA, IgD, IgE, IgG and IgM, respectively, including four subclasses of IgG, namely IgG1, IgG2, IgG3 and IgG4. An antibody heavy chain can be a human antibody heavy chain.

As used herein in reference to an antibody, the term “light (L) chain” refers to a polypeptide chain of about 25 kDa, wherein the amino-terminal portion includes a variable domain of about 100 to about 110 or more amino acids and a carboxy-terminal portion that includes a constant region. The approximate length of a light chain (both the V and C domains) is 211 to 217 amino acids. There are two distinct types of light chains, referred to as kappa (κ) and lambda (λ), based on the amino acid sequence of the constant domains. Light chain amino acid sequences are well known in the art. An antibody light chain can be a human antibody light chain.

As used herein, the term “variable (V) region” or “variable (V) domain” refers to a portion of the light (L) or heavy (H) chains of an antibody polypeptide that is generally located at the amino-terminus of the L or H chain. The H chain V domain has a length of about 115 to 130 amino acids, while the L chain V domain is about 100 to 110 amino acids in length. The H and L chain V domains are used in the binding and specificity of each particular antibody for its particular antigen. The V domain of the H chain can be referred to as “VH.” The V region of the L chain can be referred to as “VL.” The term “variable” refers to the fact that certain segments of the V domains differ extensively in sequence among different antibodies. While the V domain mediates antigen binding and defines specificity of a particular antibody for its particular antigen, the variability is not evenly distributed across the 110-amino acid span of antibody V domains. Instead, the V domains consist of less variable (e.g., relatively invariant) stretches called framework regions (FRs) of about 15-30 amino acids separated by shorter regions of greater variability (e.g., extreme variability) called “hypervariable regions” or “complementarity determining regions” (CDRs) that are each about 9-12 amino acids long or 3-17 amino acids long. The V domains of antibody H and L chains each comprise four FRs, largely adopting a β sheet configuration, connected by three hypervariable regions, called, which form loops connecting, and in some cases forming part of, the β sheet structure. The hypervariable regions in each chain are held together in close proximity by the FRs and, with the hypervariable regions from the other chain, contribute to the formation of the antigen-binding site of antibodies (see, e.g., Kabat et al., 1991, Sequences of Proteins of Immunological Interest, 5th Ed. Public Health Service, National Institutes of Health, Bethesda, MD). The C domains are not involved directly in binding an antibody to an antigen, but exhibit various effector functions, such as antibody dependent cellular cytotoxicity (ADCC) and complement dependent cytotoxicity (CDC). The V domains differ extensively in sequence among different antibody classes or types. The variability in sequence is concentrated in the CDRs, which are primarily responsible for the interaction of the antibody with antigen. In some aspects, the variable domain of an antibody is a human or humanized variable domain.

As used herein, the terms “complementarity determining region,” “CDR,” “hypervariable region,” “HVR,” and “HV” are used interchangeably. A “CDR” refers to one of three hypervariable regions (H1, H2 or H3) within the non-framework region of the antibody VH β-sheet framework, or to one of three hypervariable regions (L1, L2 or L3) within the non-framework region of the antibody VL β-sheet framework. The term, when used herein, refers to the regions of an antibody V domain that are hypervariable in sequence and/or form structurally defined loops. Generally, antibodies comprise six hypervariable regions: three (H1, H2, H3) in the VH domain and three (L1, L2, L3) in the VL domain.

Accordingly, CDRs are typically highly variable sequences interspersed within the framework region sequences of the V domain. “Framework” or “FR” residues are those variable region residues flanking the CDRs. FR residues are present, for example, in chimeric, humanized, human, domain antibodies, diabodies, linear antibodies, and bispecific antibodies.

A number of hypervariable region delineations are in use and are encompassed herein. CDR regions are well known to those skilled in the art and have been defined by, for example, Kabat as the regions of most hypervariability within the antibody V domains (Kabat et al., 1977, J. Biol. Chem., 252:6609-6616; Kabat, 1978, Adv. Prot. Chem., 32:1-75). The Kabat CDRs are based on sequence variability and are the most commonly used (see, e.g., Kabat et al., 1991, Sequences of Proteins of Immunological Interest, 5th Ed. Public Health Service, National Institutes of Health, Bethesda, MD). CDR region sequences also have been defined structurally by Chothia as those residues that are not part of the conserved β-sheet framework, and thus are able to adopt different conformations (Chothia et al., 1987, J. Mol. Biol., 196:901-917). Chothia refers instead to the location of the structural loops. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). Both numbering systems and terminologies are well recognized in the art.

Recently, a universal numbering system has been developed and widely adopted, ImMunoGeneTics (IMGT) Information System® (Lafranc et al., 2003, Dev. Comp. Immunol., 27(1):55-77). IMGT is an integrated information system specializing in immunoglobulins (Ig), T cell receptors (TR) and the major histocompatibility complex (MHC) of human and other vertebrates. Herein, the CDRs are referred to in terms of both the amino acid sequence and the location within the light or heavy chain. As the “location” of the CDRs within the structure of the immunoglobulin V domain is conserved between species and present in structures called loops, by using numbering systems that align variable domain sequences according to structural features, CDR and framework residues and are readily identified. This information can be used in grafting and in the replacement of CDR residues from immunoglobulins of one species into an acceptor framework from, typically, a human antibody. An additional numbering system (AHon) has been developed by Honegger et al., 2001, J. Mol. Biol., 309: 657-670. Correspondence between the numbering system, including, for example, the Kabat numbering and the IMGT unique numbering system, is well known to one skilled in the art (see, e.g., Kabat, Id; Chothia et al., Id.; Martin, 2010, Antibody Engineering, Vol. 2, Chapter 3, Springer Verlag; and Lefranc et al., 1999, Nuc. Acids Res., 27:209-212).

CDR region sequences have also been defined by AbM, Contact and IMGT. The AbM hypervariable regions represent a compromise between the Kabat CDRs and Chothia structural loops, and are used by Oxford Molecular's AbM antibody modeling software (see, e.g., Martin, 2010, Antibody Engineering, Vol. 2, Chapter 3, Springer Verlag). The “contact” hypervariable regions are based on an analysis of the available complex crystal structures. The residues from each of these hypervariable regions or CDRs are noted below.

Exemplary delineations of CDR region sequences are illustrated in Table 2. The positions of CDRs within a canonical antibody variable region have been determined by comparison of numerous structures (Al-Lazikani et al., 1997, J. Mol. Biol., 273:927-948); Morea et al., 2000, Methods, 20:267-279). Because the number of residues within a hypervariable region varies in different antibodies, additional residues relative to the canonical positions are conventionally numbered with a, b, c and so forth next to the residue number in the canonical variable region numbering scheme (Al-Lazikani et al., Id). Such nomenclature is similarly well known to those skilled in the art.

An “affinity matured” antibody is one with one or more alterations (e.g., amino acid sequence variations, including changes, additions and/or deletions) in one or more HVRs thereof that result in an improvement in the affinity of the antibody for antigen, compared to a parent antibody which does not possess those alteration(s). In some aspects, affinity matured antibodies will have nanomolar or even picomolar affinities for the target antigen, such as SLO. Affinity matured antibodies are produced by procedures known in the art. For reviews, see Hudson and Souriau, 2003, Nature Medicine, 9:129-134; Hoogenboom, 2005, Nature Biotechnol., 23:1105-1116; Quiroz and Sinclair, 2010, Revista Ingeneria Biomedia, 4: 39-51.

A “chimeric” antibody is one in which a portion of the H and/or L chain, e.g., the V domain, is identical with or homologous to a corresponding amino acid sequence in an antibody derived from a particular species or belonging to a particular antibody class or subclass, while the remainder of the chain(s), e.g., the C domain, is identical with or homologous to corresponding sequences in antibodies derived from another species or belonging to another antibody class or subclass, as well as a fragment of such an antibody, so long as it exhibits the desired biological activity (see, e.g., U.S. Pat. No. 4,816,567; and Morrison et al., 1984, Proc. Natl. Acad. Sci. USA, 81:6851-6855).

The term “humanized antibody” refers to forms of antibodies that contain sequences from non-human (e.g., murine) antibodies as well as human antibodies. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. Furthermore, humanized antibodies can comprise residues that are found neither in the recipient antibody nor in the imported CDR or framework sequences. These modifications are made to further refine and maximize antibody performance. Thus, in general, a humanized antibody can comprise all or substantially all of at least one, and in one aspect two, variable domains, in which all or substantially all of the hypervariable loops correspond to those of a non-human immunoglobulin and all or substantially all of the FR regions are those of a human immunoglobulin sequence. The humanized antibody optionally also can comprise at least a portion of an immunoglobulin constant region (Fc), or that of a human immunoglobulin (see, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1; Queen et al. (1989) Proc. Natl. Acad. Sci. USA, Vol 86:10029-10033). The terms “human antibody” and “fully human antibody” are used interchangeably herein and refer to an antibody that possesses an amino acid sequence which corresponds to that of an antibody produced by a human and/or has been made using any of the techniques for making human antibodies as practiced by those skilled in the art. This definition of a human antibody specifically excludes a humanized antibody comprising non-human antigen-binding residues. Human antibodies can be produced using various techniques known in the art, including phage-display libraries (Hoogenboom et al., 1991, J. Mol. Biol., 227:381; Marks et al., 1991, J. Mol. Biol., 222:581 and yeast display libraries (Chao et al., 2006, Nature Protocols, 1:755-768). Also available for the preparation of human monoclonal antibodies are methods described in Cole et al., 1985 Monoclonal Antibodies and Cancer Therapy, Alan R. Liss, p. 77; Boemer et al., 1991, J. Immunol., 147(1):86-95. See also van Dijk et al., 2001, Curr. Opin. Pharmacol., 5: 368-74. Human antibodies can be prepared by administering an antigen to a transgenic animal whose endogenous Ig loci have been disabled, e.g., a mouse, and that has been genetically modified to harbor human immunoglobulin genes which encode human antibodies, such that human antibodies are generated in response to antigenic challenge (see, e.g., Jakobovits, A., 1995, Curr. Opin. Biotechnol. 6(5):561-566; Bruggemann et al., 1997 Curr. Opin. Biotechnol., 8(4):455-8; and U.S. Pat. Nos. 6,075,181 and 6,150,584 regarding XENOMOUSE™ technology). See also, for example, Li et al., 2006, Proc. Natl. Acad. Sci. USA, 103:3557-3562 regarding human antibodies generated via a human B-cell hybridoma technology. In some aspects, human antibodies comprise a variable region and constant region of human origin. “Fully human” anti-SLO antibodies, in some aspects, can also encompass antibodies which bind SLO polypeptides and are encoded by nucleic acid sequences which are naturally occurring somatic variants of human germline immunoglobulin nucleic acid sequence. In some aspects, the anti-SLO antibodies provided herein are fully human antibodies. The term “fully human antibody” includes antibodies having variable and constant regions corresponding to human germline immunoglobulin sequences as described by Kabat et al. (See Kabat et al., 1991, Sequences of Proteins of Immunological Interest, Fifth Edition, U.S. Department of Health and Human Services, NIH Publication No. 91-3242). The phrase “recombinant human antibody” includes human antibodies that are prepared, expressed, created, or isolated by recombinant means, such as antibodies expressed using a recombinant expression vector transfected into a host cell; antibodies isolated from a recombinant, combinatorial human antibody library; antibodies isolated from an animal (e.g., a mouse or cow) that is transgenic and/or transchromosomal for human immunoglobulin genes (see e.g., Taylor, L. D. et al., 1992, Nucl. Acids Res. 20:6287-6295); or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat et al., 1991, Sequences of Proteins of Immunological Interest, Fifth Edition, U.S. Department of Health and Human Services, NIH Publication No. 91-3242). In some aspects, however, such recombinant human antibodies are subjected to in vitro mutagenesis (or, when an animal transgenic for human Ig sequences is used, in vivo somatic mutagenesis) and, thus, the amino acid sequences of the VH and VL regions of the recombinant antibodies are sequences that, while derived from and related to human germline VH and VL sequences, may not naturally exist within the human antibody germline repertoire in vivo.

As used herein, the term “epitope” is the site(s) or region(s) on the surface of an antigen molecule to which a single antibody molecule binds, such as a localized region on the surface of an antigen, e.g., a SLO polypeptide that is capable of being bound by one or more antigen binding regions of an anti-SLO antibody. An epitope can be immunogenic and capable of eliciting an immune response in an animal. Epitopes need not necessarily be immunogenic. Epitopes often consist of chemically active surface groupings of molecules such as amino acids or sugar side chains and have specific three dimensional structural characteristics as well as specific charge characteristics. An epitope can be a linear epitope and a conformational epitope. A region of a polypeptide contributing to an epitope can be contiguous amino acids of the polypeptide, forming a linear epitope, or the epitope can be formed from two or more non-contiguous amino acids or regions of the polypeptide, typically called a conformational epitope. The epitope may or may not be a three-dimensional surface feature of the antigen. In some aspects, a SLO epitope is a three-dimensional surface feature of a SLO polypeptide. In some aspects, a SLO epitope is linear feature of a SLO polypeptide.

An antibody binds “an epitope” or “essentially the same epitope” or “the same epitope” as a reference antibody, when the two antibodies recognize identical, overlapping, or adjacent epitopes in a three-dimensional space. The most widely used and rapid methods for determining whether two antibodies bind to identical, overlapping, or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies to antigen is measured using a detectable signal, e.g., radioactive, fluorescent or enzyme labels.

The term “compete” when used in the context of anti-SLO antibodies that compete for the same epitope or binding site on a SLO target protein or peptide thereof means competition as determined by an assay in which the antibody under study, or binding fragment thereof, prevents, blocks, or inhibits the specific binding of a reference molecule (e.g., a reference ligand, or reference antigen binding protein, such as a reference antibody) to a common antigen (e.g., SLO or a fragment thereof). Numerous types of competitive binding assays can be used to determine if a test antibody competes with a reference antibody for binding to SLO. Examples of assays that can be employed include solid phase direct or indirect radioimmunoassay (RIA); solid phase direct or indirect enzyme immunoassay (EIA); sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619); solid phase direct labeled assay; solid phase direct labeled sandwich assay (see, e.g., Harlow and Lane, 1988, Antibodies, A Laboratory Manual, Cold Spring Harbor Press); solid phase direct label RIA using labeled iodine (1125 label) (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of a purified antigen (e.g., SLO) bound to a solid surface, or cells bearing either of an unlabeled test antigen binding protein (e.g., test anti-SLO antibody) or a labeled reference antigen binding protein (e.g., reference anti-SLO antibody). Competitive inhibition can be measured by determining the amount of label bound to the solid surface or cells in the presence of a known amount of the test antigen binding protein. Usually the test antigen binding protein is present in excess. Antibodies identified by competition assay (competing antibodies) include antibodies binding to the same epitope as the reference antibody and/or antibodies binding to an adjacent epitope sufficiently proximal to the epitope bound by the reference antibody causing steric hindrance to occur. Additional details regarding methods for determining competitive binding are described herein. Usually, when a competing antibody protein is present in excess, it will inhibit specific binding of a reference antibody to a common antigen by at least 15%, or at least 20%, for example, without limitation, 25%, 30%, 35%, 40%, 45%, 50%, 55%, 60%, 65%, 70% or 75% or greater, as well as percent amounts between the amounts stated. In some aspects, binding can be inhibited by at least 80%, 85%, 90%, 95%, 96% or 97%, 98%, 99% or more.

As used herein, the term “blocking” antibody or an “antagonist” antibody refers to an antibody that prevents, inhibits, blocks, or reduces biological or functional activity of the antigen to which it binds. Blocking antibodies or antagonist antibodies can substantially or completely prevent, inhibit, block, or reduce the biological activity or function of the antigen. For example, a blocking anti-SLO antibody can prevent, inhibit, block, or reduce the binding interaction between SLO and a cholesterol-containing host cell membrane or a carbohydrate-containing host cell membrane, thus preventing, blocking, inhibiting, or reducing SLO-induced red blood cell hemolysis or epithelial cell cytotoxicity. The terms block, inhibit, and neutralize are used interchangeably herein and refer to the ability of the anti-SLO antibodies to prevent or otherwise disrupt or reduce the SLO/host cell membrane interaction.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, certain changes and modifications may be practiced within the scope of the appended claims.

### Compositions

Anti-SLO antibodies. Disclosed herein are anti-SLO antibodies or binding fragments thereof. Disclosed herein are anti-SLO antibodies or binding fragments thereof that bind to SLO. Disclosed herein are anti-SLO antibodies or binding fragments thereof that bind to SLO and block or inhibit binding of streptolysin O to a cholesterol-containing host cell membrane or a carbohydrate-containing host cell membrane. Disclosed herein are anti-SLO antibodies or binding fragments thereof useful in treating necrotizing soft tissue infections. Also disclosed herein are anti-SLO antibodies or binding fragments thereof useful in the inhibiting or preventing streptolysin O-induced RBC-hemolysis, and preventing or inhibiting cytotoxicity in epithelial cells. Further disclosed herein are anti-SLO antibodies or binding fragments thereof useful in treating skin or dermal infections or strep throat. In some aspects, the anti-SLO antibodies or binding fragments thereof described herein can be used to diagnose an infection caused by a Streptococcus species. For example, detecting the presence of one or more SLO antibodies in sample obtained from a subject can indicate an infection (e.g., strep throat) caused by a Streptococcus species.

The anti-SLO antibodies may be of the IgG, IgM, IgA, IgD, and IgE Ig classes, as well as polypeptides comprising one or more antibody CDR domains that retain antigen binding activity. Illustratively, the anti-SLO antibodies may be chimeric, affinity matured, humanized, or human antibodies. In some aspects, the anti-SLO antibodies can be monoclonal antibodies. In some aspects, the monoclonal anti-SLO antibody can be a humanized antibody. By known means and as described herein, polyclonal or monoclonal antibodies, antibody fragments, binding domains and CDRs (including engineered forms of any of the foregoing) may be created that are specific for SLO antigen, one or more of its respective epitopes, or conjugates of any of the foregoing, whether such antigens or epitopes are isolated from natural sources or are synthetic derivatives or variants of the natural protein.

Also disclosed herein are compositions comprising anti-SLO antibodies. In some aspects, the antibodies disclosed herein can be isolated antibodies. Examples of the CDR sequences and heavy or light chain variable region sequences of anti-SLO antibodies are shown in Table 3.

Disclosed herein are isolated antibodies comprising a light chain variable region and a heavy chain variable region. In some aspects, the light chain variable region can comprise a complementarity determining region light chain 1 (CDRL1) amino acid sequence of SEQ ID NO: 1, or SEQ ID NO: 1 having at least 1 conservative substitution; a complementarity determining region light chain 2 (CDRL2) amino acid sequence of SEQ ID NO: 2, or SEQ ID NO: 2 having at least 1 conservative substitution; and a complementarity determining region light chain 3 (CDRL3) amino acid sequence of SEQ ID NO: 3, or SEQ ID NO: 3 having at least 1 conservative substitution. In some aspects, the heavy chain variable region can comprise a complementarity determining region heavy chain 1 (CDRH1) amino acid sequence of SEQ ID NO: 8, or SEQ ID NO: 8 having at least 1 conservative substitution; a complementarity determining region heavy chain 2 (CDRH2) amino acid sequence of SEQ ID NO: 9, or SEQ ID NO: 9 having at least 1 conservative substitution; and a complementarity determining region heavy chain 3 (CDRH3) amino acid sequence of SEQ ID NO: 10, or SEQ ID NO: 10 having at least 1 conservative substitution.

In some aspects, the light chain variable region can comprise the CDRL1 amino acid sequence of SEQ ID NO: 1; the CDRL2 amino acid sequence of SEQ ID NO: 2; and the CDRL3 amino acid sequence of SEQ ID NO: 3; and the heavy chain variable region can comprise the CDRH1 amino acid sequence of SEQ ID NO: 4; the CDRH2 amino acid sequence of SEQ ID NO: 5; and the CDRH3 amino acid sequence of SEQ ID NO: 6.

Also disclosed herein, are isolated antibodies comprising a light chain variable region amino acid sequence of SEQ ID NO: 7 and a heavy chain variable region amino acid sequence of SEQ ID NO: 8.

In some aspects, any of the antibodies disclosed herein can comprise a light chain variable region amino acid sequence comprising SEQ ID NO: 7. In some aspects, any of the antibodies disclosed herein can comprise a heavy chain variable region amino acid sequence comprising SEQ ID NO: 8. In some aspects, any of the antibodies disclosed herein can comprise a light chain variable region amino acid sequence comprising an amino acid sequence that is at least 90% identical to amino acid sequence SEQ ID NO: 7. In some aspects, any of the antibodies disclosed herein can comprise a heavy chain variable region amino acid sequence comprising an amino acid sequence that is at least 90% identical to amino acid sequence SEQ ID NO: 8.

Disclosed herein are isolated antibodies comprising a light chain variable region and a heavy chain variable region. In some aspects, the light chain variable region can comprise a complementarity determining region light chain 1 (CDRL1) amino acid sequence of SEQ ID NO: 9, or SEQ ID NO: 9 having at least 1 conservative substitution; a complementarity determining region light chain 2 (CDRL2) amino acid sequence of SEQ ID NO: 10, or SEQ ID NO: 10 having at least 1 conservative substitution; and a complementarity determining region light chain 3 (CDRL3) amino acid sequence of SEQ ID NO: 11, or SEQ ID NO: 11 having at least 1 conservative substitution. In some aspects, the heavy chain variable region can comprise a complementarity determining region heavy chain 1 (CDRH1) amino acid sequence of SEQ ID NO: 12, or SEQ ID NO: 12 having at least 1 conservative substitution; a complementarity determining region heavy chain 2 (CDRH2) amino acid sequence of SEQ ID NO: 13, or SEQ ID NO: 13 having at least 1 conservative substitution; and a complementarity determining region heavy chain 3 (CDRH3) amino acid sequence of SEQ ID NO: 14, or SEQ ID NO: 14 having at least 1 conservative substitution.

In some aspects, the light chain variable region can comprise the CDRL1 amino acid sequence of SEQ ID NO: 9; the CDRL2 amino acid sequence of SEQ ID NO: 10; and the CDRL3 amino acid sequence of SEQ ID NO: 11; and the heavy chain variable region can comprise the CDRH1 amino acid sequence of SEQ ID NO: 12; the CDRH2 amino acid sequence of SEQ ID NO: 13; and the CDRH3 amino acid sequence of SEQ ID NO: 14.

Also disclosed herein, are isolated antibodies comprising a light chain variable region amino acid sequence of SEQ ID NO: 15 and a heavy chain variable region amino acid sequence of SEQ ID NO: 16.

In some aspects, any of the antibodies disclosed herein can comprise a light chain variable region amino acid sequence comprising SEQ ID NO: 15. In some aspects, any of the antibodies disclosed herein can comprise a heavy chain variable region amino acid sequence comprising SEQ ID NO: 16. In some aspects, any of the antibodies disclosed herein can comprise a light chain variable region amino acid sequence comprising an amino acid sequence that is at least 90% identical to amino acid sequence SEQ ID NO: 15. In some aspects, any of the antibodies disclosed herein can comprise a heavy chain variable region amino acid sequence comprising amino acid sequence that is at least 90% identical to amino acid sequence SEQ ID NO: 16.

Disclosed herein are isolated antibodies comprising a light chain variable region and a heavy chain variable region. In some aspects, the light chain variable region can comprise a complementarity determining region light chain 1 (CDRL1) amino acid sequence of SEQ ID NO: 17, or SEQ ID NO: 17 having at least 1 conservative substitution; a complementarity determining region light chain 2 (CDRL2) amino acid sequence of SEQ ID NO: 2, or SEQ ID NO: 2 having at least 1 conservative substitution; and a complementarity determining region light chain 3 (CDRL3) amino acid sequence of SEQ ID NO: 18, or SEQ ID NO: 18 having at least 1 conservative substitution. In some aspects, the heavy chain variable region can comprise a complementarity determining region heavy chain 1 (CDRH1) amino acid sequence of SEQ ID NO: 19, or SEQ ID NO: 19 having at least 1 conservative substitution; a complementarity determining region heavy chain 2 (CDRH2) amino acid sequence of SEQ ID NO: 20, or SEQ ID NO: 20 having at least 1 conservative substitution; and a complementarity determining region heavy chain 3 (CDRH3) amino acid sequence of SEQ ID NO: 21, or SEQ ID NO: 21 having at least 1 conservative substitution.

In some aspects, the light chain variable region can comprise the CDRL1 amino acid sequence of SEQ ID NO: 17; the CDRL2 amino acid sequence of SEQ ID NO: 2; and the CDRL3 amino acid sequence of SEQ ID NO: 18; and the heavy chain variable region can comprise the CDRH1 amino acid sequence of SEQ ID NO: 19; the CDRH2 amino acid sequence of SEQ ID NO: 20; and the CDRH3 amino acid sequence of SEQ ID NO: 21.

Also disclosed herein, are isolated antibodies comprising a light chain variable region amino acid sequence of SEQ ID NO: 22 and a heavy chain variable region amino acid sequence of SEQ ID NO: 23.

In some aspects, any of the antibodies disclosed herein can comprise a light chain variable region amino acid sequence comprising SEQ ID NO: 22. In some aspects, any of the antibodies disclosed herein can comprise a heavy chain variable region amino acid sequence comprising SEQ ID NO: 23. In some aspects, any of the antibodies disclosed herein can comprise a light chain variable region amino acid sequence comprising an amino acid sequence that is at least 90% identical to amino acid sequence SEQ ID NO: 22. In some aspects, any of the antibodies disclosed herein can comprise a heavy chain variable region amino acid sequence comprising an amino acid sequence that is at least 90% identical to amino acid sequence SEQ ID NO: 23.

TABLE 3
Exemplary Amino Acid Sequences of Anti-SLO Antibodies

| SEQ ID NO: | Sequence | Description |
|---|---|---|
| 1 | NIGTKN | CDRL1, 55c11 IgG |
| | | antibody |
| 2 | DDS | CDRL2, 55c11 IgG |
| | | antibody; and CDRL2, |
| | | 55G4 IgG antibody |
| 3 | QVWDSDNDRWL | CDRL3, 55c11 IgG |
| | | antibody |
| 4 | GFTFSS | CDRH1, 55c11 IgG |
| | | antibody |
| 5 | INTISTFI | CDRH2, 55c11 IgG |
| | | antibody |
| 6 | ARERGNRFCDGDCYSGTTFDY | CDRH3, 55c11 IgG |
| | | antibody |
| 7 | QPVLTQPPSVSVAPGRTSRITCE | Lambda Light Chain |
| | ENNIGTKNVHWYQQKPGQAPL | Variable Domain, 55c11 |
| | LVVHDDSDRPSGIPERFSGSNSG | IgG antibody |
| | NTATLTISRVEAGDEADYYCQVW | |
| | DSDNDRWLFGGGTRLTVLGQPKAA | |
| 8 | QVQLVESGGGLVKPGGSLRLSCAASG | Heavy Chain Variable |
| | FTFSSHSMNWVRQTPGKGLEWVSSIN | Domain, 55c11 IgG |
| | TISTFIYYADSVKGRFTVSRDNARNSL | antibody |
| | FLQMNTLTADDTGVYYCARERGNRF | |
| | CDGDCYSGTTFDYWGPGTLVTVSSA | |
| 9 | QDISNY | CDRL1, 55L17 IgG |
| | | antibody |
| 10 | KAS | CDRL2, 55L17 IgG |
| | | antibody |
| 11 | QQYEEYLRYS | CDRL3, 55L17 IgG |
| | | antibody |
| 12 | GGTLSNYA | CDRH1, 55L17 IgG |
| | | antibody |
| 13 | IVPMFSTT | CDRH2, 55L17 IgG |
| | | antibody |
| 14 | ARDLPAPYCSADLCYKVLDAFDV | CDRH3, 55L17 IgG |
| | | antibody |
| 15 | EIVLTQSPSSLSASVGDRVTITCQA | Kappa Light Chain |
| | SQDISNYLNWYQQKPGKAPKLLIY | Variable Domain, 55L17 |
| | KASSLESGVPSRFSGSGSGTEFTLTI | IgG antibody |
| | TRLQPDDFATYYCQQYEEYLRYSF | |
| | GQGTKLEIK | |
| 16 | QVQLVQSGAEVKKPGSSVKVSCRP | Heavy Chain Variable |
| | PGGTLSNYAINWVRQAPGQGLEW | Domain, 55L17 IgG |
| | MGGIVPMFSTTKYAQKFQGRLTLT | antibody |
| | ADLSTITAHMELNRLTSEDTAVYY | |
| | CARDLPAPYCSADLCYKVLDAFDV | |
| | WGQGTLVTVSS | |
| 17 | NIGRKH | CDRL1, 55G4 IgG |
| | | antibody |
| 18 | QVWDNDAEQWV | CDRL3, 55LG4 IgG |
| | | antibody |
| 19 | GFTFSNFP | CDRH1, 55G4 IgG |
| | | antibody |
| 20 | ISHDETMI | CDRH2, 55G4 IgG |
| | | antibody |
| 21 | ARDTGVCSGGYCYTPRYFDQ | CDRH3, 55G4 IgG |
| | | antibody |
| 22 | QPVLTQPPSVSVAPGKTADITCGGD | Lambda Light Chain |
| | NIGRKHVHWYRQRPSQAPLLVIYD | Variable Domain, 55G4 |
| | DSDRPSGIPERLSGINSGNTATLTIS | IgG antibody |
| | GVEVGDEADYYCQVWDNDAEQW | |
| | VFGGGTKLTV | |
| 23 | EVQLVQSGGGAVQPGRSLRLSCAVS | Heavy Chain Variable |
| | GFTFSNFPMHWVRQAPGRGLEWVA | Domain, 55G4 IgG |
| | VISHDETMIYYLDSVKGRFTISRDNSR | antibody |
| | NIVYLQMHSLRVEDTAVYYCARDTG | |
| | VCSGGYCYTPRYFDQWGQGTLVTVSS | |

In some aspects, any of the antibodies disclosed herein can comprise variants of the CDR sequences disclosed herein. Generally, the amino acid identity between individual variant CDRs is at least 90%, 91%, 92%, 93%, 94%, 95%, 96%, 97%, 98%, 99%, or 100%. Thus, a “variant CDR” is one with the specified identity to the parent or reference CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. For example, a “variant CDR” can be a sequence that contains 1, 2, 3, 4 or 5 amino acid changes as compared to the parent or reference CDR of the invention, and shares or improves biological function, specificity and/or activity of the parent CDR.

In some aspects, any of CDR sequences disclosed herein can include a single amino acid change as compared to the parent or reference CDR. In some aspects, any of the CDR sequences disclosed herein can include at least two amino acid changes as compared to the parent or reference CDR. In some aspects, the amino acid change can be a change from a cysteine residue to another amino acid. In some aspects, the amino acid change can be a change from a glycine residue to another amino acid. The amino acid identity between individual variant CDRs can be at least 60%, 65%, 70%, 75%, 80%, 85%, 90%, 91%, 92%, 93%, 94%, 95%, 96%, 97%, 98%, 99% or 100%. Thus, a “variant CDR” can be one with the specified identity to the parent CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. For example, the parent CDR sequence can be one or more of SEQ ID NOs: 1, 2, 3, 4, 5, 6, 8, 9, 10, 11, 12, and/or 13. The variant CDR sequence can be at least 60%, 65%, 70%, 75%, 80%, 85%, 90%, 91%, 92%, 93%, 94%, 95%, 96%, 97%, 98%, 99% or 100% identical to any one of SEQ ID NOs: 1, 2, 3, 4, 5, 6, 8, 9, 10, 11, 12, and/or 13. The variant CDR sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

As discussed herein, minor variations in the amino acid sequences of any of the antibodies disclosed herein are contemplated as being encompassed by the instant disclosure, providing that the variations in the amino acid sequence maintains at least 75%, more preferably at least 80%, 90%, 95%, and most preferably 99% sequence identity to the parent sequence. In some aspects, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are known to one of ordinary skill in the art.

In some aspects, amino acid substitutions can be those which: (1) reduce susceptibility to proteolysis, (2) reduce susceptibility to oxidation, (3) alter binding affinity for forming protein complexes, (4) alter binding affinities, and (4) confer or modify other physiochemical or functional properties of such analogs. In some aspects, single or multiple amino acid substitutions (preferably conservative amino acid substitutions) may be made in the non-CDR sequence of the heavy chain, the light chain or both. In some aspects, one or more amino acid substitutions can be made in one or more of the CDR sequences of the heavy chain, the light chain or both.

Many methods have been developed for chemical labeling and enhancement of the properties of antibodies and their common fragments, including the Fab and F(ab′)2 fragments. Somewhat selective reduction of some antibody disulfide bonds has been previously achieved, yielding antibodies and antibody fragments that can be labeled at defined sites, enhancing their utility and properties. Selective reduction of the two hinge disulfide bonds present in F(ab′)2 fragments using mild reduction has been useful. In some aspects, cysteine and methionine can be susceptible to rapid oxidation, which can negatively influence the cleavage of protecting groups during synthesis and the subsequent peptide purification. In some instances, cysteine residues in peptides used for antibody production can affect the avidity of the antibody, because free cysteines are uncommon in vivo and therefore may not be recognized by the native peptide structure. In some aspects, the disclosed antibodies and fragments thereof comprise a sequence where a cysteine reside outside of the CDR (e.g. in the non-CDR sequence of the heavy chain, the light chain or both) is substituted. In some aspects, cysteine can be replaced with serine and methionine replaced with norleucine (Nle). Multiple cysteines on a peptide or in one of the disclosed antibodies or fragments thereof may be susceptible to forming disulfide linkages unless a reducing agent such as dithiothreitol (DTT) is added to the buffer or the cysteines can be replaced with serine residues.

While the site or region for introducing an amino acid sequence variation is predetermined, the mutation per se need not be predetermined. For example, in order to optimize the performance of a mutation at a given site, random mutagenesis may be conducted at the target codon or region and the expressed antigen binding protein CDR variants screened for the optimal combination of desired activity. Techniques for making substitution mutations at predetermined sites in DNA having a known sequence are well known, for example, M13 primer mutagenesis and PCR mutagenesis. Screening of the mutants is done using assays of antigen binding protein activities as described herein.

Amino acid substitutions are typically of single residues; insertions usually will be on the order of from about one (1) to about twenty (20) amino acid residues, although considerably larger insertions may be tolerated. Deletions range from about one (1) to about twenty (20) amino acid residues, although in some cases deletions may be much larger.

Substitutions, deletions, insertions or any combination thereof may be used to arrive at a final derivative or variant. Generally these changes are done on a few amino acids to minimize the alteration of the molecule, particularly the immunogenicity and specificity of the antigen binding protein. However, larger changes may be tolerated in certain circumstances.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein, or any other antibody embodiments as outlined herein.

By “Fv” or “Fv fragment” or “Fv region” as used herein is meant a polypeptide that comprises the VL and VH domains of a single antibody.

By “framework” as used herein is meant the region of an antibody variable domain exclusive of those regions defined as CDRs. Each antibody variable domain framework can be further subdivided into the contiguous regions separated by the CDRs (FR1, FR2, FR3 and FR4).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are as described in Table 1, supra. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Disclosed herein are isolated antibodies or antibody fragments thereof comprising a light chain variable region and a heavy chain variable region, wherein the light chain variable region comprises one or more variant CDRs. In some aspects, the variant complementarity determining region light chain 1 (CDRL1) comprises one or two conservative amino acid substitutions at positions 26-31 of SEQ ID NO: 7. In some aspects, the variant complementarity determining region light chain 2 (CDRL2) comprises one or two conservative amino acid substitutions at positions 49-51 of SEQ ID NO: 7. In some aspects, the variant complementarity determining region light chain 3 (CDRL3) comprises one or two conservative amino acid substitutions at positions 88-98 of SEQ ID NO: 7. Also disclosed herein are isolated antibodies or antibody fragments thereof comprising a light chain variable region and a heavy chain variable region, wherein the heavy chain variable region comprises variant complementarity determining regions. In some aspects, the variant complementarity determining region heavy chain 1 (CDRH1) comprises one or two conservative amino acid substitutions at positions 26-31 of SEQ ID NO: 8. In some aspects, the variant complementarity determining region heavy chain 2 (CDRH2) comprising one or two conservative amino acid substitutions at positions 51-58 of SEQ ID NO: 8. In some aspects, the variant complementarity determining region heavy chain 3 (CDRH3) comprises one or two conservative amino acid substitutions at positions 97-117 of SEQ ID NO: 8.

In some aspects, the variant complementarity determining region light chain 1 (CDRL1) comprises one or two conservative amino acid substitutions at positions 27-32 of SEQ ID NO: 15. In some aspects, the variant complementarity determining region light chain 2 (CDRL2) comprises one or two conservative amino acid substitutions at positions 50-52 of SEQ ID NO: 15. In some aspects, the variant complementarity determining region light chain 3 (CDRL3) comprises one or two conservative amino acid substitutions at positions 89-98 of SEQ ID NO: 15. In some aspects, the variant complementarity determining region heavy chain 1 (CDRH1) comprises one or two conservative amino acid substitutions at positions 26-33 of SEQ ID NO: 16. In some aspects, the variant complementarity determining region heavy chain 2 (CDRH2) comprising one or two conservative amino acid substitutions at positions 51-58 of SEQ ID NO: 16. In some aspects, the variant complementarity determining region heavy chain 3 (CDRH3) comprises one or two conservative amino acid substitutions at positions 97-119 of SEQ ID NO: 16.

In some aspects, the variant complementarity determining region light chain 1 (CDRL1) comprises one or two conservative amino acid substitutions at positions 26-31 of SEQ ID NO: 22. In some aspects, the variant complementarity determining region light chain 2 (CDRL2) comprises one or two conservative amino acid substitutions at positions 49-51 of SEQ ID NO: 22. In some aspects, the variant complementarity determining region light chain 3 (CDRL3) comprises one or two conservative amino acid substitutions at positions 88-98 of SEQ ID NO: 22. In some aspects, the variant complementarity determining region heavy chain 1 (CDRH1) comprises one or two conservative amino acid substitutions at positions 26-33 of SEQ ID NO: 23. In some aspects, the variant complementarity determining region heavy chain 2 (CDRH2) comprises one or two conservative amino acid substitutions at positions 51-58 of SEQ ID NO: 23. In some aspects, the variant complementarity determining region heavy chain 3 (CDRH3) comprises one or two conservative amino acid substitutions at positions 97-119 of SEQ ID NO: 23.

In some aspects, the antibodies disclosed herein can be recombinantly engineered, chimerized, or humanized. In some aspects, the antibodies disclosed herein can be affinity matured or human antibodies. In some aspects, the antibodies disclosed herein can be a Fab, an Fab′, an F(ab′)2, a Fv, a scFv, a diabody or fragments thereof. In some aspects, the antibody can be a monoclonal antibody. In some aspects, the monoclonal antibodies can be humanized or chimeric forms thereof. In some aspects, the monoclonal antibody can be a humanized antibody. By known means and as described herein, polyclonal or monoclonal antibodies, antibody fragments, binding domains and CDRs (including engineered forms of any of the foregoing) may be created that are specific for SLO antigen, one or more of its respective epitopes, or conjugates of any of the foregoing, whether such antigens or epitopes are isolated from natural sources or are synthetic derivatives or variants of the natural protein.

A monoclonal antibody is a single, clonal species of antibody wherein every antibody molecule recognizes the same epitope because all antibody producing cells are derived from a single, antibody-producing B-lymphocyte (or other clonal cell, such as a cell that recombinantly expresses the antibody molecule). The methods for generating monoclonal antibodies (MAbs) generally begin along the same lines as those for preparing polyclonal antibodies. In some aspects, rodents such as mice and rats are used in generating monoclonal antibodies. In some aspects, rabbit, sheep, or frog cells are used in generating monoclonal antibodies. The use of rats is well known and may provide certain advantages. Mice (e.g., BALB/c mice) are routinely used and generally give a high percentage of stable fusions. Hybridoma technology as used in monoclonal antibody production can involve, for example, the fusion of a single, antibody-producing B lymphocyte isolated from a mouse previously immunized with a SLO protein or peptide with an immortalized cell, e.g., a mouse cell line. This technology provides a method to propagate a single antibody-producing cell for an indefinite number of generations, such that unlimited quantities of structurally identical antibodies having the same antigen or epitope specificity, i.e., monoclonal antibodies, may be produced.

Methods have been developed to replace light and heavy chain constant domains of the monoclonal antibody with analogous domains of human origin, leaving the variable regions of the foreign antibody intact. Alternatively, “fully human” monoclonal antibodies are produced in mice or rats that are transgenic for human immunoglobulin genes. Methods have also been developed to convert variable domains of monoclonal antibodies to more human form by recombinantly constructing antibody variable domains having both rodent and human amino acid sequences. In “humanized” monoclonal antibodies, only the hypervariable CDRs are derived from non-human (e.g., mouse, rat, chicken, llama, etc.) monoclonal antibodies, and the framework regions are derived from human antibody amino acid sequences. The replacement of amino acid sequences in the antibody that are characteristic of rodents with amino acid sequences found in the corresponding positions of human antibodies reduces the likelihood of adverse immune reaction to foreign protein during therapeutic use in humans. A hybridoma or other cell producing an antibody may also be subject to genetic mutation or other changes, which may or may not alter the binding specificity of antibodies produced by the hybridoma.

Engineered antibodies may be created using monoclonal and other antibodies and recombinant DNA technology to produce other antibodies or chimeric molecules that retain the antigen or epitope binding specificity of the original antibody, i.e., the molecule has a specific binding domain. Such techniques may involve introducing DNA encoding the immunoglobulin variable region or the CDRs of an antibody into the genetic material for the framework regions, constant regions, or constant regions plus framework regions, of a different antibody. See, for instance, U.S. Pat. Nos. 5,091,513 and 6,881,557, which are incorporated herein by reference.

By known means as described herein, polyclonal or monoclonal antibodies, antibody fragments having binding activity, binding domains and CDRs (including engineered forms of any of the foregoing), may be created that specifically bind to SLO protein, one or more of its respective epitopes, or conjugates of any of the foregoing, whether such antigens or epitopes are isolated from natural sources or are synthetic derivatives or variants of the natural compounds.

Antibodies may be produced from any animal source, including birds and mammals. In some aspects, the antibodies can be ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference. These techniques are further described in Marks et al., 1992, Bio/Technol., 10:779-783; Stemmer, 1994, Nature, 370:389-391; Gram et al., 1992, Proc. Natl. Acad. Sci. USA, 89:3576-3580; Barbas et al., 1994, Proc. Natl. Acad. Sci. USA, 91:3809-3813; and Schier et al., 1996, Gene, 169(2):147-155.

Methods for producing polyclonal antibodies in various animal species, as well as for producing monoclonal antibodies of various types, including humanized, chimeric, and fully human, are well known in the art and are highly reproducible. For example, the following U.S. patents provide descriptions of such methods and are herein incorporated by reference: U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; 6,891,024; 7,407,659; and 8,178,098.

In some aspects, the antibody can be a single chain antibody. In some aspects, the antibody can be linked to a detectable label. In some aspects, antibody can be a monovalent or a bivalent antibody.

In some aspects, the antibodies disclosed herein can be an IgG, an IgM, an IgA, an IgD, or an IgE antibody or antigen binding fragment thereof. In some aspects, the antibodies can be of the IgG, IgM, IgA, IgD, and IgE Ig classes or a genetically modified IgG class antibody, as well as polypeptides comprising one or more antibody CDR regions that retain antigen binding activity. In some aspects, the antibody can be an IgG class of antibody. In some aspects, the IgG class antibody can be an IgG1, IgG2, IgG3, or IgG4 class antibody.

In some aspects, the antibody can be a bispecific antibody. Unifying two antigen binding sites of different specificity into a single construct, bispecific antibodies have the ability to bring together two discreet antigens with exquisite specificity and therefore have great potential as therapeutic agents. Bispecific antibodies were originally made by fusing two hybridomas, each capable of producing a different immunoglobulin. Bispecific antibodies can also be produced by joining two scFv antibody fragments while omitting the Fc portion present in full immunoglobulins. Each scFv unit in such constructs can contain one variable domain from each of the heavy (V_{H}) and light (V_{L}) antibody chains, joined with one another via a synthetic polypeptide linker, the latter often being genetically engineered so as to be minimally immunogenic while remaining maximally resistant to proteolysis. Respective scFv units may be joined by a number of known techniques, including incorporation of a short (usually less than 10 amino acids) polypeptide spacer bridging the two scFv units, thereby creating a bispecific single chain antibody. The resulting bispecific single chain antibody is therefore a species containing two V_{H}/V_{L} pairs of different specificity on a single polypeptide chain, in which the V_{H} and V_{L} domains in a respective scFv unit are separated by a polypeptide linker long enough to allow intramolecular association between these two domains, such that the so-formed scFv units are contiguously tethered to one another through a polypeptide spacer kept short enough to prevent unwanted association between, for example, the V_{H} domain of one scFv unit and the V_{L} of the other scFv unit.

Examples of antibody fragments suitable for use include, without limitation: (i) the Fab fragment, consisting of V_{L}, V_{H}, C_{L}, and C_{H1} domains; (ii) the “Fd” fragment consisting of the V_{H} and C_{H1} domains; (iii) the “Fv” fragment consisting of the V_{L} and V_{H} domains of a single antibody; (iv) the “dAb” fragment, which consists of a V_{H} domain; (v) isolated CDR regions; (vi) F(ab′)2 fragments, a bivalent fragment comprising two linked Fab fragments; (vii) single chain Fv molecules (“scFv”), in which a V_{H} domain and a V_{L} domain are linked by a peptide linker that allows the two domains to associate to form a binding domain; (viii) bi-specific single chain Fv dimers (see U.S. Pat. No. 5,091,513); and (ix) diabodies, multivalent, or multispecific fragments constructed by gene fusion (U.S. Patent Appln. Pub. No. 20050214860). Fv, scFv, or diabody molecules may be stabilized by the incorporation of disulfide bridges linking the V_{H} and V_{L} domains. Minibodies comprising a scFv joined to a C_{H3} domain (Hu et al., 1996, Cancer Res., 56:3055-3061) may also be useful. In addition, antibody-like binding peptidomimetics are also contemplated. “Antibody like binding peptidomimetics” (ABiPs), which are peptides that act as pared-down antibodies and have certain advantages of longer serum half-life as well as less cumbersome synthesis methods, have been reported by Liu et al., 2003, Cell Mol. Biol., 49:209-216.

In some aspects, animals may be inoculated with an antigen, such as a SLO polypeptide or peptide to generate an immune response and produce antibodies specific for the SLO polypeptide. Frequently, an antigen is bound or conjugated to another molecule to enhance the immune response. As used herein, a conjugate can be any peptide, polypeptide, protein, or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

The antibodies described herein directed to SLO will have the ability to neutralize, block, inhibit, or counteract the effects of SLO (e.g., inhibit hemolysis) regardless of the animal species, monoclonal cell line or other source of the antibody. Certain animal species may be less preferable for generating therapeutic antibodies because they may be more likely to cause an immune or allergic response due to activation of the complement system through the “Fc” portion of the antibody. However, whole antibodies may be enzymatically digested into the “Fc” (complement binding) fragment, and into peptide fragments having the binding domains or CDRs. Removal of the Fc portion reduces the likelihood that this antibody fragment will elicit an undesirable immunological response and, thus, antibodies without an Fc portion may be preferential for prophylactic or therapeutic treatments. As described above, antibodies may also be constructed so as to be chimeric, humanized, or partially or fully human, so as to reduce or eliminate potential adverse immunological effects resulting from administering to an animal an antibody that has been produced in, or has amino acid sequences from, another species.

In some aspects, the antibodies disclosed herein bind to human SLO and do not cross-react with mouse SLO. In some aspects, the antibody binds to SLO with an affinity of greater than or equal to 40 pM. In some aspects, the antibody binds to SLO with affinity of about 40 pM to 80 pM. In some aspects, the antibody selectively binds to SLO and inhibits binding of SLO to host cell membranes via cholesterol. In some aspects, the antibody selectively binds to SLO and inhibits binding of SLO to a cholesterol-containing host cell membrane. In some aspects, the antibody selectively binds to SLO and inhibits binding of SLO to host cell membranes via carbohydrates. In some aspects, the antibody selectively binds to SLO and inhibits binding of SLO to a carbohydrate-containing host cell membrane. In some aspects, the antibody selectively binds to SLO and inhibits binding of SLO to eukaryotic cells that promote cytolysis. In some aspects, the antibody selectively binds to human SLO and inhibits binding of human SLO to human eukaryotic cells that promote cytolysis.

The term “specifically binds” (or “immunospecifically binds”) is not intended to indicate that an antibody binds exclusively to its intended target. Rather, an antibody “specifically binds” if its affinity for its intended target is about, for example, 5-fold greater when compared to its affinity for a non-target molecule. Suitably there is no significant cross-reaction or cross-binding with undesired substances. The affinity of the antibody will, for example, be at least about 5-fold, such as 10-fold, such as 25-fold, especially 50-fold, and particularly 100-fold or more, greater for a target molecule than its affinity for a non-target molecule. In some aspects, specific binding between an antibody or other binding agent and an antigen means a binding affinity of at least 10⁶ M-¹. Antibodies may, for example, bind with affinities of at least about 10⁷ M-¹, such as between about 10⁸ M-¹ to about 10⁹ M-¹, about 10⁹ M-¹ to about 10¹⁰ M-¹, or about 10-¹⁰M-¹ to about 10¹¹ M-¹. Antibodies may, for example, bind with an EC50 of 80 pM or less, 70 pM or less, 60 pM or less, 50 pM or less, or more preferably 40 pM or less. In some aspects, the antibodies can bind with an EC50 of about 30 pM, 25 pM, 20 pM, 15 pM, 10 pM, or 5 pM or less.

In some aspects, the antibodies described herein comprise a heavy chain variable region, wherein the heavy chain variable region comprises one or more complementarity determining region (CDRHs) CDRH1, CDRH2 and CDRH3 with amino acid sequences that have 0, 1, 2, 3, 4, or 5 conservative amino acid substitutions in 1, 2 or 3 CDRHs having the amino acid sequences of SEQ ID NO: 4, SEQ ID NO: 5, and SEQ ID NO: 6, respectively; and/or a light chain variable region comprising one or more complementarity determining region (CDRLs) CDRL1, CDRL2 and CDRL3 with the amino acid sequences that have 0, 1, 2, 3, 4, or 5 conservative amino acid substitutions in 1, 2 or 3 CDRLs having the amino acid sequences of SEQ ID NO: 1, SEQ ID NO: 2, and SEQ ID NO: 3, respectively.

In some aspects, the antibodies described herein comprise a heavy chain variable region, wherein the heavy chain variable region comprises one or more complementarity determining region (CDRHs) CDRH1, CDRH2 and CDRH3 with amino acid sequences that have 0, 1, 2, 3, 4, or 5 conservative amino acid substitutions in 1, 2 or 3 CDRHs having the amino acid sequences of SEQ ID NO: 12, SEQ ID NO: 13, and SEQ ID NO: 14, respectively; and/or a light chain variable region comprising one or more complementarity determining region (CDRLs) CDRL1, CDRL2 and CDRL3 with the amino acid sequences that have 0, 1, 2, 3, 4, or 5 conservative amino acid substitutions in 1, 2 or 3 CDRLs having the amino acid sequences of SEQ ID NO: 9, SEQ ID NO: 10, and SEQ ID NO: 11, respectively.

In some aspects, the antibodies described herein comprise a heavy chain variable region, wherein the heavy chain variable region comprises one or more complementarity determining region (CDRHs) CDRH1, CDRH2 and CDRH3 with amino acid sequences that have 0, 1, 2, 3, 4, or 5 conservative amino acid substitutions in 1, 2 or 3 CDRHs having the amino acid sequences of SEQ ID NO: 19, SEQ ID NO: 20, and SEQ ID NO: 21, respectively; and/or a light chain variable region comprising one or more complementarity determining region (CDRLs) CDRL1, CDRL2 and CDRL3 with the amino acid sequences that have 0, 1, 2, 3, 4, or 5 conservative amino acid substitutions in 1, 2 or 3 CDRLs having the amino acid sequences of SEQ ID NO: 17, SEQ ID NO: 2, and SEQ ID NO: 18, respectively.

Disclosed herein are isolated antibodies or antibody fragments thereof comprising a light chain variable region amino acid sequence of SEQ ID NO: 7 and a heavy chain variable region amino acid sequence of SEQ ID NO: 8, wherein the isolated antibody comprises 1, 2, 3, 4, or 5 conservative amino acid substitutions in the light or heavy chain variable region amino acid sequences.

Disclosed herein are isolated antibodies or antibody fragments thereof comprising a light chain variable region amino acid sequence of SEQ ID NO: 14 and a heavy chain variable region amino acid sequence of SEQ ID NO: 15, wherein the isolated antibody comprises 1, 2, 3, 4, or 5 conservative amino acid substitutions in the light or heavy chain variable region amino acid sequences.

Disclosed herein are isolated antibodies or antibody fragments thereof comprising a light chain variable region amino acid sequence of SEQ ID NO: 22 and a heavy chain variable region amino acid sequence of SEQ ID NO: 23, wherein the isolated antibody comprises 1, 2, 3, 4, or 5 conservative amino acid substitutions in the light or heavy chain variable region amino acid sequences.

In some aspects, the antibodies disclosed herein can specifically bind to an epitope of SLO. In some aspects, the epitope of SLO can be ENFVPKEGVKKADKFI (SEQ ID NO: 24).

In some aspects, the antibodies disclosed herein can prevent, inhibit or block SLO binding to a target host cell membrane. In some aspects, the target host cell membrane can be a cholesterol-containing host cell membrane, a carbohydrate-containing host cell membrane, or an eukaryotic cells that promote cytolysis. In some aspects, the antibodies disclosed herein can inhibit the binding of human SLO to human target host cell membrane. In some aspects, the antibodies disclosed herein can prevent or inhibit red blood cell hemolysis, and/or prevent or inhibit epithelial cell cytotoxicity. In some aspects, the antibodies disclosed herein can inhibit or prevent streptolysin O-induced RBC-hemolysis.

Antibody proteins may be recombinant, or synthesized in vitro. It is contemplated that in anti-SLO antibody-containing compositions as described herein can comprise between about 0.001 mg and about 10 mg of total antibody polypeptide per ml. Thus, the concentration of antibody protein in a composition can be about, at least about or at most about or equal to 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, at most about, or equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds SLO.

Disclosed herein are compositions comprising any of the antibodies or isolated antibodies described herein. In some aspects, the compositions can further comprise at least one pharmaceutically acceptable carrier or diluent.

In some aspects, the compositions described herein can comprise a detectable label or reporter. An antibody or an immunological portion of an antibody that retains binding activity, can be chemically conjugated to, or recombinantly expressed as, a fusion protein with other proteins. For the purposes as described herein, all such fused proteins are included in the definition of antibodies or an immunological portion of an antibody. In some aspects, antibodies and antibody-like molecules generated against SLO or polypeptides that are linked to at least one agent to form an antibody conjugate or payload are encompassed. In order to increase the efficacy of antibody molecules as diagnostic or therapeutic agents, it is conventional to link or covalently bind or complex at least one desired molecule or moiety to the antibody. Such a linked molecule or moiety may be, but is not limited to, at least one effector, detectable label or reporter molecule. Effector molecules comprise molecules having a desired activity, e.g., cytotoxic activity. Non-limiting examples of effector molecules that may be attached to antibodies include toxins, therapeutic enzymes, antibiotics, radio-labeled nucleotides and the like. By contrast, a reporter molecule or detectable label is defined as any moiety that may be detected using an assay. Non-limiting examples of reporter molecules and detectable labels that can be conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin, and the like. Several methods are known in the art for attaching or conjugating an antibody to a conjugate molecule or moiety. Some attachment methods involve the use of a metal chelate complex, employing by way of nonlimiting example, an organic chelating agent such a diethylenetriaminepentaacetic acid anhydride (DTPA); ethylenetriaminetetraacetic acid; N-chloro-p-toluenesulfonamide; and/or tetrachloro-3-6α-diphenylglycouril-3 attached to the antibody. Antibodies, particularly the monoclonal antibodies as described herein, may also be reacted with an enzyme in the presence of a coupling agent such as glutaraldehyde or periodate. Conjugates with fluorescein markers are conventionally prepared in the presence of these coupling agents or by reaction with an isothiocyanate. In some aspects, an anti-SLO antibody as described herein, particularly a binding fragment thereof, may be coupled or linked to a compound or substance, such as polyethylene glycol (PEG), to increase its in vivo half-life in plasma, serum, or blood following administration.

In some aspects, the antibodies described herein can be specifically bind to their intended target. In some aspects, the antibodies described herein have no off site binding.

Also disclosed herein are vectors comprising a sequence of encoding any of the isolated antibodies or any of the antibody fragments described herein.

### Methods

Disclosed herein are methods for treating a necrotizing soft tissue infection in a subject. The methods can comprise administering to the subject a therapeutically effective amount of any of the isolated antibodies described herein or any of the compositions described herein. In some aspects, the necrotizing soft tissue infection can be fasciitis, myositis or cellulitis. In some aspects, the necrotizing soft tissue infection can be a skin/dermal infection or strep throat.

Disclosed herein are methods of inhibiting or preventing streptolysin O-induced RBC-hemolysis in a subject. In some aspects, the methods can comprise administering to the subject (e.g., a subject with a necrotizing soft tissue infection) a therapeutically effective amount of any of the isolated antibodies described herein or any of the composition disclosed herein. In some aspects, the subject has a necrotizing soft tissue infection.

Disclosed herein are methods of preventing or inhibiting cytotoxicity in epithelial cells in a subject. In some aspects, the methods can comprise administering to the subject (e.g., a subject with a necrotizing soft tissue infection) a therapeutically effective amount of any of the isolated antibodies described herein or any of the composition disclosed herein. In some aspects, the subject has a necrotizing soft tissue infection.

Disclosed herein are methods of reducing SLO levels associated with a necrotizing soft tissue infection in a subject. In some aspects, the SLO levels can be increased compared to a reference sample or control subject. In some aspects, the method comprises administering therapeutically effective amount of any of the isolated antibodies disclosed herein or any of the compositions disclosed herein. In some aspects, the method comprises administering therapeutically effective amount of any of the anti-SLO antibodies disclosed herein. In some aspects, the therapeutically effective amount of the anti-SLO antibodies can be an amount to form a complex between the anti-SLO antibody and SLO in the subject, thereby inducing reducing SLO levels associated with a necrotizing soft tissue infection in the subject.

In some aspects, the subject can be identified in need of treatment before the administering step. In some aspects, the antibody can be administered in a pharmaceutically acceptable composition. In some aspects, the antibody can be administered systemically, intravenously, intradermally, intramuscularly, intraperitoneally, subcutaneously or locally into inflamed tissues or organs.

In some aspects, the methods can further comprising administering one or more drugs or therapeutic agents to the subject. Examples of drugs or therapeutic agents that can be administered in combination with any of the antibodies described herein include but are not limited to imipenem, meropenem, ertapenem, piperacillin (tazobactam), tigecycline, penicillin, clindamycin, a fluoroquinolone or an aminoglycoside, vancomycin, daptomycin, linezolid, azithromycin, or erythromycin. In some aspects, one or more macrolides (e.g., azithromycin and erythromycin), glycopeptides (e.g., vancomycin), lipopeptides (e.g., daptomycin), carbapenems (e.g., imipenem) or tetracyclines (e.g., tigecycline) can be administered in combination with any of the antibodies described herein

Treatment of diseases. Disclosed herein are antibodies or antigen binding fragments thereof, as described herein (e.g., an antibody that specifically and preferentially binds to SLO and blocks or inhibits binding of SLO to target host cell membrane) that can be used in treatment methods and administered to treat or prevent an necrotizing soft tissue infection, streptolysin O-induced RBC-hemolysis, or cytotoxicity in epithelial cells. Accordingly, provided herein are methods of treating a necrotizing soft tissue infection, in a subject having necrotizing soft tissue infection. In some aspects, the methods can comprise administering to a subject a therapeutically effective amount of any of the antibodies described herein or any of the compositions comprising at least one of antibodies as described herein. In some aspects, the drug or therapeutic agent can be an anti-SLO antibody or a composition comprising at least one anti-SLO antibody.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with or diagnosed with a necrotizing soft tissue infection, in an amount sufficient to at least partially improve a sign or symptom or to inhibit the progression of (and preferably arrest) the symptoms of the disease or condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the necrotizing soft tissue infection is delayed, hindered, or prevented, or the necrotizing soft tissue infection or a symptom of the necrotizing soft tissue infection is ameliorated or its frequency can be reduced. One or more of the symptoms can be less severe. Recovery can be accelerated in an individual who has been treated. For example, treatment of necrotizing soft tissue infection may involve, for example, preventing or reducing RBC-hemolysis; preventing or inhibiting cytotoxicity in epithelial cells; or preventing, inhibiting or reducing skin, muscle or other soft tissue death or destruction, pain, wound with fever, skin warmth or skin redness, skin blisters, pus-filled bumps, or numbness.

Treatment of these subjects with an effective amount of at least one of the anti-SLO antibodies as described herein can result in binding of one or more of the disclosed antibodies to SLO, thereby preventing, blocking or inhibiting SLO from binding to its target host cell membrane, and thereby preventing or inhibiting streptolysin O-induced RBC-hemolysis or epithelial cell cytoxicity. Accordingly, the methods as provided are advantageous for a subject who is in need of, capable of benefiting from, or who is desirous of receiving the benefit of, the anti-infection results, or the amelioration of one or more necrotizing soft tissue infection symptoms, achieved by the practice of the present methods. A subject's seeking the therapeutic benefits of the methods involving administration of at least one anti-SLO antibody in a therapeutically effective amount, or receiving such therapeutic benefits offer advantages to the art. In addition, the present methods offer the further advantages of eliminating or avoiding side effects, adverse outcomes, contraindications, and the like, or reducing the risk or potential for such issues to occur compared with other treatments and treatment modalities.

Necrotizing soft tissue infections for which the present methods are useful include but are not limited to fasciitis, myositis or cellulitis.

Therapeutic compositions comprising antibodies are conventionally administered intravenously, such as by injection of a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required diluent, i.e., carrier, or vehicle. The compositions comprising any of the anti-SLO antibodies disclosed herein can be administered in a manner compatible with the dosage formulation, and in a therapeutically effective amount. The quantity to be administered depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimens for initial and booster administration are also contemplated and may typically involve an initial administration followed by repeated doses at one or more intervals (hours) by a subsequent injection or other administration. In some aspects, multiple administrations can be suitable for maintaining continuously high serum and tissue levels of antibody. Alternatively, continuous intravenous infusion sufficient to maintain concentrations in the blood in the ranges specified for in vivo therapies are contemplated.

It is contemplated that an anti-SLO antibody as described herein can be administered systemically or locally to treat infection and/or disease, such as to inhibit or prevent streptolysin O-induced RBC-hemolysis or to prevent or inhibit cytotoxicity in epithelial cells in infected patients with locally or advanced necrotizing soft tissue infections or at risk for necrotizing soft tissue infections. The antibodies can be administered alone or in combination with antibiotic drugs or antifungal drugs. In some aspects, the anti-SLO antibodies can be administered to reduce the infection spread in the patient prior to surgery or other procedures. Alternatively, they can be administered at periodic intervals after surgery. As noted herein, a therapeutically effective amount of an antibody can be a predetermined amount calculated to achieve the desired effect. Thus, the dosage ranges for the administration of an anti-SLO antibody are those large enough to produce the desired effect in which the symptoms of necrotizing soft tissue infection are reduced. Optimally, the dosage should not be so large as to cause adverse side effects. Generally, the dosage will vary with age of, condition of, size and gender of, and extent of the disease in the subject or patient and can be determined by one of skill in the art such as a medical practitioner or clinician. Of course, the dosage may be adjusted by the individual physician in the event of any complication.

Treatment methods. In some aspects, the compositions and methods as described herein comprise the administration of an anti-SLO antibody as described herein, alone, or in combination with a second or additional drug or therapy. Such drug or therapy may be applied in the treatment of any disease that is associated with SLO, and in some aspects, the interaction of human SLO with a target host cell membrane. For example, the disease can be a necrotizing soft tissue infection. The compositions and methods described herein can comprise at least one anti-SLO antibody that preferentially binds to SLO protein and has a therapeutic or protective effect in the treatment of a necrotizing soft tissue infection, particularly by preventing, reducing, blocking, or inhibiting the SLO/target host cell membrane interaction, thereby providing a therapeutic effect and treatment.

The compositions and methods, including combination therapies, have a therapeutic or protective effect and may enhance the therapeutic or protective effect, and/or increase the therapeutic effect of another drug, therapy or therapeutic agent (e.g., antibiotic).

Therapeutic and prophylactic methods and compositions can be provided in a combined amount effective to achieve the desired effect, such as inhibiting or preventing streptolysin O-induced RBC-hemolysis and/or preventing or inhibiting cytotoxicity in epithelial cells; reducing one or more symptoms of a necrotizing soft tissue infection, reducing SLO levels. This process may involve administering an anti-SLO antibody or a binding fragment thereof and a second therapy. The second therapy may or may not have a direct cytotoxic effect. A tissue, tumor, and/or cell can be exposed to one or more compositions or pharmacological formulation(s) comprising one or more of the agents (e.g., an antibody or antibiotic), or by exposing the tissue, and/or cell with two or more distinct compositions or formulations, wherein one composition provides, for example, 1) an antibody, 2) an antibiotic, 3) both an antibody and an antibiotic, 4) two or more antibodies, or 5) two or more antibodies and one or more antibiotics. In some aspects, the second therapy can be also an anti-SLO antibody. Also, it is contemplated that such a combination therapy can be used in conjunction with surgical therapy (e.g., surgical debridement) or antifungal treatments, hyperbaric oxygen therapy or tetanus vaccine.

By way of example, the terms “contacted” and “exposed,” when applied to a cell, are used herein to describe a process by which a therapeutic polypeptide, for example, an anti-SLO antibody as described herein, is delivered to a target cell or is placed in direct juxtaposition with the target cell, particularly to bind specifically to the target antigen, e.g., SLO. Such binding by a therapeutic anti-SLO antibody or binding fragment thereof prevents, blocks, inhibits, or reduces the interaction of SLO with a target host cell membrane (e.g., a cholesterol-containing host cell membrane or a carbohydrate-containing host cell membrane). In some aspects, an antibiotic agent or an antifungal agent can also be administered or delivered to the subject in conjunction with the anti-SLO antibody or binding fragment thereof. To achieve SLO neutralization, for example, one or more agents can be delivered to a cell in a combined amount effective to kill the bacteria or prevent it from producing SLO.

Any of the anti-SLO antibodies disclosed herein may be administered before, during, after, or in various combinations relative to another treatment (e.g., antibiotic, or antifungal agent). The administrations may be in intervals ranging from concurrently to minutes to days to weeks before or after one another. In some aspects, in which the antibody is provided to a patient separately from an antibiotic agent or antifungal agent, it would be generally ensured that a significant period of time did not expire between the time of each delivery, such that the administered compounds would still be able to exert an advantageously combined effect for the patient. Illustratively, in such instances, it is contemplated that one may provide a patient with the antibody and the antibiotic therapy or antifungal agent within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In some aspects, a course of treatment or treatment cycle will last 1-90 days or more (this range includes intervening days and the last day). It is contemplated that one agent may be given on any day of day 1 to day 90 (this such range includes intervening days and the last day) or any combination thereof, and another agent is given on any day of day 1 to day 90 (this such range includes intervening days and the last day) or any combination thereof. Within a single day (24-hour period), the patient may be given one or multiple administrations of the agent(s). Moreover, after a course of treatment, it is contemplated that there may be a period of time at which no second agent (e.g., antibiotic treatment or antifungal agent) is administered. This time period may last, for example, for 1-7 days, and/or 1-5 weeks, and/or 1-12 months or more (this such range includes intervening days and the upper time point), depending on the condition of the patient, such as prognosis, strength, health, etc. Treatment cycles would be repeated as necessary. Various combinations of treatments may be employed. In the representative examples of combination treatment regimens shown below, an antibody, such as an anti-SLO antibody or binding fragment thereof is represented by “A” and an antibiotic therapy is represented by “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A.

Administration of any antibody or therapy as described herein to a patient will follow general protocols for the administration of such compounds, taking into account the toxicity, if any, of the agents. Therefore, in some aspects there is a step of monitoring adverse events and toxicity, particularly those that may be attributable to combination therapy.

In some aspects, methods are disclosed comprising administering an anti-SLO antibody alone or in combination with another agent (e.g., antibiotic agent or antifungal agent) to a subject in need thereof, i.e., a subject with a necrotizing soft tissue infection). Prior to administration of the anti-SLO antibody, a sample of the subject's blood or one or more symptoms associated with the necrotizing soft tissue infection may be evaluated for the presence or level of SLO. If the results of such an evaluation reveals that the subject's symptoms associated with the necrotizing soft tissue infection is positive for SLO (or SLO antibodies) or the level of SLO (or SLO antibodies) is increased compared to a reference sample or prior sample from the same subject, the subject would be selected for treatment based on the likelihood that subject's SLO+disease state or condition would be more amenable to treatment with the anti-SLO antibody and treatment may proceed with a more likely beneficial outcome. A medical professional or physician may advise the subject to proceed with the anti-SLO antibody treatment method, and the subject may decide to proceed with treatment based on the advice of the medical professional or physician. In addition, during the course of treatment, the subject's blood may be assayed for the presence of SLO (or SLO antibodies) as a way to monitor the progress or effectiveness of treatment. If the assay shows a change, loss, or decrease, for example, in SLO in the subject's blood, a decision may be taken by the medical professional in conjunction with the subject as to whether the treatment should continue or be altered in some fashion, e.g., a higher dosage, the addition of another anti-SLO agent or therapy or antibiotic or antifungal agent, and the like.

Antibiotic therapy. A wide variety of antibiotic agents may be used in accordance with the treatment or therapeutic methods as described herein. The term “antibiotic” refers to the use of drugs to treat bacterial infections or inhibit the growth of or destroy microorganisms. An “antibiotic agent” connotes a compound or composition that is administered in the treatment (and prevention) of bacterial infections. Such agents or drugs are categorized by their mode of activity within a cell, for example, whether and at what stage they kill or inhibit the growth of bacteria. Alternatively, an antibiotic agent may be characterized based on its ability to be selective against a particular type of pathogen or a broad class of pathogens.

Nonlimiting examples of antibiotic agents include imipenem, meropenem, ertapenem, piperacillin (tazobactam), tigecycline, penicillin, clindamycin, a fluoroquinolone or an aminoglycoside, vancomycin, daptomycin, linezolid, azithromycin, or erythromycin, and pharmaceutically acceptable salts, acids, or derivatives of any of the above.

Protein Purification. Protein, including antibody and, particularly, anti-SLO antibody, purification techniques are well known to those of skill in the art. These techniques involve, at one level, the homogenization and crude fractionation of the cells, tissue, or organ into polypeptide and non-polypeptide fractions. The protein or polypeptide of interest may be further purified using chromatographic and electrophoretic techniques to achieve partial or complete purification (or purification to homogeneity) unless otherwise specified. Analytical methods particularly suited to the preparation of a pure protein or peptide are ion-exchange chromatography, size-exclusion chromatography, reverse phase chromatography, hydroxyapatite chromatography, polyacrylamide gel electrophoresis, affinity chromatography, immunoaffinity chromatography, and isoelectric focusing. A particularly efficient method of purifying peptides is fast-performance liquid chromatography (FPLC) or even high-performance liquid chromatography (HPLC). As is generally known in the art, the order of conducting the various purification steps may be changed, and/or certain steps may be omitted, and still result in a suitable method for the preparation of a substantially purified polypeptide.

A purified polypeptide refers to a polypeptide which is isolatable or isolated from other components and purified to any degree relative to its naturally-obtainable state. An isolated or purified polypeptide, therefore, also refers to a polypeptide free from the environment in which it may naturally occur, e.g., cells, tissues, organs, biological samples, and the like. Generally, “purified” will refer to a polypeptide composition that has been subjected to fractionation to remove various other components, and which composition substantially retains its expressed biological activity. A “substantially purified” composition refers to one in which the polypeptide forms the major component of the composition, and as such, constitutes about 50%, about 60%, about 70%, about 80%, about 90%, about 95%, or more of the protein component of the composition.

Various methods for quantifying the degree of purification of polypeptides, such as antibody proteins, are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “fold purification number.” The actual units used to represent the amount of activity will, of course, be dependent upon the particular assay technique chosen to follow the purification, and whether or not the expressed polypeptide exhibits a detectable activity.

There is no general requirement that the polypeptide will always be provided in its most purified state. Indeed, it is contemplated that less substantially purified products may have utility in some aspects. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “fold” purification than the same technique utilizing a low pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance (protein) to be isolated and a molecule to which it can specifically bind, e.g., a receptor-ligand type of interaction. The column material (resin) is synthesized by covalently coupling one of the binding partners to an insoluble matrix. The column material is then able to specifically adsorb the substance from the solution that is passed over the column resin. Elution occurs by changing the conditions to those in which binding will be disrupted/will not occur (e.g., altered pH, ionic strength, temperature, etc.). The matrix should be a substance that does not adsorb molecules to any significant extent and that has a broad range of chemical, physical, and thermal stability. The ligand should be coupled in such a way as to not affect its binding properties. The ligand should also provide relatively tight binding; however, elution of the bound substance should occur without destroying the sample protein desired or the ligand.

Size-exclusion chromatography (SEC) is a chromatographic method in which molecules in solution are separated based on their size, or in more technical terms, their hydrodynamic volume. It is usually applied to large molecules or macromolecular complexes, such as proteins and industrial polymers. Typically, when an aqueous solution is used to transport the sample through the column, the technique is known as gel filtration chromatography, versus the name gel permeation chromatography, which is used when an organic solvent is used as a mobile phase. The underlying principle of SEC is that particles of different sizes will elute (filter) through a stationary phase at different rates, resulting in the separation of a solution of particles based on size. Provided that all of the particles are loaded simultaneously or near simultaneously, particles of the same size should elute together.

High-performance (aka high-pressure) liquid chromatography (HPLC) is a form of column chromatography used frequently in biochemistry and analytical chemistry to separate, identify, and quantify compounds. HPLC utilizes a column that holds chromatographic packing material (stationary phase), a pump that moves the mobile phase(s) through the column, and a detector that shows the retention times of the molecules. Retention time varies depending on the interactions between the stationary phase, the molecules being analyzed, and the solvent(s) used

Pharmaceutical Preparations. Where clinical application of a pharmaceutical composition comprising an anti-SLO antibody is undertaken, it is generally beneficial to prepare a pharmaceutical or therapeutic composition appropriate for the intended application. In general, pharmaceutical compositions can comprise an effective amount of one or more polypeptides or additional agents dissolved or dispersed in a pharmaceutically acceptable carrier. In some aspects, pharmaceutical compositions may comprise, for example, at least about 0.1% of a polypeptide or antibody. In some aspects, a polypeptide or antibody may comprise between about 2% to about 75% of the weight of the unit, or between about 25% to about 60%, for example, and any range derivable there between, including the upper and lower values. The amount of active compound(s) in each therapeutically useful composition may be prepared in such a way that a suitable dosage will be obtained in any given unit dose. Factors, such as solubility, bioavailability, biological half-life, route of administration, product shelf life, as well as other pharmacological considerations, are contemplated by one skilled in the art of preparing such pharmaceutical formulations, and as such, a variety of dosages and treatment regimens may be desirable.

Further in some aspects, the composition suitable for administration can be provided in a pharmaceutically acceptable carrier with or without an inert diluent. The carrier should be assimilable and include liquid, semi-solid, e.g., gels or pastes, or solid carriers. Examples of carriers or diluents include but are not limited to fats, oils, water, saline solutions, lipids, liposomes, resins, binders, fillers, and the like, or combinations thereof. As used herein, “pharmaceutically acceptable carrier” includes any and all aqueous solvents (e.g., water, alcoholic/aqueous solutions, ethanol, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings (e.g., lecithin), surfactants, antioxidants, preservatives (e.g., antibacterial or antifungal agents, anti-oxidants, chelating agents, inert gases, parabens (e.g., methylparabens, propylparabens), chlorobutanol, phenol, sorbic acid, thimerosal), isotonic agents (e.g., sugars, sodium chloride), absorption delaying agents (e.g., aluminum monostearate, gelatin), salts, drugs, drug stabilizers (e.g., buffers, amino acids, such as glycine and lysine, carbohydrates, such as dextrose, mannose, galactose, fructose, lactose, sucrose, maltose, sorbitol, mannitol, etc.), gels, binders, excipients, disintegration agents, lubricants, sweetening agents, flavoring agents, dyes, fluid and nutrient replenishers, such like materials and combinations thereof, as would be known to one of ordinary skill in the art. Except insofar as any conventional media, agent, diluent, or carrier is detrimental to the recipient or to the therapeutic effectiveness of the composition contained therein, its use in an administrable composition for the practice of the methods is appropriate. The pH and exact concentration of the various components in a pharmaceutical composition are adjusted according to well-known parameters. In some aspects, the composition can be combined with the carrier in any convenient and practical manner, i.e., by solution, suspension, emulsification, admixture, encapsulation, absorption, grinding, and the like. Such procedures are routine for those skilled in the art.

In some aspects, the compositions may comprise different types of carriers depending on whether they are to be administered in solid, liquid, or aerosol form, and whether it needs to be sterile for the route of administration, such as injection. The compositions can be formulated for administration intravenously, intradermally, transdermally, intrathecally, intra-arterially, intraperitoneally, intranasally, intravaginally, intrarectally, intramuscularly, subcutaneously, mucosally, orally, topically, locally, by inhalation (e.g., aerosol inhalation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art. See, for example, Remington's Pharmaceutical Sciences, 18th Ed., 1990. Typically, such compositions can be prepared as either liquid solutions or suspensions; solid or reconstitutable forms suitable for use to prepare solutions or suspensions upon the addition of a liquid prior to injection can also be prepared; and, the preparations can also be emulsified.

The antibodies may be formulated into a composition in a free base, neutral, or salt form. Pharmaceutically acceptable salts include the acid addition salts, e.g., those formed with the free amino groups of a proteinaceous composition, or which are formed with inorganic acids, such as, for example, hydrochloric or phosphoric acids, or such organic acids as acetic, oxalic, tartaric, or mandelic acid. Salts formed with the free carboxyl groups may also be derived from inorganic bases, such as, for example, sodium, potassium, ammonium, calcium, or ferric hydroxides; or such organic bases as isopropylamine, trimethylamine, histidine, or procaine.

In some aspects, a pharmaceutical lipid vehicle composition that includes polypeptides, one or more lipids, and an aqueous solvent may be used. As used herein, the term “lipid” refers to any of a broad range of substances that are characteristically insoluble in water and extractable with an organic solvent. This broad class of compounds is well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether- and ester-linked fatty acids, polymerizable lipids, and combinations thereof. Of course, compounds other than those specifically described herein that are understood by one of skill in the art as lipids are also encompassed by the compositions and methods. One of ordinary skill in the art would be familiar with the range of techniques that can be employed for dispersing a composition in a lipid vehicle. For example, the antibody may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The term “unit dose” or “dosage” refers to physically discrete units suitable for use in a subject, each unit containing a predetermined quantity of the therapeutic antibody or composition containing the therapeutic antibody calculated to produce the desired responses discussed above in association with its administration, i.e., the appropriate route and treatment regimen. The quantity to be administered, both according to number of treatments and unit dose, depends on the effect desired. The actual dosage amount of a composition as described herein that can be administered to a patient or subject can be determined by physical and physiological factors, such as body weight, the age, health, and sex of the subject, the type of disease being treated, the extent of disease penetration, previous or concurrent therapeutic interventions, idiopathy of the subject, the route of administration, and the potency, stability, and toxicity of the particular therapeutic substance. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 milligram/kg/body weight or more per administration, and any range derivable therein. In non-limiting examples of a derivable range from the numbers listed herein, a range of about 5 milligram/kg/body weight to about 100 milligram/kg/body weight, about 5 microgram/kg/body weight to about 500 milligram/kg/body weight, etc., can be administered, based on the numbers described above. The foregoing doses include amounts between those indicated and are intended to also include the lower and upper values of the ranges. The practitioner responsible for administration will, in any event, determine the concentration of active ingredient(s) in a composition and appropriate dose(s) for the individual subject.

The particular nature of the therapeutic composition or preparation is not intended to be limiting. For example, suitable compositions may be provided in formulations together with physiologically tolerable liquid, gel, or solid carriers, diluents, and excipients. In some aspects, the therapeutic preparations may be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual subjects, as described supra.

SLO as a biomarker. Disclosed herein are methods comprising the use of at least one anti-SLO antibody or anti-SLO antibody fragment thereof as described herein. In some aspects, the methods can comprise detecting the amount of level of SLO in a sample obtained from a subject who has a necrotizing soft tissue infection or is exhibiting one or more symptoms of necrotizing soft tissue infection or any infection with where Streptococcus is the likely species. Such methods may be useful in biomarker evaluations of the level of SLO in a sample obtained from a subject who has a necrotizing soft tissue infection or is exhibiting one or more symptoms of a necrotizing soft tissue infection. For example, if the subject's sample is tested and determined to comprise a higher level of SLO compared to a reference sample, then the subject is a candidate for treatment with an anti-SLO antibody as described herein, alone, or in combination with another agent, for example, would benefit from the treatment. Such methods comprise obtaining a sample from a subject having a necrotizing soft tissue infection (or exhibiting one or more symptoms of a necrotizing soft tissue infection), testing the sample for the presence of SLO derived from the subject's sample using binding methods known and used in the art and as described herein, for example, using an anti-SLO antibody as described herein, and administering to the subject an effective amount of an anti-SLO antibody alone, or in combination with another agent, if the subject's sample is found to have a higher level of SLO when compared to a reference sample. Diagnosing the subject as having a necrotizing soft tissue infection or a necrotizing soft tissue infection prior to treatment allows for more effective treatment and benefit to the subject, as the administered anti-SLO antibody is more likely to block or inhibit the interaction of the subject's SLO with a cholesterol-containing host cell membrane or a carbohydrate-containing host cell membrane, thereby inhibiting or preventing streptolysin O-induced RBC-hemolysis or preventing or inhibiting cytotoxicity in epithelial cells in a subject. In some aspects, the methods can involve first selecting a subject whose exposure to a pathogen that is associated with a necrotizing soft tissue infection or disease state or condition may be amenable to testing for the presence of SLO levels.

Similar methods can be used to monitor the presence of SLO levels during a course of treatment or therapy, including combination treatments with an anti-SLO antibody and another antibiotic drug or treatment, over time, as well as after treatment has ceased. Such methods may also be used in companion diagnostic methods in which a treatment regimen or combination treatment, involves testing or assaying a sample obtained from the subject for SLO levels, prior to treatment and during the course of treatment, e.g., monitoring, to determine a successful outcome or the likelihood thereof.

### FUSIONS and CONJUGATES

The anti-SLO antibodies or polypeptides disclosed herein can also be expressed as fusion proteins with other proteins or chemically conjugated to another moiety. In some aspects, the antibodies or polypeptides can have an Fc portion that can be varied by isotype or subclass, can be a chimeric or hybrid, and/or can be modified, for example to improve effector functions, control half-life or tissue accessibility, augment biophysical characteristics, such as stability, and improve efficiency of production, which can be associated with cost reductions. Many modifications useful in the construction of fusion proteins and methods for making them are known in the art, for example, as reported by Mueller, J. P. et al., 1997, Mol. Immun. 34(6):441-452; Swann, P. G., 2008, Curr. Opin. Immunol., 20:493-499; and Presta, L. G., 2008, Curr. Opin. Immunol., 20:460-470. In some aspects, the Fc region can be the native IgG1, IgG2, or IgG4 Fc region of the antibody. In some aspects, the Fc region can be a hybrid, for example, a chimera containing IgG2/IgG4 Fc constant regions. Modifications to the Fc region include, but are not limited to, IgG4 modified to prevent binding to Fc gamma receptors and complement; IgG1 modified to improve binding to one or more Fc gamma receptors; IgG1 modified to minimize effector function (amino acid changes); and IgG1 with altered pH-dependent binding to FcRn. The Fc region can include the entire hinge region, or less than the entire hinge region of the antibody.

In some aspects, IgG2-4 hybrids and IgG4 mutants have reduced binding to FcR which can increase their half-life. Representative IG2-4 hybrids and IgG4 mutants are described, for example, in Angal et al., 1993, Molec. Immunol., 30(1):105-108; Mueller et al., 1997, Mol. Immun., 34(6):441-452; and U.S. Pat. No. 6,982,323; all of which are hereby incorporated by references in their entireties. In some aspects, the IgG1 and/or IgG2 domain can be deleted. For example, Angal et al., Id., describe proteins in which IgG1 and IgG2 domains have serine 241 replaced with a proline. In some aspects, fusion proteins or polypeptides having at least 10, at least 20, at least 30, at least 40, at least 50, at least 60, at least 70, at least 80, at least 90 or at least 100 amino acids are contemplated.

In some aspects, anti-SLO antibodies or polypeptides can be linked to or covalently bind or form a complex with at least one moiety. Such a moiety may be, but is not limited to, one that increases the efficacy of the antibody as a diagnostic or a therapeutic agent. In some aspects, the moiety can be an imaging agent, a toxin, a therapeutic enzyme, an antibiotic, a radio-labeled nucleotide, and the like.

In some aspects, antibodies and polypeptides as described herein may be conjugated to a marker, such as a peptide, to facilitate purification. In some aspects, the marker can be a hexa-histidine peptide, i.e., the hemagglutinin “HA” tag, which corresponds to an epitope derived from the influenza hemagglutinin protein (Wilson, I. A. et al., Cell, 37:767-778 (1984)), or the “flag” tag (Knappik, A. et al., Biotechniques 17(4):754-761 (1994)).

In some aspects, the moiety conjugated to the antibodies and polypeptides as described herein can be an imaging agent that can be detected in an assay. Such imaging agents can be enzymes, prosthetic groups, radiolabels, nonradioactive paramagnetic metal ions, haptens, fluorescent labels, phosphorescent molecules, chemiluminescent molecules, chromophores, luminescent molecules, bioluminescent molecules, photoaffinity molecules, or colored particles or ligands, such as biotin. In some aspects, suitable enzymes include, but are not limited to, horseradish peroxidase, alkaline phosphatase, beta-galactosidase, or acetylcholinesterase; prosthetic group complexes include, but are not limited to, streptavidin/biotin and avidin/biotin; fluorescent materials include, but are not limited to, umbelliferone, fluorescein, fluorescein isothiocyanate, rhodamine, dichlorotriazinylamine fluorescein, dansylchloride or phycoerythrin; luminescent materials include, but are not limited to, luminol; bioluminescent materials include, but are not limited to, luciferase, luciferin, and aequorin; radioactive materials include, but are not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthenium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

The imaging agent can be conjugated to the antibodies or polypeptides described herein either directly or indirectly through an intermediate (such as, for example, a linker known in the art) using techniques known in the art. See, for example, U.S. Pat. No. 4,741,900 which reports on metal ions that can be conjugated to antibodies and other molecules as described herein for use as diagnostics. Some conjugation methods involve the use of a metal chelate complex employing, for example, an organic chelating agent, such as diethylenetriaminepentaacetic acid anhydride (DTPA); ethylenetriaminetetraacetic acid; N-chloro-p-toluenesulfonamide; and/or tetrachloro-3-6α-diphenylglycouril-3, attached to the antibody. Monoclonal antibodies can also be reacted with an enzyme in the presence of a coupling agent such as glutaraldehyde or periodate. Conjugates with fluorescein markers can be prepared in the presence of these coupling agents or by reaction with an isothiocyanate.

In some aspects, the anti-SLO antibodies polypeptides as described herein can be conjugated to a second antibody to form an antibody heteroconjugate, for example, as described in U.S. Pat. No. 4,676,980. Such heteroconjugate antibodies can additionally bind to haptens (e.g., fluorescein), or to cellular markers.

In some aspects, the anti-SLO antibodies or polypeptides described herein can also be attached to solid supports, which can be useful for carrying out immunoassays or purification of the target antigen or of other molecules that are capable of binding to the target antigen that has been immobilized to the support via binding to an antibody or antigen binding fragment as described herein. Such solid supports include, but are not limited to, glass, cellulose, polyacrylamide, nylon, polystyrene, polyvinyl chloride or polypropylene.

### KITS and DIAGNOSTICS

Disclosed herein are kits comprising therapeutic agents and/or other therapeutic and delivery agents. In some aspects, the kits can be used for preparing and/or administering a therapy involving the antibodies or antibody fragments described herein. In some aspects, the kits can be used for preparing and/or administering a therapy involving the fusion polypeptides or conjugates described herein. In some aspects, the kits comprise any of the peptides, antibodies, and antibody fragments described herein. The kits can comprise one or more sealed vials containing any of the pharmaceutical compositions as described herein. The kits can include, for example, at least one anti-SLO antibody, as well as reagents to prepare, formulate, and/or administer one or more anti-SLO antibodies or to perform one or more steps of the described methods. In some aspects, the kits can also comprise a suitable container means, which is a container that will not react with components of the kit, such as an Eppendorf tube, an assay plate, a syringe, a bottle, or a tube. The container may be made from sterilizable materials, such as plastic or glass.

The kits can further include an instruction sheet that outlines the procedural steps of the methods set forth herein, and will follow substantially the same procedures as described herein or are known to those of ordinary skill. The instruction information may be in a computer readable medium containing machine-readable instructions that, when executed using a computer, cause the display of a real or virtual procedure of delivering a pharmaceutically effective amount of the therapeutic agent.

### EXAMPLES

### Example 1: Human Monoclonal Antibodies (huMAbs) to Model Cholesterol-Dependent Cytolysin, Streptolysin O (SLO), from Group A Streptococcus

Severe necrotizing soft tissue infections (NSTIs) which cause exceedingly high morbidity and mortality despite modern antibiotic regimens and intensive care measures. In each case, potent, well-characterized extracellular toxins mediate the rapid destruction of skin, subcutaneous tissues, fascia and muscle, often leading to multiple organ failure and toxic shock. Extensive tissue debridement, often including amputation, is important for source control.

A need for a broad-spectrum anti-toxin therapy that can be applied at the earliest suspicion of NSTI is needed. Such treatment modality would slow the progression of disease while specific pathogens sensitivities are determined, differential diagnosis is made, and surgical intervention is initiated. A broad-spectrum, anti-toxin therapy can then be applied in the emergency setting to slow the progression of disease and improve patient outcomes. As described herein, a fully human, high-titer, toxin-specific passive immunotherapy was generated that can be delivered early to neutralize the major exotoxins of the collective group of principal pathogens causing necrotizing infections. This approach can overcome the existing clinical and laboratory deficiencies to provide a broad-spectrum, clinically relevant treatment for use in the earliest stages of illness.

To this end, a multi-valent, multi-species cocktail of toxin-neutralizing antibodies for clinical use was generated. More specifically, fully human monoclonal antibodies (huMAbs) were developed to model cholesterol-dependent cytolysin, streptolysin O (SLO), from Group A Streptococcus (GAS or Streptococcus pyogenes).

METHODS. Antigen expression and modification. Oxygen stable SLO (herein SLO wt; GenBank accession P0DF96 (2)) was cloned into the pTrcHiS vector (Lamb C L, et al. 2019. Enrichment of Antigen-Specific Class-Switched B Cells from Individuals Naturally Immunized by Infection with Group A Streptococcus. mSphere 4; and Kehoe M A, et al. 1987. Infect Immun 55:3228-32). To generate a molecule that could be isolated with high specificity and maintain the antigenic structure of SLO, an N-terminal domain (NTD) was constructed with the following modifications, starting with the amine terminus: His-tag (His6) for Ni-affinity purification, mono-biotinylation BirA site, and a flexible linker consisting of four glycine and one serine residues. Mutations in the cholesterol recognition motif equivalent to T564G and L565G (herein SLOm (Tweten R K, et al. 2015. Annu Rev Microbiol 69:323-40), were generated from SLOwt using Q5 Site-Directed Mutagenesis Kit mutagenesis (New England Bio Labs, Ipswich, MA) and verified by DNA sequencing analysis performed by Molecular Cloning Laboratories (MCLAB, San Francisco, CA). The expression and purification of recombinant His-tagged SLO and its derivatives from Escherichia coli (Rosetta DE3 pLysS competent cells) were carried out as described (Shepard L A, et al. 1998. Biochemistry 37:14563-74) with modifications. Briefly, crude protein lysate was loaded onto a HisTrapTMHP (GE Healthcare, Chicago, IL) and washed with 10 column volumes of buffer A (100 mM Tris-HCl pH 7.5, 500 mM NaCl, 0.02% NaN3) plus 4% buffer B (100 mM Tris-HCl pH 7.5, 500 mM NaCl, 1 M imidazole, 0.02% NaN3). Recombinant SLO was eluted from the column with 30 column volumes of 4% to 100% buffer B gradient. The eluted protein was buffer exchanged into freezing buffer (50 mM Hepes pH 7.5, 100 mM NaCl, 1 mM EDTA, 1 mM TCEP, 0.02% NaN3). The toxin was then stored in 10% (v/v) glycerol at −80° C. until used. Mono-biotinylation was carried out endogenously by Rosetta DE3 pLysS competent cells and verified by anti-biotin ELISA.

Antigen specific B-cell enrichment. Healthy participants who had previously suffered a mild to severe GAS infection were included in the study. Heparinized blood was drawn from a peripheral vein, a small sample was collected from the donors for determination of ASO titers as an indicator of natural immunization status. PBMCs were isolated by density gradient centrifugation with Ficoll-PaquePlus (GE Healthcare). Briefly, blood was diluted three times the initial volume with PBS and layered over 20 mL of Ficoll-Paque Plus. Following centrifugation at 400×g for 40 minutes, the mononuclear cell layer was removed and diluted 1:1 with PBS. Cells were washed and counted using trypan blue exclusion.

For the isolation of B-cells, roughly 1×108 PBMCs were resuspended in rinsing solution (MACS, Miltenyi Biotec, Bergisch Gladbach, Germany). Non-B-cells (Pan B-cell Kit, Miltenyi Biotec) and B-cells expressing IgM (Anti-IgM, Miltenyi Biotec) were labeled by immuno-superparamagnetic microbeads and depleted following manufacture protocols. Briefly, 500 μL of labeled cells were bound to an LD column (Miltenyi Biotec) in the VarioMACS™ Separator (Miltenyi Biotec). B-cells in the flow-through were collected (herein “pre-enriched”) and PBMCs captured in the column were disposed. Pre-enriched B-cells were counted using trypan blue exclusion.

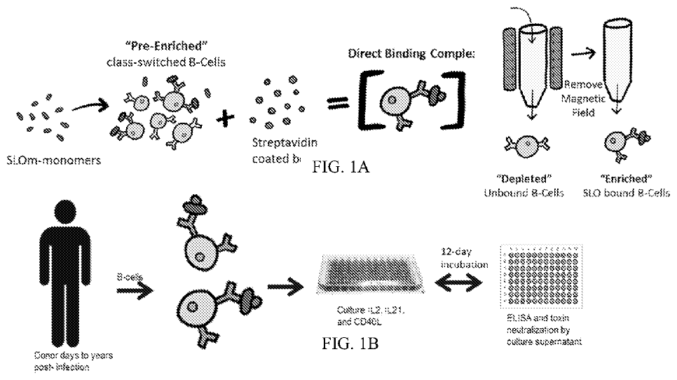

As illustrated in FIG. 1A (Lamb C L, et al. 2019. Enrichment of Antigen-Specific Class-Switched B Cells from Individuals Naturally Immunized by Infection with Group A Streptococcus. mSphere 4), the antigen specific cell isolations were performed using magnetic-activated cell sorting (MACS) and recombinant SLO mutated at cholesterol binding residues (SLOm). Pre-enriched B-cells were incubated with 6.25 nM mono-biotinylated SLOm on ice for 30 minutes, then incubated with streptavidin coated superparamagnetic microbeads (Miltenyi Biotec) following manufacturer protocol. Following the labeling method, B-cells were passed over an MS column (Miltenyi Biotec) in a VarioMACS™ Separator for positive selection of the SLO-specific B-cells (herein “enriched”). The enriched cell fraction was eluted off the column after removal from the magnetic field.

Propagation and identification of antigen specific B-cells. As illustrated in FIG. 1B, human B-cells enriched were cultured in 384-well plates in Iscove's Modified Dulbecco's Medium (IMDM, Gibco, Fisher Scientific) supplemented with 10% HI FBS (Gibco), 2:1000 MycoZap (Lonza, Alpharetta, GA), 10,000 U/mL IL2 (Gibco), and 100 μg/mL IL21 (BioLegend), and irradiated 3T3-msCD40L feeder cells (NIH: Cat #12535 3T3-msCD40L cells; Huang J, et al. 2013. Nat Protoc 8:1907-1915). B-cells were plated at 4 cells/well and incubated at 37° C. in 5% CO₂. After 12 days, plates were centrifuged (400×g) supernatant was transferred into clean 384-well plates labeled with the collection date, number of days incubated and the sample ID. Twenty μL of lysis buffer [1×DPBS, without calcium and magnesium (Sigma), 10 mM DTT and Nuclease-free water (Qiagen) (von Boehmer L, et al. 2016. Nat Protoc 11:1908-1923)] was added to the B-cells. Both cells and supernatant were stored at −80° C. for downstream analysis.

From B-cell culture supernatant, IgG and SLO specificity was determined by enzyme-linked immunosorbent assay (ELISA). Briefly, to quantify IgG, high-binding 96-well plates were coated with 0.5 pg/mL unconjugated goat anti-human IgG Fc capture antibody (Invitrogen, Thermo Fisher Scientific). To qualify antibodies binding SLO, streptavidin coated 96-well plates were incubated with biotinylated SLOwt at 37° C. for 1 hour. Cell culture supernatants were diluted in sample buffer (PBS plus 5% non-fat dry milk) and incubated on plates for 1 hour at 37° C. Horseradish peroxidase conjugated goat anti-human IgG antibody (Thermo Scientific) was used for detection and 1-Step Ultra TMB-ELISA Substrate solution (Thermo Scientific) was used to detect horseradish peroxidase activity according to manufacturer protocol. Reaction was stopped with the addition of 2M sulfuric acid and absorbance was read at 450 nm.

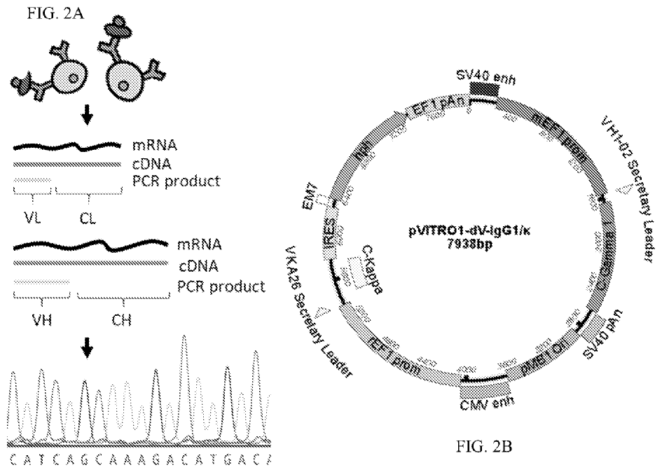

Human antibody cloning. FIG. 2 shows the generation of cDNA (von Boehmer L, et al. 2016. Nat Protoc 11:1908-1923). Briefly, 1.65 μL of 40 U/μL RNaseOUT (Invitrogen) were added to frozen plates or wells containing anti-SLO IgG producing B-cells. Thaw plates on ice then centrifuge at 400×g for 30 seconds at 4° C. Remove 4 μl of lysed B-cells into PCR tubes containing 7 μL of reverse transcription mix I [0.6% IGEPAL (Sigma), 18.3 ng/μL random primers (Invitrogen, Cat. No. 48190-011), 0.8 U/μL RNaseOUT, in nuclease free water]. Incubate samples for 5 minutes at 65° C. Place plate on ice for 5 minutes and add 7 μL of reverse transcription mix II [2.3× SuperScript IV buffer (ThermoFisher), 14 mM DTT (Sigma), 1.8 mM dNTPs, 0.6 U/μL RNaseOut, 7.1 U/μl SuperScript IV RT (ThermoFisher)]. Centrifuge samples at 400×g for 1 minute at 4° C. Place samples in thermocycler run program: 42° C. for 10 min, 25° C. for 10 min, 50° C. for 10 min, 94° C. for 5 min, hold at 4° C.

To amplify cDNA (PCR product, FIG. 2A), add 4 μL of reverse transcription reaction to 21 μL of 1st PCR mix [1×Q5 reaction buffer (NEB), 200 pM dNTPs, 0.5 pM forward primers, 0.5 pM reverse primer (Table 4; (Lin Z, et al. 2014. Nat Protoc 9:1563-1577)), 0.02 U/ul Q5 HiFi DNA polymerase (NEB), 1×Q5 High GC enhancer (NEB), nuclease free water to 25 μl/reaction]. Centrifuge samples for 1 minute at 4° C. Place samples in thermocycler run program: 1×98° C. 30 seconds; 35×98° C. 10 minutes, 65° C. 30 seconds, 72° C. 40 seconds; 1×72° C. 120 seconds, hold at 4° C. Analyze samples by 2% agarose gel [1×TAE (ThermoFisher), 2% certified PCR grade agarose (BioRad), 1×SYBR safe (ThermoFisher)] electrophorese. Samples with heavy and light chain products from matched wells undergo 2nd round nested PCR reaction. Add 1 ul of 1st round PCR reaction to 9 μl of 2nd round PCR mix [1× Phusion Flash PCR master mix (Invitrogen), 0.5 pM Forward primers, 0.5 pM reverse primer (Table 5; (Lin Z, et al. 2014. Nat Protoc 9:1563-1577)), nuclease free water to volume]. Centrifuge samples at 400×g for 1 minute at 4° C. Place samples in thermocycler run program: 1×98° C. 30 seconds; 30×98° C. 10 seconds, 56° C. 15 seconds, 72° C. 10 seconds; 1×72° C. 60 seconds; hold at 4° C. Analyze samples in 2% agarose gel electrophorese. Sequence hits using the Table 6 reverse primers.

TABLE 4
First round of PCR Primers

| Primer | Sequence | T′A | Reverse Complement |
|---|---|---|---|
| IGVH1a | CAGGTGCAGCTGGTGCAGTCTGGGGC | 72 | GCCCCAGACTGCACCAGCTGCACCTG |
| | (SEQ ID NO: 28) | | (SEQ ID NO: 29) |
| IGVH1b | CAGGTCCAGCTGGTGCAGTCTGGGGC | | GCCCCAGACTGCACCAGCTGGACCTG |
| | (SEQ ID NO: 30) | | (SEQ ID NO: 31) |
| IGVH2 | CAGGTCACCTTGAAGGAGTCTGGTCC | | GGACCAGACTCCTTCAAGGTGACCTG |
| | (SEQ ID NO: 32) | | (SEQ ID NO: 33) |
| IGVH3 | GAGGTGCAGCTGGTGGAGTCTGGGGG | | CCCCCAGACTCCACCAGCTGCACCTC |
| | (SEQ ID NO: 34) | | (SEQ ID NO: 35) |
| IGVH4 | CAGGTGCAGCTGCAGGAGTCGGGCCC | | GGGCCCGACTCCTGCAGCTGCACCTG |
| | (SEQ ID NO: 36) | | (SEQ ID NO: 37) |
| IGVH5 | GAGGTGCAGCTGGTGCAGTCTGG | | CCAGACTGCACCAGCTGCACCTC |
| | (SEQ ID NO: 38) | | (SEQ ID NO: 39) |
| IGVH6 | CAGGTACAGCTGCAGCAGTCAGGTCC | | GGACCTGACTGCTGCAGCTGTACCTG |
| | (SEQ ID NO: 40) | | (SEQ ID NO: 41) |
| IGVH7 | CAGGTGCAGCTGGTGCAATCTGG | | CCAGATTGCACCAGCTGCACCTG |
| | (SEQ ID NO: 42) | | (SEQ ID NO: 43) |
| HC301.5 | GCAGCCCAGGGCSGCTGTGC | | GCACAGCSGCCCTGGGCTGC |
| constant | (SEQ ID NO: 44) | | (SEQ ID NO: 45) |
| IGKV1 | GHCATCCRGWTGACCCAGTCTC | 68 | GAGACTGGGTCAWCYGGATGDC |
| | (SEQ ID NO: 46) | | (SEQ ID NO: 47) |
| IGKV2 | GATRTTGTGATGACYCAGWCTC | 58 | GAGWCTGRGTCATCACAAYATC |
| | (SEQ ID NO: 48) | | (SEQ ID NO: 49) |
| IGKV3 | GAAATWGTRWTGACRCAGTCTC | 58 | GAGACTGYGTCAWYACWATTTC |
| | (SEQ ID NO: 50) | | (SEQ ID NO: 51) |
| IGKV4 | GACATCGTGATGACCCAGTCTCC | 68 | GGAGACTGGGTCATCACGATGTC |
| | (SEQ ID NO: 52) | | (SEQ ID NO: 53) |
| IGKV5 | GAAACGACACTCACGCAGTCTC | 68 | GAGACTGCGTGAGTGTCGTTTC |
| | (SEQ ID NO: 54) | | (SEQ ID NO: 55) |
| IGKV6 | GAWRTTGTGMTGACWCAGTCTC | 58 | GAGACTGWGTCAKCACAAYWTC |
| | (SEQ ID NO: 56) | | (SEQ ID NO: 57) |
| Kappa102 | GCACACAACAGAGGCAGTTCCAG | 68/58 | (SEQ ID NO: 59) |
| constant | (SEQ ID NO: 58) | | CTGGAACTGCCTCTGTTGTGTGC |
| IGL V1 | CAGTCTGTGYTGACKCAGCCRCCCTC | 65 | GAGGGYGGCTGMGTCARCACAGACTG |
| | (SEQ ID NO: 60) | | (SEQ ID NO: 61) |
| IGL V2 | CAGTCTGCCCTGACTCAGCCT | | AGGCTGAGTCAGGGCAGACTG |
| | (SEQ ID NO: 62) | | (SEQ ID NO: 63) |
| IGL V3 | TCCTATGAGCTGACWCAGSHVCCCKC | | GMGGGBDSCTGWGTCAGCTCATAGGA |
| | (SEQ ID NO: 64) | | (SEQ ID NO: 65) |
| IGL V4 | CAGCCTGTGCTGACTCARTCVCCCTC | | GAGGGBGAYTGAGTCAGCACAGGCTG |
| | (SEQ ID NO: 66) | | (SEQ ID NO: 67) |
| IGL V5 | CAGCCTGTGCTGACTCAGCCAACTTC | | GAAGTTGGCTGAGTCAGCACAGGCTG |
| | (SEQ ID NO: 68) | | (SEQ ID NO: 69) |
| IGL V6 | AATTTTATGCTGACTCAGCCCCAC | | GTGGGGCTGAGTCAGCATAAAATT |
| | (SEQ ID NO: 70) | | (SEQ ID NO: 71) |
| IGLV7 | CAGGCTGTGGTGACTCAGGAGCCC | | GGGCTCCTGAGTCACCACAGCCTG |
| | (SEQ ID NO: 72) | | (SEQ ID NO: 73) |
| IGL V8 | CAGACTGTGGTGACCCAGGAGCC | | GGCTCCTGGGTCACCACAGTCTG |
| | (SEQ ID NO: 74) | | (SEQ ID NO: 75) |
| IGL V9 | CAGCCTGTGCTGACTCAGCCACC | | GGTGGCTGAGTCAGCACAGGCTG |
| | (SEQ ID NO: 76) | | (SEQ ID NO: 77) |
| Lambda202 | \|CTTGRAGCTCCTCAGAGGAG | | CTCCTCTGAGGAGCTYCAAG |
| constant | (SEQ ID NO: 78) | | (SEQ ID NO: 79) |

T′A is the annealing temperature in ° C.

TABLE 5
Second round of PCR Primers

| Primer | Sequence | T′A | Reverse Complement |
|---|---|---|---|
| sVH1ar | CCGCCACAGGCGCGCACTCCCAGG | 63 | CTGCACCAGCTGCACCTG |
| | TGCAGCTGGTGCAG | | (SEQ ID NO: 81) |
| | (SEQ ID NO: 80) | | |
| sVH2r | CCGCCACAGGCGCGCACTCCCAGA | | CAGACTCCTTCAAGGTGATCTG |
| | TCACCTTGAAGGAGTCTG | | (SEQ ID NO: 83) |
| | (SEQ ID NO: 82) | | |
| SVH3vvr | CCGCCACAGGCGCGCACTCCCAGG | | CTCCACCAGCTGCACCTG |
| | TGCAGCTGGTGGAG | | (SEQ ID NO: 85) |
| | (SEQ ID NO: 84) | | |
| sVHg1r | CCGCCACAGGCGCGCACTCCGAGG | | CTCCACCAGCTGCACCTC |
| | TGCAGCTGGTGGAG | | (SEQ ID NO: 87) |
| | (SEQ ID NO: 86) | | |
| sVH4r | CCGCCACAGGCGCGCACTCCCAGG | | CTCCTGCAGCTGCACCTG |
| | TGCAGCTGCAGGAG | | (SEQ ID NO: 89) |
| | (SEQ ID NO: 88) | | |
| SVH5r | CCGCCACAGGCGCGCACTCCGAGG | | CTGCACCAGCTGCACCTC |
| | TGCAGCTGGTGCAG | | (SEQ ID NO: 91) |
| | (SEQ ID NO: 90) | | |
| sVH6r | CCGCCACAGGCGCGCACTCCCAGG | | CTGCTGCAGCTGTACCTG |
| | TACAGCTGCAGCAG | | (SEQ ID NO: 93) |
| | (SEQ ID NO: 92) | | |
| SVH7r | CCGCCACAGGCGCGCACTCCCAGG | | GATTGCACCAGCTGCACCTG |
| | TGCAGCTGGTGCAATC | | (SEQ ID NO: 95) |
| | (SEQ ID NO: 94) | | |
| Hinsert_ | GGGAAGTAGTCCTTGACCAGGCAG | | CTCCAAGAGCACCTCTGGGGG |
| REV | CCCAGGGCCGCTGTGCCCCCAGAG | | CACAGCGGCCCTGGGCTGCCTG |
| | GTGCTCTTGGAG | | GTCAAGGACTACTTCCC |
| | (SEQ ID NO: 96) | | (SEQ ID NO: 97) |
| dsVK1r* | GGGTTCCAGCTAGCCGCGGTGACA | 56 | CTGGGTCATCTGGATGTC |
| | TCCAGATGACCCAG | | (SEQ ID NO: 99) |
| | (SEQ ID NO: 98) | | |
| dsVK2r* | GGGTTCCAGCTAGCCGCGGTGATA | | GACTGAGTCATCACAATATC |
| | TTGTGATGACTCAGTC | | (SEQ ID NO: 101) |
| | (SEQ ID NO: 100) | | |
| dsVK3r* | GGGTTCCAGCTAGCCGCGGTGAAA | | CTGTGTCAACACAATTTC |
| | TTGTGTTGACACAG | | (SEQ ID NO: 103) |
| | (SEQ ID NO: 102) | | |
| dsVK4r* | GGGTTCCAGCTAGCCGCGGTGACA | | CTGGGTCATCACGATGTC |
| | TCGTGATGACCCAG | | (SEQ ID NO: 105) |
| | (SEQ ID NO: 104) | | |
| dsVK5r* | GGGTTCCAGCTAGCCGCGGTGAAA | | CTGCGTGAGTGTCGTTTC |
| | CGACACTCACGCAG | | (SEQ ID NO: 107) |
| | (SEQ ID NO: 106) | | |
| dsVK6r* | GGGTTCCAGCTAGCCGCGGTGAAA | | CTGAGTCAGCACAATTTC |
| | TTGTGCTGACTCAG | | (SEQ ID NO: 109) |
| | (SEQ ID NO: 108) | | |
| Kinsert_ | CTGGGATAGAAGTTATTCAGCAGG | | GAGCAGTTGAAATCTGGAACT |
| REV | CACACAACAGAGGCAGTTCCAGAT | | GCCTCTGTTGTGTGC |
| | TTCAACTGCTC | | (SEQ ID NO: 111) |
| | (SEQ ID NO: 110) | | |
| dsVL1* | ATGGATCCGGAGTCGACTCTCAGT | 60 | CTGMGTCARCACAGACTG |
| | CTGTGYTGACKCAG | | (SEQ ID NO: 113) |
| | (SEQ ID NO: 112) | | |
| dsVL2* | ATGGATCCGGAGTCGACTCTCAGT | | CTGAGTCAGGGCAGACTG |
| | CTGCCCTGACTCAG | | (SEQ ID NO: 115) |
| | (SEQ ID NO: 114) | | |
| dsVL3* | ATGGATCCGGAGTCGACTCTTCCTA | | CTGWGTCAGCTCATAGGA |
| | TGAGCTGACWCAG | | (SEQ ID NO: 117) |
| | (SEQ ID NO: 116) | | |
| dsVL4* | ATGGATCCGGAGTCGACTCTCAGC | | GAYTGAGTCAGCACAGGCTG |
| | CTGTGCTGACTCARTC | | (SEQ ID NO: 119) |
| | (SEQ ID NO: 118) | | |
| dsVL5* | ATGGATCCGGAGTCGACTCTCAGC | | CTGAGTCAGCACAGGCTG |
| | CTGTGCTGACTCAG | | (SEQ ID NO: 121) |
| | (SEQ ID NO: 120) | | |
| dsVL6* | ATGGATCCGGAGTCGACTCTAATTT | | GGCTGAGTCAGCATAAAATT |
| | TATGCTGACTCAGCC (SEQ ID | | (SEQ ID NO: 123) |
| | NO: 122) | | |
| dsVL7* | ATGGATCCGGAGTCGACTCTCAGG | | CTGAGTCACCACAGCCTG |
| | CTGTGGTGACTCAG | | (SEQ ID NO: 125) |
| | (SEQ ID NO: 124) | | |
| dsVL8* | ATGGATCCGGAGTCGACTCTCAGA | | CTGGGTCACCACAGTCTG |
| | CTGTGGTGACCCAG | | (SEQ ID NO: 127) |
| | (SEQ ID NO: 126) | | |
| dsVL9* | ATGGATCCGGAGTCGACTCTCAGC | | CTGAGTCAGCACAGGCTG |
| | CTGTGCTGACTCAG | | (SEQ ID NO: 129) |
| | (SEQ ID NO: 128) | | |
| Linsert_ | CACCAGTGTGGCCTTGTTGGCTTGA | | CGGTCACTCTGTTCCCGCCCTC |
| REV | AGCTCCTCAGAGGAGGGCGGGAAC | | CTCTGAGGAGCTTCAAG |
| | AGAGTGACCG | | (SEQ ID NO: 131) |
| | (SEQ ID NO: 130) | | |

T′A is the annealing temperature in ° C.

TABLE 6
Primers to linearize plasmid

| Vector | Primer | Sequence | T′A | Reverse Complement | Fragment |
|---|---|---|---|---|---|
| IgGK | Kfwd | CTGCTGAATAACTTCTATCCCAGA | 72 | CACTGTACTTTGGCCTCTCTGGG | A |
| | | GAGGCCAAAGTACAGTG | | ATAGAAGTTATTCAGCAG | |
| | | (SEQ ID NO: 132) | | (SEQ ID NO: 133) | |
| | Hrev | GGAGTGCGCGCCTGTGGCGGCCG | | GATCCTCTTCTTGGTGGCGGCC | |
| | | CCACCAAGAAGAGGATC | | GCCACAGGCGCGCACTCC | |
| | | (SEQ ID NO: 134) | | (SEQ ID NO: 135) | |
| | Hfwd | CTGGTCAAGGACTACTTCCCCGAA | 72 | ACGACACCGTCACCGGTTCGGG | B |
| | | CCGGTGACGGTGTCGT | | GAAGTAGTCCTTGACCAG | |
| | | (SEQ ID NO: 136) | | (SEQ ID NO: 137) | |
| | Krev | ACCGCGGCTAGCTGGAACCCAGA | | CATTGGGTTTCTGCTGCTCTGGG | |
| | | GCAGCAGAAACCCAATG | | TTCCAGCTAGCCGCGGT | |
| | | (SEQ ID NO: 138) | | (SEQ ID NO: 139) | |
| IgGA | Lfwd | CCAACAAGGCCACACTGGTGTGTC | 72 | TAGAAGTCACTTATGAGACACA | A |
| | | TCATAAGTGACTTCTA | | CCAGTGTGGCCTTGTTGG | |
| | | (SEQ ID NO: 140) | | (SEQ ID NO: 141) | |
| | Hrev | GGAGTGCGCGCCTGTGGCGGCCG | | GATCCTCTTCTTGGTGGCGGCC | |
| | | CCACCAAGAAGAGGATC | | GCCACAGGCGCGCACTCC | |
| | | (SEQ ID NO: 142) | | (SEQ ID NO: 143) | |
| | Hfwd | CTGGTCAAGGACTACTTCCCCGAA | 72 | ACGACACCGTCACCGGTTCGGG | B |
| | | CCGGTGACGGTGTCGT | | GAAGTAGTCCTTGACCAG | |
| | | (SEQ ID NO: 144) | | (SEQ ID NO: 145) | |
| | Lrev | AGAGTCGACTCCGGATCCATAAGC | | TCTCCTCGGACTCCTTGCTTATG | |
| | | AAGGAGTCCGAGGAGA | | GATCCGGAGTCGACTCT | |
| | | (SEQ ID NO: 146) | | (SEQ ID NO: 147) | |

T′A is the annealing temperature in ° C.

For the linearization of vector (pVITRO human IgG1κ or human IgG1λ (Addgene) (Dodev T S, et al. 2014. Sci Rep 4:5885)), in separate PCR tubes, add ˜1 ng of pVITRO to 19 μl of PCR mix [1× Phusion flash PCR master mix, 0.5 μM forward primer, 0.5 μM reverse primer] (Table 6). Place samples in thermocycler run program: 1×98° C. 30 seconds; 30×98° C. 10 seconds, 72° C. 15 seconds, 72° C. 135 seconds; 1×72° C. 60 seconds; 4° C. hold. After PCR add 1 μl DpnI to each sample and incubate at 37° C. for 30 minutes. Heat inactivate DpnI at 80° C. for 20 minutes. Analyze 4 μL of linearized plasmid by 1% agarose gel electrophorese. One linearized vector product should appear in the gel.

Next, VH and VL PCR products were inserted into plasmid according to manufactures protocol [NEBuilder HiFi DNA assembly cloning kit (NEB)]. Briefly, VH and VL fragments, herein inserts, should be purified (PCR clean up Qiagen). In a PCR tube, combine 0.01-0.04 pmol of linearized vectors with 0.02-0.2 pmol inserts, NEBuilder DNA assembly master mix and nuclease free water to 20 μL. Incubate at 50° C. for 4 hours. After assembly, transform cells into DH5a (ThermoFisher) following manufactures protocol. Minprep (Qiagen) plasmid for sequencing of the entire plasmid.

Production of recombinant antibodies. Human antibodies were expressed in human embryonic kidney cells (HEK, Expi293F, ThermoFisher) according to manufactures protocol with minor modifications. Briefly, in 29 mL Expi293 expression medium HEKs were grown in 125 mL Erlenmeyer shake flasks and incubated at 37° C. with 8% CO₂ shaking at 125 rpm. Cells were split at ˜3-5×106 cells/mL (˜every 3-4 days) and seeded at 3-5×105 cells/mL. After at least 4 passages, 7.5×10⁷ cells in a volume of 25.5 mL of expression medium were transfected using the ExpiFectamine 293 transfection kit according to manufactures protocol. Briefly, 2.7 μL of ExpiFectamine 293 reagent per 1 μg of plasmid DNA was diluted in Opti-MEM to a 1.5 mL final volume. Thirty pg of plasmid DNA was diluted into 1.5 mL of Opti-MEM (ThermoFisher). Both DNA and lipids incubated separately for 5 minutes at room temperature, then were combined and incubated for 20 minutes at room temperature, then added dropwise to the cells. Transfection reactions were incubated at 37° C. in 8% CO₂ while shaking at 125 rpm. About 17 hours post transfection, add 150 ul of ExpiFectamine 293 Transfection Enhancer 1 and 1.5 ml of ExpiFectamine 293 Transfection Enhancer 2 to the flask. The final volume should be about 30 ml per well. After one week, collect cell culture supernatant by centrifugation 3000×g for 10 minutes at 4° C., two times.

Western blot. To confirm antibody binding to SLO, non-biotinylated and biotinylated WT SLO, along with BSA, was diluted 3:1 in 4× Laemmlli Sample Buffer (BioRad) with final 355 mM BME as the reducing agent. Samples were incubated at 95° C. for 5 mins before being loaded onto a 4-15% Mini-Protean TGX Precast Protein Gel (BioRad) with 1× Tris/Glycine/SDS Buffer (BioRad). The gel ran for about 30 mins at 200 V. Once finished, the gel was transferred to PVDF using iBlot (Invitrogen).

For the Western, the PVDF membrane was blocked with SuperBlock (Thermo) in the SNAP i.d. Protein Detection System (Millipore) per the manufacturer protocol. The produced recombinant antibody was used as the primary antibody (1:100) and Goat anti-human IgG HRP for the secondary (1:2,000, Thermo). The Western was developed with 1-Step Ultra TMB-Blotting Solution (Thermo).

Neutralization of SLO toxicity and membrane binding. For neutralization of red blood cell lysis (RBC), 4-8 HU of SLOwt, diluted in PBS, was added to 25 μL of PBS or antibody (two-fold dilutions from 800 to 0.1 nM) and incubated at 37° C. for 15 minutes. Following incubation, 50 μL of a 2% suspension of washed RBCs (Hardy Diagnostics) was added to each well and plate was incubated for 30 minutes at 37° C. Non-lysed erythrocytes were pelleted by centrifugation at 1000×g for 10 min supernatant was transferred to clean 96-well palate and hemolysis was quantified by absorbance (A550). Percent hemolysis was determined by normalization, where 100% and 0% hemolysis were completed by treating RBCs with 25 μL of water or PBS, respectively, in place of SLO+/−antibody.

For cytotoxicity studies, lung alveolar epithelial cells (A549s) were treated with 100 nM SLOwt in the presence of various concentrations of antibody (two fold dilutions from 1000 to 0.001 nM) and Zombie Green™ (cell viability stain; BioLegend, San Diego, CA) for 15 minutes at room temperature in a final volume of 100 μL. Cell viability was determined by flow cytometry (Accuri C6 Plus, BD, San Jose, CA). The antibody concentration required for 50% inhibition (IC50) was determined by nonlinear regression: Y=Bottom+(Top−Bottom)/(1+(X/IC50)), where Top and Bottom are the plateaus.

For analysis of membrane binding, 1×106 PBS washed cells (RBCs or A549s) were treated with 100 nM SLOm and various concentration of antibody (two fold dilutions from 1000 to 0.001 nM) for 60 minutes on ice in a final volume of 100 μL. SLOm was detected fluorescently labeled with 1:100 dilution of mouse anti-SLO monoclonal primary (Abcam, Cambridge, United Kingdom) in PBS and 1:80 dilution of rat anti-mouse IgG-PE secondary in PBS (BioLegend). Samples were brought to a final volume of 500 μL with ice-cold PBS plus 0.1% heat inactivated (HI) FBS. The population of SLO bound to cells was determined by flow cytometry (BD Accuri C6 Plus), gating on both forward scatter and fluorescence. Specific binding was analyzed by nonlinear regression.

RESULTS. From a single naturally immunized donor, about 15% of wells were positive for IgG, about 10% of which were SLO specific. Of those, about 70% were successfully reverse transcribed and PCR amplified for a final of 8 paired human antibody sequences. Human antibodies were cloned into a dual heavy and light chain antibody expression vector containing the constant domains of IgG1 and κ or λ light chains (pVITRO1). Light chain variable regions were cloned on to their respective κ or λ constant domains base on sequence analysis (IMGT.org and IgBlast NCBInih.gov). Of the 8 antibody sequences, six were predicted to product a productive product using IMGT.org and those were successfully subcloned into the expression vector. After transfection 5 antibodies were soluble and/or expressed at useable concentrations (10-100 mg/L).

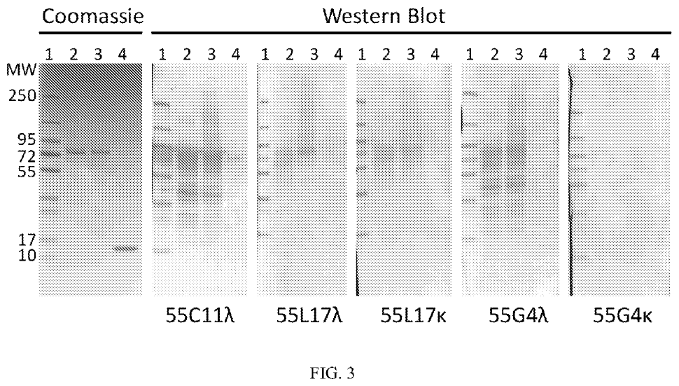

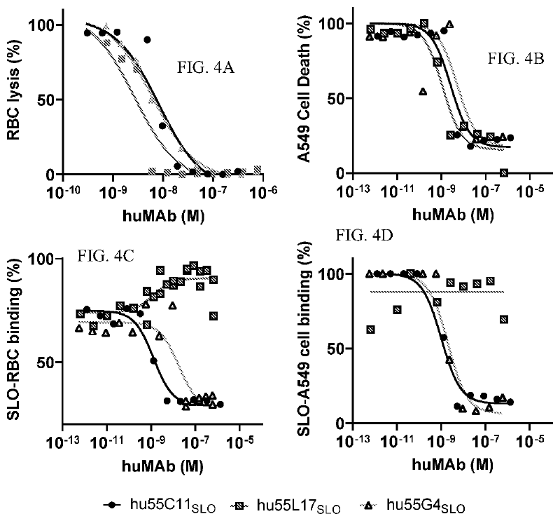

Western blots confirm that four of five antibodies are specific to SLO and do not bind other molecules present during B-cell enrichment (biotin and streptavidin; FIG. 3). All huMAbs bind SLO with pM affinity as determined by ELISA. The ability to neutralize cytotoxic effects of SLOwt was evaluated by the ability to protect RBCs from SLOwt-induced hemolysis and epithelial cell (A549) death. Results show that the antibodies were neutralizing with an average IC50 of 5.6 nM, a concentration similar to other toxin neutralizing antibodies with therapeutic potential (see, FIG. 4, and Table 7).

Next, antibodies were evaluated for the mechanism by which they inhibit SLOwt-induced lysis. SLOwt pore formation is a multi-step process in which SLO binds host cell membrane cholesterol and oligomerizes into a circular formation. After oligomerization, large conformational changes form a pre-pore β-barrel, followed by another conformation change for insertion of the SLO-oligomer into the host cell membrane (Tweten R K, et al. 2015. Annu Rev Microbiol 69:323-40). Preventing any steps in pore formation results in neutralization of SLO in a standard RBC hemolysis assay. Thus, the anti-SLO neutralizing antibodies were evaluated for their ability to block SLOwt from binding to RBCs and epithelial cell membranes. Interestingly, two antibodies (55C11 and 55G4) were able to block SLOwt-membrane binding, while 55L17 had no effect on membrane biding. The binding epitopes of these huMAbs are unknown, but the different mechanisms of SLO neutralization suggests that the epitopes are different. Additionally, the complementarity determining region (CDR) sequences of each of these huMAbs is different for each anti-SLO antibody.

TABLE 7
Characteristics of anti-SLO human monoclonal antibodies.

| MAb_{SLO} | CDR* | | aHU** | aCTX** | aMB_{RBC}** | aMB_{EP}** |
|---|---|---|---|---|---|---|
| name | VH | Kd (M) | IC50 (M)^{†} | IC50 (M) | IC50 (M) | IC50 (M) |
| hu55C11λ | 8.8.21 | 3.8E−11 | 4.4E−9 | 2.6E−9 | 1.3E−9 | 1.1E−9 |
| hu55L17κ | 8.8.23 | 7.8E−11 | 2.7E−9 | 1.1E−9 | NAF | NAF |
| hu55G4λ | 8.8.20 | 4.6E−11 | 6.3E−9 | 5.1E−9 | 7.9E−9 | 2.0E−9 |

*Variable heavy (VH) complementarity-determining region (CDR) 1, 2 and 3 amino acids numbers
**Anti-hemolytic units (aHU); anti-cytotoxicity on epithelial cells (aCTX); anti-membrane binding on RBCs (aMB_{RBC}) or epithelial cells (aMB_{EP}).
^{†}Concentration of 50% inhibition (IC50) in molarity (M).
NAF, not able to fit because hu55L17 does not prevent SLO binding.

What is claimed is:

1. An isolated antibody or antibody fragment thereof comprising:
a) a light chain variable region, wherein the light chain variable region comprises a complementarity determining region light chain 1 (CDRL1) amino acid sequence of SEQ ID NO: 1 or SEQ ID NO: 1 having at least 1 conservative substitution; a determining region light chain 2 (CDRL2) amino acid sequence of SEQ ID NO: 2 or SEQ ID NO: 2 having at least 1 conservative substitution; and a determining region light chain 3 (CDRL3) amino acid sequence of SEQ ID NO: 3 or SEQ ID NO: 3 having at least 1 conservative substitution; and
b) a heavy chain variable region, wherein the heavy chain variable region comprises a complementarity determining region heavy chain 1 (CDRH1) amino acid sequence of SEQ ID NO: 4 or SEQ ID NO: 4 having at least 1 conservative substitution; a complementarity determining region heavy chain 2 (CDRH2) amino acid sequence of SEQ ID NO: 5 or SEQ ID NO: 5 having at least 1 conservative substitution; and a complementarity determining region heavy chain 3 (CDRH3) amino acid sequence of SEQ ID NO: 6 or SEQ ID NO: 6 having at least 1 conservative substitution.

2. The isolated antibody or antibody fragment thereof of claim 1, wherein the light chain variable region comprises the CDRL1 amino acid sequence of SEQ ID NO: 1; the CDRL2 amino acid sequence of SEQ ID NO: 2; and the CDRL3 amino acid sequence of SEQ ID NO: 3; and wherein the heavy chain variable region comprises the CDRH1 amino acid sequence of SEQ ID NO: 4; the CDRH2 amino acid sequence of SEQ ID NO: 5; and the CDRH3 amino acid sequence of SEQ ID NO: 6.

3. The isolated antibody or antibody fragment thereof of claim 1 or 2, wherein the light chain variable region amino acid has the sequence of SEQ ID NO: 7.

4. The isolated antibody or antibody fragment thereof of claim 1 or 2, wherein the heavy chain variable region amino acid comprises the sequence of SEQ ID NO: 8.

5. An isolated antibody or antibody fragment thereof comprising a light chain variable region amino acid sequence of SEQ ID NO: 7 and a heavy chain variable region amino acid sequence of SEQ ID NO: 8.

6. The isolated antibody or antibody fragment thereof of claim 1, wherein the light chain variable region has an amino acid sequence that is at least 90% identical to amino acid sequence of SEQ ID NO: 7.

7. The isolated antibody or antibody fragment thereof of claim 1, wherein the heavy chain variable region has an amino acid sequence that is at least 90% identical to amino acid sequence of SEQ ID NO: 8.

8. An isolated antibody or antibody fragment thereof comprising:
a) a light chain variable region, wherein the light chain variable region comprises a complementarity determining region light chain 1 (CDRL1) amino acid sequence of SEQ ID NO: 9 or SEQ ID NO: 9 having at least 1 conservative substitution; a determining region light chain 2 (CDRL2) amino acid sequence of SEQ ID NO: 10 or SEQ ID NO: 10 having at least 1 conservative substitution; and a determining region light chain 3 (CDRL3) amino acid sequence of SEQ ID NO: 11 or SEQ ID NO: 11 having at least 1 conservative substitution; and
b) a heavy chain variable region, wherein the heavy chain variable region comprises a complementarity determining region heavy chain 1 (CDRH1) amino acid sequence of SEQ ID NO: 12 or SEQ ID NO: 12 having at least 1 conservative substitution; a complementarity determining region heavy chain 2 (CDRH2) amino acid sequence of SEQ ID NO: 13 or SEQ ID NO: 13 having at least 1 conservative substitution; and a complementarity determining region heavy chain 3 (CDRH3) amino acid sequence of SEQ ID NO: 14 or SEQ ID NO: 14 having at least 1 conservative substitution.

9. The isolated antibody or antibody fragment thereof of claim 8, wherein the light chain variable region comprises the CDRL1 amino acid sequence of SEQ ID NO: 9; the CDRL2 amino acid sequence of SEQ ID NO: 10; and the CDRL3 amino acid sequence of SEQ ID NO: 11; and wherein the heavy chain variable region comprises the CDRH1 amino acid sequence of SEQ ID NO: 12; the CDRH2 amino acid sequence of SEQ ID NO: 13; and the CDRH3 amino acid sequence of SEQ ID NO: 14.

10. The isolated antibody or antibody fragment thereof of claim 8 or 9, wherein the light chain variable region amino acid comprises the sequence of SEQ ID NO: 15.

11. The isolated antibody or antibody fragment thereof of claim 8 or 9, wherein the heavy chain variable region amino acid comprises the sequence of SEQ ID NO: 16.

12. An isolated antibody or antibody fragment thereof comprising a light chain variable region amino acid sequence of SEQ ID NO: 15 and a heavy chain variable region amino acid sequence of SEQ ID NO: 16.

13. The isolated antibody or antibody fragment thereof of claim 12, wherein the light chain variable region has an amino acid sequence that is at least 90% identical to amino acid sequence of SEQ ID NO: 15.

14. The isolated antibody or antibody fragment thereof of claim 12, wherein the heavy chain variable region has an amino acid sequence that is at least 90% identical to amino acid sequence of SEQ ID NO: 16.

15. An isolated antibody or antibody fragment thereof comprising:
a) a light chain variable region, wherein the light chain variable region comprises a complementarity determining region light chain 1 (CDRL1) amino acid sequence of SEQ ID NO: 17 or SEQ ID NO: 17 having at least 1 conservative substitution; a determining region light chain 2 (CDRL2) amino acid sequence of SEQ ID NO: 2 or SEQ ID NO: 2 having at least 1 conservative substitution; and a determining region light chain 3 (CDRL3) amino acid sequence of SEQ ID NO: 18 or SEQ ID NO: 18 having at least 1 conservative substitution; and
b) a heavy chain variable region, wherein the heavy chain variable region comprises a complementarity determining region heavy chain 1 (CDRH1) amino acid sequence of SEQ ID NO: 19 or SEQ ID NO: 19 having at least 1 conservative substitution; a complementarity determining region heavy chain 2 (CDRH2) amino acid sequence of SEQ ID NO: 20 or SEQ ID NO: 20 having at least 1 conservative substitution; and a complementarity determining region heavy chain 3 (CDRH3) amino acid sequence of SEQ ID NO: 21 or SEQ ID NO: 21 having at least 1 conservative substitution.

16. The isolated antibody or antibody fragment thereof of claim 15, wherein the light chain variable region comprises the CDRL1 amino acid sequence of SEQ ID NO: 17; the CDRL2 amino acid sequence of SEQ ID NO: 2; and the CDRL3 amino acid sequence of SEQ ID NO: 18; and wherein the heavy chain variable region comprises the CDRH1 amino acid sequence of SEQ ID NO: 19; the CDRH2 amino acid sequence of SEQ ID NO: 20; and the CDRH3 amino acid sequence of SEQ ID NO: 21.

17. The isolated antibody or antibody fragment thereof of claim 15 or 16, wherein the light chain variable region amino acid comprises the sequence of SEQ ID NO: 22.

18. The isolated antibody or antibody fragment thereof of claim 15 or 16, wherein the heavy chain variable region amino acid comprises the sequence of SEQ ID NO: 23.

19. An isolated antibody or antibody fragment thereof comprising a light chain variable region amino acid sequence of SEQ ID NO: 22 and a heavy chain variable region amino acid sequence of SEQ ID NO: 23.

20. The isolated antibody or antibody fragment thereof of claim 19, wherein the light chain variable region has an amino acid sequence that is at least 90% identical to amino acid sequence of SEQ ID NO: 22.

21. The isolated antibody or antibody fragment thereof of claim 19, wherein the heavy chain variable region has an amino acid sequence that is at least 90% identical to amino acid sequence of SEQ ID NO: 23.

22. The isolated antibody or antibody fragment thereof of any of the preceding claims, wherein the antibody is recombinantly engineered, chimerized, or humanized.

23. The isolated antibody or antibody fragment thereof of any of the preceding claims, wherein the antibody is a Fab, an Fab′, an F(ab′)2, a Fv, a scFv, a diabody or fragments thereof.

24. The isolated antibody or antibody fragment thereof of any of the preceding claims, wherein the antibody binds to human streptolysin O and does not cross-react with mouse streptolysin O.

25. The isolated antibody or antibody fragment thereof of any of the preceding claims, wherein the antibody binds to streptolysin O with an affinity of greater than or equal to 40 pM.

26. The isolated antibody or antibody fragment thereof of any of the preceding claims, wherein the antibody selectively binds to human streptolysin O and inhibits binding of human streptolysin O to a cholesterol-containing host cell membrane or a carbohydrate-containing host cell membrane.

27. An isolated antibody or antibody fragment thereof comprising a light chain variable region amino acid sequence of SEQ ID NO: 7 and a heavy chain variable region amino acid sequence of SEQ ID NO: 8, wherein the isolated antibody comprises 1, 2, 3, 4, or 5 conservative amino acid substitutions in the light or heavy chain variable region amino acid sequences.

28. An isolated antibody or antibody fragment thereof comprising a light chain variable region amino acid sequence of SEQ ID NO: 14 and a heavy chain variable region amino acid sequence of SEQ ID NO: 15, wherein the isolated antibody comprises 1, 2, 3, 4, or 5 conservative amino acid substitutions in the light or heavy chain variable region amino acid sequences.

29. An isolated antibody or antibody fragment thereof comprising a light chain variable region amino acid sequence of SEQ ID NO: 22 and a heavy chain variable region amino acid sequence of SEQ ID NO: 23, wherein the isolated antibody comprises 1, 2, 3, 4, or 5 conservative amino acid substitutions in the light or heavy chain variable region amino acid sequences.

30. The isolated antibody or antibody fragment thereof of any of the preceding claims, wherein the isolated antibody or antibody fragment thereof blocks streptolysin O (SLO) binding to a host cell membrane; prevents or inhibits SLO-induced red blood cell hemolysis; or prevents or inhibits epithelial cell cytotoxicity.

31. The isolated antibody or antibody fragment thereof of any of the preceding claims, wherein the antibody is a monoclonal antibody.

32. The isolated antibody or antibody fragment thereof of any of the preceding claims, wherein the antibody is a single chain antibody.

33. The isolated antibody or antibody fragment thereof of any of the preceding claims, wherein the antibody is linked to a detectable label.

34. The isolated antibody or antibody fragment thereof of any of the preceding claims, wherein the antibody is a monovalent or a bivalent antibody.

35. The isolated antibody or antibody fragment thereof of any of the preceding claims, wherein the antibody fragment thereof is an IgG, an IgM, an IgA antibody or antigen binding fragment thereof.

36. An isolated antibody or antibody fragment thereof comprising a light chain variable region and a heavy chain variable region, wherein the light chain variable region comprises:
a) a variant complementarity determining region light chain 1 (CDRL1) comprising one or two conservative amino acid substitutions at positions 26-31 of SEQ ID NO: 7;
b) a variant complementarity determining region light chain 2 (CDRL2) comprising one or two conservative amino acid substitutions at positions 49-51 of SEQ ID NO: 7; and
c) a variant complementarity determining region light chain 3 (CDRL3) comprising one or two conservative amino acid substitutions at positions 88-98 of SEQ ID NO: 7;
wherein the heavy chain variable region comprises:
d) a variant complementarity determining region heavy chain 1 (CDRH1) comprising one or two conservative amino acid substitutions at positions 26-31 of SEQ ID NO: 8;
e) a variant complementarity determining region heavy chain 2 (CDRH2) comprising one or two conservative amino acid substitutions at positions 51-58 of SEQ ID NO: 8; and
f) a variant complementarity determining region heavy chain 3 (CDRH3) comprising one or two conservative amino acid substitutions at positions 97-117 of SEQ ID NO: 8.

37. An isolated antibody or antibody fragment thereof comprising a light chain variable region and a heavy chain variable region, wherein the light chain variable region comprises:
a) a variant complementarity determining region light chain 1 (CDRL1) comprising one or two conservative amino acid substitutions at positions 27-32 of SEQ ID NO: 15;
b) a variant complementarity determining region light chain 2 (CDRL2) comprising one or two conservative amino acid substitutions at positions 50-52 of SEQ ID NO: 15; and
c) a variant complementarity determining region light chain 3 (CDRL3) comprising one or two conservative amino acid substitutions at positions 89-98 of SEQ ID NO: 15;
wherein the heavy chain variable region comprises:
d) a variant complementarity determining region heavy chain 1 (CDRH1) comprising one or two conservative amino acid substitutions at positions 26-33 of SEQ ID NO: 16;
e) a variant complementarity determining region heavy chain 2 (CDRH2) comprising one or two conservative amino acid substitutions at positions 51-58 of SEQ ID NO: 16; and
f) a variant complementarity determining region heavy chain 3 (CDRH3) comprising one or two conservative amino acid substitutions at positions 97-119 of SEQ ID NO: 16.

38. An isolated antibody or antibody fragment thereof comprising a light chain variable region and a heavy chain variable region, wherein the light chain variable region comprises:
a) a variant complementarity determining region light chain 1 (CDRL1) comprising one or two conservative amino acid substitutions at positions 26-31 of SEQ ID NO: 22;
b) a variant complementarity determining region light chain 2 (CDRL2) comprising one or two conservative amino acid substitutions at positions 49-51 of SEQ ID NO: 22; and
c) a variant complementarity determining region light chain 3 (CDRL3) comprising one or two conservative amino acid substitutions at positions 88-98 of SEQ ID NO: 22;
wherein the heavy chain variable region comprises:
d) a variant complementarity determining region heavy chain 1 (CDRH1) comprising one or two conservative amino acid substitutions at positions 26-33 of SEQ ID NO: 23;
e) a variant complementarity determining region heavy chain 2 (CDRH2) comprising one or two conservative amino acid substitutions at positions 51-58 of SEQ ID NO: 23; and
f) a variant complementarity determining region heavy chain 3 (CDRH3) comprising one or two conservative amino acid substitutions at positions 97-119 of SEQ ID NO: 23.

39. A composition comprising an isolated antibody or antibody fragment thereof of any of the preceding claims, and at least one pharmaceutically acceptable carrier or diluent.

40. The composition of claim 39, further comprising a detectable label or reporter.

41. A method of treating a necrotizing soft tissue infection in a subject, the method comprising administering to the subject a therapeutically effective amount of an isolated antibody or antibody fragment thereof of any of claims 1-38 or the composition of any of claims 39-40.

42. A method of inhibiting or preventing streptolysin O-induced RBC-hemolysis in a subject, the method comprising administering to the subject a therapeutically effective amount of an isolated antibody or antibody fragment thereof of any of claims 1-38 or the composition of any of claims 39-40.

43. A method of preventing or inhibiting cytotoxicity in epithelial cells in a subject, the method comprising administering to the subject a therapeutically effective amount of an isolated antibody or antibody fragment thereof of any of claims 1-38 or the composition of any of claims 39-40.

44. The method of claim 42 or 43, wherein the subject has a necrotizing soft tissue infection.

45. The method of claim 41 or 44, wherein the necrotizing soft tissue infection is fasciitis, myositis or cellulitis.

46. The method of any of claims 41-45, wherein the subject is identified in need of treatment before the administering step.

47. The method of any of claims 41-46, wherein the antibody or antibody fragment thereof is administered in a pharmaceutically acceptable composition.

48. The method of any of claims 41-47, wherein the antibody or antibody fragment thereof is administered systemically, intravenously, intradermally, intramuscularly, intraperitoneally, subcutaneously or locally into inflamed tissues or organs.

49. The method of any of claims 41-48, further comprising administering one or more of imipenem, meropenem, ertapenem, piperacillin (tazobactam), tigecycline, penicillin, clindamycin, a fluoroquinolone or an aminoglycoside, vancomycin, daptomycin, linezolid, azithromycin, or erythromycin to the subject.

50. The method of any of claims 41-49, wherein the subject is a human.

51. The method of any of claims 41-50, wherein the antibody is a monoclonal antibody.

52. A vector comprising a sequence of encoding the isolated antibody or antibody fragment thereof of any of claims 1-38.
